(19) United States
(12) Patent Application Publication
(10) Pub. No.: US 2025/0099634 A1
(43) Pub. Date: Mar. 27, 2025

(54) METHOD OF TREATING CANCER

(71) Applicant: Novartis AG, Basel (CH)

(72) Inventors: Per Ernst-Uno Broberg, Limhamn (SE); Juergen Herbert Fleck, Bahlingen (DE); Ilia Folitar, Riehen (CH); Ivana Galetic, Allschwil (CH); Germo Hans Gericke, Fischingen (DE); Euloge Kpamegan, East Hanover, NJ (US); Raya Toteva Kozhuharova, Geneva (CH); Maurizio F. Mariani, Ivrea (IT); Kalidas Menon Elangunthazatx, Oullins (FR); Paola Maria Santoro, Reading, MA (US); Amrita Gill Sawhney, East Hanover, NJ (US); Antje Wegener, Saint-Nom-La-Bretèche (FR)

(21) Appl. No.: 18/819,282

(22) Filed: Aug. 29, 2024

Related U.S. Application Data
(60) Provisional application No. 63/584,928, filed on Sep. 25, 2023.
(60) Provisional application No. 63/621,705, filed on Jan. 17, 2024.

(51) Int. Cl. A61K 51/08

(52) U.S. Cl. CPC A61K 51/083; A61K 2121/00

(57) ABSTRACT

The present disclosure relates to a method of treating well-differentiated G2 or G3 neuroendocrine tumor (NET) in a patients in need thereof, comprising administering to said patient a treatment comprising a therapeutically effective dose of a somatostatin receptor (SSTR) targeting radioligand therapeutic (RLT) agent, in particular [¹⁷⁷Lu]Lu-DOTA⁰-Tyr³-octreotate, as first line therapy.

### FIELD OF THE INVENTION

The present disclosure relates to methods of treating well-differentiated G2 or G3 neuroendocrine tumor (NET) in a patients in need thereof, comprising administering to said patient a treatment comprising a therapeutically effective dose of a somatostatin receptor (SSTR) targeting radioligand therapeutic (RLT) agent, as first line therapy.

### BACKGROUND OF THE INVENTION

Neuroendocrine tumors (NET) are rare malignant neoplasms that can arise throughout the body, and account for approximately 1% of all human tumors. NETs have been classified according to their embryonic origin as foregut, midgut, or hindgut NETs. The World Health Organization (WHO) staging system classifies gastroenteropancreatic NET (GEP-NET) based on primary tumor localization, size, mitotic activity, invasiveness, and functional status (Klöppel, 2011). Despite certain common morphological and immunohistochemical features, there is significant heterogeneity in the prognosis and treatment strategies according to the primary site, histological differentiation (poorly or well-differentiated) and stage. Histologic differentiation and proliferative activity are the strongest predictors of survival. In terms of site of origin, the majority (50-70%) of NETs diagnosed in Western countries are gastrointestinal NETs (Modlin, Lye, & Kidd, 2003). Of 270 NETs originating in the midgut or hindgut, 62% (5-year survival rate (YSR), 95.2%) were Grade 1, 32% (5-YSR 82.0%) were Grade 2, and 6% (5-YSR, 51.4%) were Grade 3 NETs (Jann, et al., 2011). 50-70% of NETs of unknown origin have been reported to be well-differentiated (Catena, et al., 2011) and have a similar behavior and prognosis to midgut NETs (Kirshbom, Kherani, Onaitis, Feldman, & Tyler, 1998).

Surgical resection of the primary and metastatic lesions remains the mainstay of treatment, and the only way to obtain a cure. However, resection is often not possible as NETs are frequently detected in a more advanced tumor stage (Yao, et al., 2008).

In patients with inoperable NET, the treatment goal is to prolong survival, improve and maintain quality of life, to control tumor growth, and to control secretory symptoms (if the tumor is functional). Somatostatin analogs (SSAs) octreotide long-acting 30 mg (Sandostatin® LAR®) or lanreotide ATG 120 mg (lanreotide ATG, SOMATULINE® DEPOT or SOMATULINE® AUTOGEL®) have become the mainstay of treatment in patients with low- or intermediate-grade GEP-NET.

In the PROMID study, 42 patients with metastatic midgut NET who received octreotide long-acting 30 mg/month had more than double the time to tumor progression compared with 43 patients who received placebo (14.3 versus 6.0 months, respectively; P=0.000072). Overall, 67% of patients treated with octreotide long-acting achieved stable disease compared with 37% of patients who received placebo (Rinke, et al., 2009). In the CLARINET study, patients with G1-2 midgut and pancreatic NET treated with lanreotide 120 mg/month had significant prolongation of progression free survival (PFS) when compared to placebo (median PFS not reached vs. 18.0 months, P<0.001; HR for progression or death with lanreotide vs. placebo, 0.47; 95% CI 0.30 to 0.73) (Caplin, et al., 2014).

Where approved, the dose for octreotide long-acting for the treatment of advanced midgut NET is 30 mg/month. Higher doses of octreotide up to 120 mg per month have been used for symptom control in patients no longer responding adequately to standard doses. Higher doses have also been used for tumor control though controlled studies are lacking (Broder, Beenhouwer, Strosberg, Neary, & Cherepanov, 2015). Three studies have investigated the antiproliferative efficacy of high-dose treatment with either octreotide long-acting (up to 160 mg every 2 weeks) or lanreotide (up to 15 mg/d), showing disease stabilization in 37-75% of advanced midgut carcinoid and/or metastatic GEP-NET patients (Eriksson, Renstrup, Imam, & Oberg, 1997; Faiss, et al., 1999; Welin, et al., 2004).

Furthermore, retrospective analyses suggest that higher doses of octreotide may delay the time to tumor progression/other types of intervention (Lau, Abdel-Rahman, & Cheung, 2018; Chadha, et al., 2009). In a small (n=28) sequential study, increased frequency of dosing of somatostatin analogs resulted in delayed time to tumor progression and time to biochemical progression (Ferolla, et al., 2012).

In patients whose disease has progressed, mTOR therapy (everolimus), tyrosine kinase inhibitors (sunitinib) and peptide radionuclide receptor therapy (PRRT) provide treatment options (Yao, et al., 2010; Yao, et al., 2011; Pavel, et al., 2011; Yao, et al., 2016; Kwekkeboom & Krenning, Peptide Receptor Radionuclide Therapy in the Treatment of Neuroendocrine Tumors., 2016). Currently there is no broadly accepted standard chemotherapy for the treatment of NET. A combination of streptozotocin and 5-flourouracil or doxorubicin is frequently used in patients with well-differentiated pancreatic NET (PNET), with inoperable progressive liver metastasis, but there is no robust evidence to support the use of chemotherapy in patients with NETs of other origin (Pavel, et al., 2012). Within the group of neuroendocrine neoplasm (NEN) G3, the distinction between NET G3 and neuroendocrine carcinomas (NEC) G3 is clinically meaningful. NET G3 and NEC are characterized by significant differences in Ki67 index (tumors with a Ki67 index between 20 and 55% are less aggressive than tumors with a Ki67 index above 55%). While platinum-based chemotherapy is effective in NEC, it seems to have limited value in NET G3. Treatments established for NET G2 such as temozolomide based chemotherapy or PRRT may be considered for the treatment of NET G3 (Rinke 2017).

Tumor-targeted peptide receptor radionuclide therapy (PRRT) has been under clinical evaluation since 1992 for tumors expressing somatostatin receptors. The biological basis for radionuclide receptor imaging and receptor targeted radionuclide therapy is the receptor-mediated internalization and intracellular retention of radiolabelled somatostatin analogues. Sst₂ receptors are an attractive target for PRRT because the receptor density is higher on tumor than on non-tumor tissue (Reubi, Waser, Schaer, & Laissue, 2001; Reubi J. C., 2003), and because sst₂ receptors internalize into cells after ligand binding. Consequently, the radioactivity delivered by the radiolabelled peptide is captured in the target cell after binding to the sst₂ receptor (Reubi, et al., 2000).

¹⁷⁷Lu-DOTA⁰-Tyr³-Octreotate (¹⁷⁷Lu-Dotatate, Lutathera®) consists of a somatostatin peptide analogue, coupled to the metal-ion chelating moiety, DOTA, and radiolabelled with ¹⁷⁷Lu. ¹⁷⁷Lu-DOTA⁰-Tyr³-Octreotate binds with high-affinity to somatostatin receptors and retains its binding properties and physiological functions when complexed with ¹⁷⁷Lu. ¹⁷⁷Lu emits low to intermediate-energy beta-particles with an Eₘₐₓ of 0.5 MeV, and which have a tissue penetration range of up to 2 mm. The relatively short penetration range of ¹⁷⁷Lu betas leaves more of the radiation dose in the tumor with less loss to the surrounding tissues. There is rapid urinary clearance of radiolabelled ¹⁷⁷Lu-DOTA⁰-Tyr³-Octreotate from the circulation, which gives this radiopharmaceutical a major advantage over other approaches, such as cell targeted radiolabelled antibodies.

Because of ¹⁷⁷Lu-DOTA⁰-Tyr³-Octreotate mechanism of action, there is some radioactivity retention in the kidneys, however concomitant administration of the amino acids lysine and arginine reduces renal uptake of radioactivity without altering tumor uptake (Kwekkeboom, et al., 2001; Strosberg, et al., 2017). This phenomenon of protection mediated by co-infusion of a lysine and arginine solution occurs by “blocking” the mechanism for renal tubular uptake of proteins or peptides (Hammond, et al., 1993; Rolleman, Valkema, Jong, Kooij, & Krenning, 2003). The co-administration of 2.5% lysine and arginine (Lys-Arg) amino acid solution yields about 33% inhibition of renal uptake of radioactivity at 24 hours and is better tolerable compared to the commercial amino acid solutions, specifically in terms of nausea and vomiting, due to the higher osmolality of the more complex formulated commercial AA solutions (Rolleman, Valkema, Jong, Kooij, & Krenning, 2003; Kwekkeboom, et al., 2008). Common side effects of Lutathera include lymphopenia, increased GGT, AST and/or ALT, vomiting, nausea, hyperglycemia and hypokalemia. Serious side effects of Lutathera include myelosuppression, secondary myelodysplastic syndrome and leukemia, renal toxicity, hepatotoxicity, neuroendocrine hormonal crises and infertility.

Lutathera (lutetium (¹⁷⁷Lu) oxodotreotide) was approved in Europe on Sep. 26, 2017 for the treatment of unresectable or metastatic, progressive, well differentiated (G1 and G2) GEP-NETs and in the USA on 26 Jan. 2018 for the treatment of somatostatin receptor positive GEP-NETs.

The approval of Lutathera was supported by two studies:
The first was the Phase III NETTER-1 study sponsored by Advanced Accelerator Applications. This was multicenter, stratified, open, randomized, comparator-controlled, parallel-group study, comparing treatment with ¹⁷⁷Lu-DOTA⁰-Tyr³-Octreotate (Lutathera, lutetium Lu 177 dotatate) plus best supportive care (30 mg octreotide long-acting) to treatment with high dose (60 mg) octreotide long-acting in patients with inoperable, somatostatin receptor positive, histologically proven midgut carcinoid tumors, progressive under octreotide long-acting (Strosberg, et al., 2017; Strosberg, et al., 2018). A significant improvement in PFS, the primary endpoint, was demonstrated. There were 21 confirmed events (disease progressions according to RECIST 1.1 centrally assessed or deaths without confirmed progression) in the Lutathera arm and 70 events in the octreotide long-acting 60 mg arm. The median PFS for the control arm was 8.5 months while the median PFS for the Lutathera arm had not yet been reached. The improvement in median PFS in the Lutathera arm was statistically significant with a hazard ratio of 0.18 (95% CI, 0.11-0.29), demonstrating a risk reduction of 79% in the likelihood of having a progression or death event when treated with Lutathera versus 60 mg octreotide long-acting.
The second study was based on data from 1,214 patients with somatostatin receptor-positive tumors, including GEP-NETS, who received Lutathera at the Erasmus Medical Centre in the Netherlands (Kwekkeboom, et al., 2008; Brabander, et al., Long-Term Efficacy, Survival, and Safety of [177Lu-DOTA0, Tyr3] octreotate in Patients with Gastroenteropancreatic and Bronchial Neuroendocrine Tumors., 2017a). Complete or partial tumor remission was reported in 39% of patients.

On Jun. 2, 2016 and 15 Dec. 2016 Advanced Accelerator Applications obtained marketing approval from the FDA for NETSPOT™ (68Ga-Dotatate) and from the European Commission for Somakit-TOC™ (68Ga-Dotatoc), the diagnostic companions of Lutathera. Neuroendocrine tumors (NET) are rarely seen in childhood. The incidence in children and adolescents is low at 2.8 per million persons under the age of 30 years. Despite their low incidence, NETs represent the most frequent tumor of the gastrointestinal tract in children (Howell & O'Dorisio, 2012). Currently all treatments approved for GEP-NETs are for adults only.

### SUMMARY OF THE INVENTION

The pivotal Phase III NETTER-1 study showed that Lutathera with best supportive care (30 mg octreotide long-acting) provided a significant increase in PFS to patients with progressive midgut carcinoid tumors (at enrollment) compared to patients treated with high dose (60 mg) octreotide long-acting. The NETTER-1 patient population included 34.5% of patients with G2 NET (65.5% G1), while G3 NETs were excluded. Only patients progressive on SSAs were eligible (2^{nd} line), SSA-naïve patients were excluded.

The aim of the NETTER-2 study, that relates to the present invention, is to determine if Lutathera in combination with octreotide long-acting prolongs PFS in GEP-NET patients with high proliferation rate tumors (G2 and G3), when given as a 1^{st} line treatment in comparison to treatment with high dose (60 mg) octreotide long-acting. SSA-naïve patients are eligible, as well as patients previously treated with SSAs in the absence of progression. Based on extensive experience with Lutathera as well as octreotide LAR in adult GEP-NET patients, and the relevance of the molecular target in adolescent GEP-NET patients, the study will be open to adolescents aged ≥15 years and >40 kg body weight (BW); younger patients are not expected to present with the disease meeting the severity criteria for this trial. Due to the rarity of the disease, and specifically GEP-NETs with the severity assessed in this study, no minimum number of adolescent patients is required. The study has been open to adolescents to ensure that access is not unnecessarily denied.

In addition, in the NETTER-2 study, any progressive patient in the high dose octreotide long acting arm has the option, if eligible, to enroll for post-progression cross-over with Lutathera.

Furthermore, patients who experience disease progression in the Lutathera arm after having received and benefitted from 4 doses/cycles in an initial treatment have the option, if eligible according to criteria described below, to enroll for re-treatment phase with Lutathera.

The results from the NETTER-2 study, as disclosed herein, indicate the efficacy and safety of Lutathera in patients ≥15 years of age with Grade 2 and 3 advanced GEP-NET (Ki67 index of ≥10 and ≤55%), considered being a candidate for treatment with high dose octreotide long-acting.

The radioligand therapy Lutathera® demonstrated statistically significant and clinically meaningful progression-free survival in first line advanced gastroenteropancreatic neuroendocrine tumors (GEP-NETs).

Phase III NETTER-2 trial met primary endpoint of improvement in progression-free survival (PFS) and key secondary endpoint of objective response rate (ORR) in patients with Grade 2 and 3 advanced gastroenteropancreatic neuroendocrine tumors (GEP-NETs) who received first line treatment with Lutathera® in combination with long-acting octreotide, versus high-dose long-acting octreotide alone [1,2]

Lutathera is the first radioligand therapy (RLT) to demonstrate clinically meaningful benefit in a first line setting [1].

The Phase III NETTER-2 trial with Lutathera® (INN: lutetium (177Lu) oxodotreotide/USAN: lutetium Lu 177 dotatate) met its primary endpoint. First line treatment with Lutathera in combination with long-acting octreotide demonstrated a significant improvement in progression-free survival (PFS) in patients with newly diagnosed somatostatin receptor (SSTR)-positive, Grade 2 and 3, advanced gastroenteropancreatic neuroendocrine tumors (GEP-NETs) versus high-dose long-acting octreotide alone [1,2]. No new or unexpected safety findings were observed in the study and data are consistent with the already well-established safety profile of Lutathera [1-4].

NETs are a type of cancer that originate in neuroendocrine cells throughout the body and are commonly considered slow-growing malignancies. However, some NETs are associated with rapid progression and poor prognosis and in many cases, diagnosis is delayed until patients have advanced disease [5-7]. Even though NETs are a rare (orphan) disease, their incidence has grown over 500% in the last three decades [5-8] and there is an urgent need for additional treatment options for patients newly diagnosed with inoperable or advanced disease.

With these results, NETTER-2 is Lutathera's second Phase III trial showing clinically meaningful results for patients [2,4]. The approval of Lutathera was originally based on the pivotal NETTER-1 trial, which demonstrated highly significant and clinically meaningful PFS prolongation for patients treated with Lutathera in combination with long-acting octreotide versus high-dose (60 mg) long-acting octreotide for SSTR-positive, inoperable midgut neuroendocrine tumors (NETs) who were progressing despite standard treatment [3-4,9].

These positive results for Lutathera are quite remarkable and they represent the potential for radioligand therapy to make a meaningful impact for newly diagnosed patients living with advanced GEP-NETs. The use of radioligand therapies in earlier lines of treatment for patients with cancer precisely deliver novel treatment modalities directly to the cancer cells to improve patient outcomes.

NETTER-2 (NCT03972488) is an open-label, multi-center, randomized, comparator-controlled Phase III trial assessing whether Lutathera plus long-acting octreotide when taken as a first line treatment can prolong PFS in patients with high-proliferation rate tumors (G2 and G3), compared to treatment with high-dose (60 mg) long-acting octreotide². Eligible patients were diagnosed with SSTR-positive advanced GEP-NETs within 6 months before enrollment [2].

Lutathera® (INN: lutetium (¹⁷⁷Lu) oxodotreotide/USAN: lutetium Lu 177 dotatate) is an Advanced Accelerator Applications RLT approved in the United States for the treatment of SSTR-positive GEP-NETs, including foregut, midgut and hindgut neuroendocrine tumors in adults and in Europe for unresectable or metastatic, progressive, well-differentiated (G1 and G2), SSTR-positive GEP-NETs in adults [10-11].

The present disclosure is about cancer care with RLT for patients with advanced cancers. By harnessing the power of radioactive atoms and applying it to advanced cancers, RLT is theoretically able to deliver radiation to target cells anywhere in the body [12-13].

### REFERENCES (FOR THE PRECEDING PARAGRAPHS)

1. See data as disclosed herein.
2. ClinicalTrials.gov. NETTER-2 (NCT03972488). Study to Evaluate the Efficacy and Safety of Lutathera in Patients With Grade 2 and Grade 3 Advanced GEP-NET (NETTER-2). Accessed Sep. 15, 2023. https://classic.clinicaltrials.gov/ct2/show/NCT03972488
3. Strosberg J R, Caplin M E, Kunz P L, et al. ¹⁷⁷Lu-Dotatate plus long-acting octreotide versus high-dose long-acting octreotide in patients with midgut neuroendocrine tumours (NETTER-1): final overall survival and long-term safety results from an open-label, randomised, controlled, phase 3 trial [published correction appears in Lancet Oncol. 2022 February; 23(2):e59]. Lancet Oncol. 2021; 22 (12): 1752-1763. doi: 10.1016/S1470-2045 (21) 00572-6
4. NETTER-1 Phase III in Patients With Midgut Neuroendocrine Tumors Treated With ¹⁷⁷Lu-DOTATATE: Efficacy and Safety Results. Clin Adv Hematol Oncol. 2016; 14 (5 Suppl 7): 8-9.
5. Man D, Wu J, Shen Z, Zhu X. Prognosis of patients with neuroendocrine tumor: a SEER database analysis. Cancer Manag Res. 2018; 10:5629-5638. Published 2018 Nov. 13. doi: 10.2147/CMAR.S174907Dasari
6. A, Shen C, Halperin D, Zhao B, Zhou S, Xu Y, Shih T, Yao J C. Trends in the Incidence, Prevalence, and Survival Outcomes in Patients With Neuroendocrine Tumors in the United States. JAMA Oncol. 2017; doi: 10.1001/jamaoncol.2017.0589
7. Frilling A, Åkerström G, Falconi, M, et al. Neuroendocrine tumor disease: an evolving landscape. Endoc Related Cancer 2012; 19: R163-815.
8. Lawrence B, Gustafsson B I, et al. The Epidemiology of Gastroenteropancreatic Neuroendocrine Tumors. Endocrinol Metab Clin N Am. 2011; 40:1-18
9. Novartis. Press Release. Available online: https://www.novartis.com/news/media-releases/advanced-accelerator-applications-receives-fda-approval-lutathera-treatment-gastroenteropancreatic-neuroendocrine-tumors. Last accessed September 2023.
10. Lutathera. Full Prescribing 2018. Information. Revised March 2023. https://www.novartis.com/us-en/sites/novartis_us/files/lutathera.pdf
11. Lutathera. Summary of Product Characteristics (SmPC). 2018. Revised February 2023. https://www.ema.europa.eu/en/documents/product-information/lutathera-epar-product-information_en.pdf
12. Jadvar H. Targeted radionuclide therapy: an evolution toward precision cancer treatment. AJR Am J Roentagenol. 2017; 209 (2); 277-288.
13. Jurcic J G, Wong J Y C, Knoc S J, et al. Targeted radionuclide therapy. In: Tepper J E, Foote R E, Michalski J M, eds. Gunderson & Tepper's Clinical Radiation Oncology. 5th ed. Elsevier, Inc. 2021; 71 (3): 209-249

The Phase III NETTER-2 trial with Lutathera® met its primary endpoint of improvement in progression-free survival (PFS) and a key secondary endpoint of objective response rate (ORR) in patients with well-differentiated Grade 2 and 3 advanced gastroenteropancreatic neuroendocrine tumors (GEP-NETs) [1,2].

With the data disclosed herein, Lutathera is the first radioligand therapy (RLT) to demonstrate statistically significant and clinically meaningful benefit in a first line setting [1].

The Phase III NETTER-2 trial is assessing whether Lutathera plus long-acting octreotide when taken as a first line treatment can prolong progression-free survival (PFS) in patients with Grade 2 and 3 (defined as: mitotic count>2 and Ki67>3%) high-proliferation rate gastroenteropancreatic neuroendocrine tumors (GEP-NETs), compared to treatment with high-dose (60 mg) long-acting octreotide alone, whereas the Phase III NETTER-1 trial explored the same treatment protocol in patients with midgut NET who had already taken standard treatment and were still progressing [2,6,7]. NETTER-2 is also investigating patients with a more aggressive form of disease than was included in the patient population for NETTER-1.

NETTER-2 is the first clinical trial to show statistically significant benefits of a radioligand therapy (RLT) in a first line setting [1]. The results collected so far offer evidence of the benefit of RLTs as a first line treatment.

NETTER-2 met its primary endpoint of progression-free survival (PFS) and demonstrated statistical significance in objective response rate (ORR), a key secondary endpoint [1].

No new or unexpected safety findings were observed in NETTER-2 and data are consistent with the already well-established safety profile of Lutathera [1,8,9].

The Phase III NETTER-2 trial also provides the first incidence of a safety profile for Lutathera with 2.5% lysine-arginine solution [1,2]. NETTER-1, in contrast, used complex amino acid solutions, which contribute to a higher frequency of adverse reactions (e.g. nausea and vomiting), attributed to the emetic effect of the concomitant amino acids infusion [1,8,9].

### REFERENCES (FOR THE PRECEDING PARAGRAPHS)

1. Data as disclosed herein.
2. ClinicalTrials.gov. NETTER-2 (NCT03972488). Study to Evaluate the Efficacy and Safety of Lutathera in Patients With Grade 2 and Grade 3 Advanced GEP-NET (NETTER-2). Accessed Sep. 15, 2023. https://clinicaltrials.gov/ct2/show/NCT03972488
3. Lutathera. Full Prescribing Information. 2018. Revised March 2023. https://www.novartis.com/us-en/sites/novartis_us/files/lutathera.pdf
4. Lutathera. Summary of Product Characteristics (SmPC). 2018. Revised February 2023. https://www.ema.europa.eu/en/documents/product-information/lutathera-epar-product-information_en.pdf
5. Lutathera. Product monograph. 2019. Revised May 2021. https://ed2b79.p3cdn1.secureserver.net/wp-content/uploads/2022/02/Lutathera-PM-E.pdf
6. ClinicalTrials.gov. NETTER-1 (NCT01578239). A Study Comparing Treatment With 177Lu-DOTA0-Tyr3-Octreotate to Octreotide LAR in Patients With Inoperable, Progressive, Somatostatin Receptor Positive Midgut Carcinoid Tumours. Accessed Sep. 15, 2023. https://clinicaltrials.gov/study/NCT01578239
7. American Society of Clinical Oncology (ASCO). Cancer.net. Neuroendocrine Tumors: Grades. May 2022. Accessed Sep. 22, 2023. https://www.cancer.net/cancer-types/neuroendocrine-tumors/grades
8. Strosberg J R, Caplin M E, Kunz P L, et al. ¹⁷⁷Lu-Dotatate plus long-acting octreotide versus high-dose long-acting octreotide in patients with midgut neuroendocrine tumours (NETTER-1): final overall survival and long-term safety results from an open-label, randomised, controlled, phase 3 trial [published correction appears in Lancet Oncol. 2022 February; 23 (2): e59]. Lancet Oncol. 2021; 22 (12): 1752-1763. doi: 10.1016/S1470-2045 (21) 00572-6
9. NETTER-1 Phase III in Patients With Midgut Neuroendocrine Tumors Treated With 177Lu-DOTATATE: Efficacy and Safety Results. Clin Adv Hematol Oncol. 2016; 14 (5 Suppl 7): 8-9.
10. Novartis. Press Release. Available online: https://www.novartis.com/news/media-releases/advanced-accelerator-applications-receives-fda-approval-lutathera-treatment-gastroenteropancreatic-neuroendocrine-tumors. Last accessed September 2023.

### The Present Disclosure Provides:

A method of treating well-differentiated G2 or G3 neuroendocrine tumor (NET) in a patients in need thereof, comprising administering to said patient a treatment comprising a therapeutically effective dose of a somatostatin receptor (SSTR) targeting radioligand therapeutic (RLT) agent as first line therapy.

A method of delaying the time-to-first-occurrence progression or death in a patient with well-differentiated G2 or G3 neuroendocrine tumor (NET), comprising administering to said patient a treatment comprising a therapeutically effective dose of a somatostatin receptor (SSTR) targeting radioligand therapeutic (RLT) agent as first line therapy.

A method of delaying the need for the initiation of chemotherapy in a patient with well-differentiated G2 or G3 neuroendocrine tumor (NET), comprising administering to said patient a treatment comprising a therapeutically effective dose of a somatostatin receptor (SSTR) targeting radioligand therapeutic (RLT) agent as first line therapy.

A method of controlling the disease in a patient with well-differentiated G2 or G3 neuroendocrine tumor (NET), comprising administering to said patient a treatment comprising a therapeutically effective dose of a somatostatin receptor (SSTR) targeting radioligand therapeutic (RLT) agent as first line therapy.

A method of remitting well-differentiated G2 or G3 neuroendocrine tumor (NET) in a patient in need thereof, comprising administering to said patient a treatment comprising a therapeutically effective dose of a somatostatin receptor (SSTR) targeting radioligand therapeutic (RLT) agent as first line (1L) therapy.

A method of reducing the risk of progression of well-differentiated G2 or G3 neuroendocrine tumor (NET) in a patient in need thereof or reducing the risk of death of said patient, comprising administering to said patient a treatment comprising a therapeutically effective dose of a somatostatin receptor (SSTR) targeting radioligand therapeutic (RLT) agent as first line (1L) therapy.

### BRIEF DESCRIPTION OF THE DRAWINGS

FIG. 1 shows the design of the clinical trial.

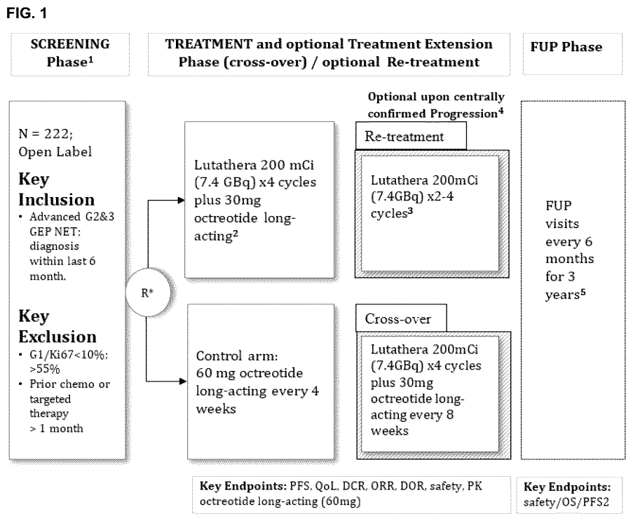

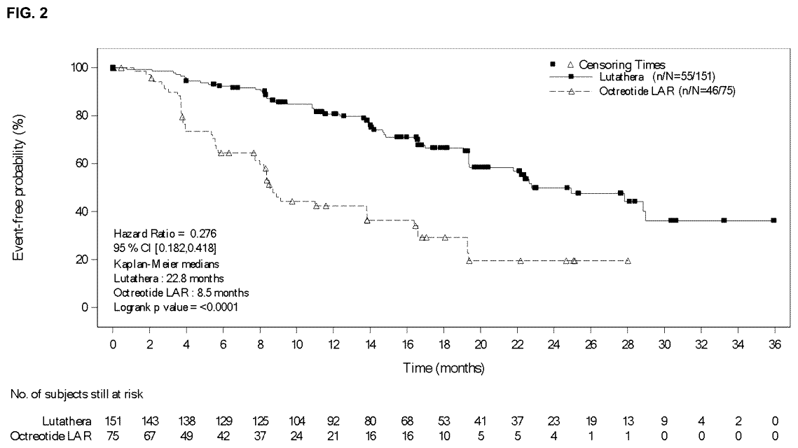

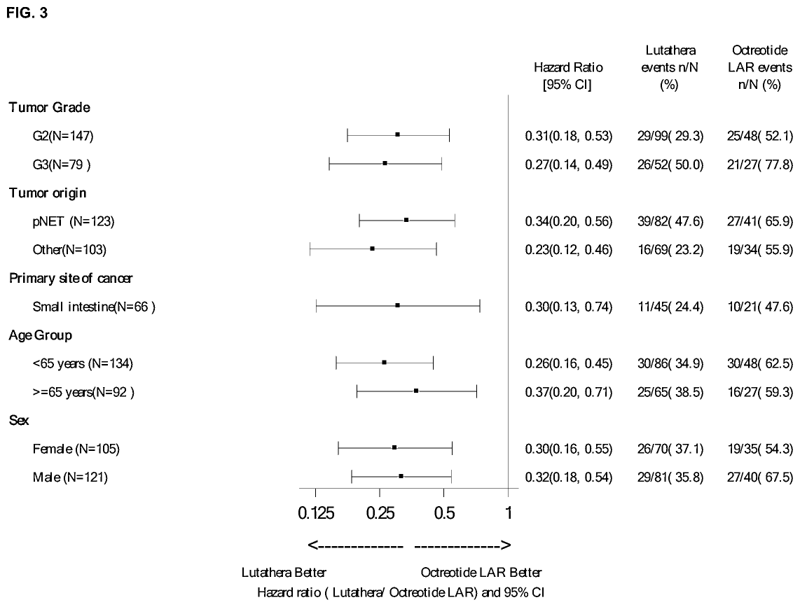

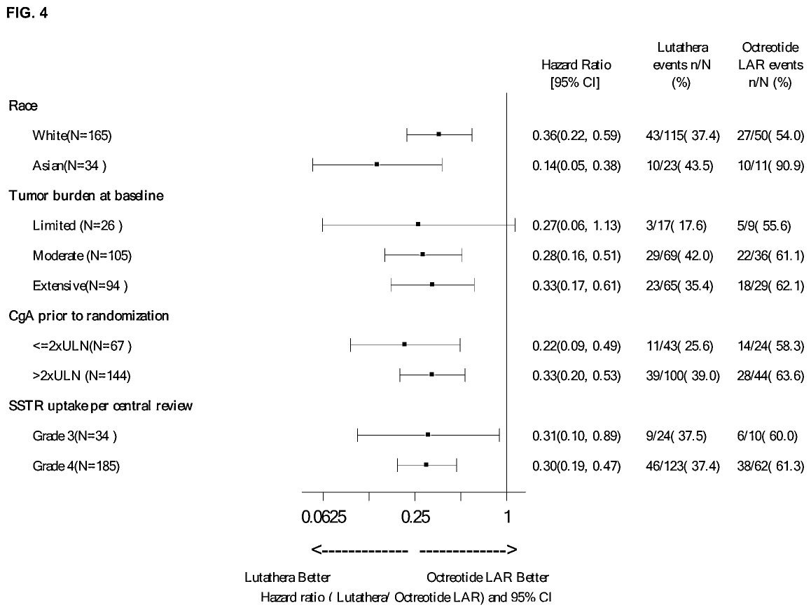

The footnotes relating to this FIG. 1 are provided in the following:
(1) Randomization ratio: 2:1. Stratification Factors: Grade (G2 vs G3) and Tumour Origin (pNET vs other origin).
(2) Octreotide long-acting 30 mg every 8 weeks during Lutathera treatment and every 4 weeks after last Lutathera treatment.
(3) Somatostatin analogues in re-treatment phase are at discretion of investigator
(4) If RECIST progression occurs after Week 72 post the primary end point analysis, the decision to enroll the patient in the cross-over or re-treatment phase will be based on local assessment.
(5) Patients included in the optional cross-over or re-treatment phase will be followed up for 3 years or until EoS, whichever occurs first.

FIG. 2 shows the Kaplan-Meier plot of Progression-Free Survival (PFS) (months) based on central review and using RECIST 1.1 criteria (Full Analysis Set).

FIGS. 3 and 4 show the Forest plots of Progression-Free Survival (PFS) (months) based on central review and using RECIST 1.1 criteria-Key subgroups of interest (Full Analysis Set).

### DETAILED DESCRIPTION OF THE INVENTION

Herein after, the present disclosure is described in further detail and is exemplified.

### Study Design

This is a multicenter, stratified, randomized, open-label comparator-controlled, Phase III study in patients ≥15 years with somatostatin receptor positive, well-differentiated G2 and G3, advanced GEP NETs, diagnosed within 6 months prior to screening.

Overall, 222 patients will be randomized (2:1 randomization ratio) to receive treatment with Lutathera (7.4GBq/200 mCi×4 administrations every 8±1 weeks; cumulative dose: 29.6 GBq/800mCi) plus octreotide long-acting (30 mg every 8 weeks during Lutathera treatment and every 4 weeks after last Lutathera treatment) or high dose octreotide long-acting (60 mg every 4 weeks). Randomization will be stratified by Grade (G2 vs G3) and tumor origin (pNET vs other origin) (FIG. 1).

The primary endpoint of the study is PFS centrally assessed (target HR=0.5; 90% power, 1-sided α=2.5%). The primary analysis will be performed after 99 PFS events (99 evaluable and centrally confirmed disease progressions or death events) have occurred.

The study consists of a Screening Phase, a Treatment Phase, an optional Treatment Extension Phase (cross-over)/an optional Re-treatment Phase and a Follow up Phase.

### Screening Phase

The screening phase should be shortened as much as possible, in order to treat the patients possibly within 2 weeks after the consent signature.

The randomization should be performed as soon as possible, after all eligibility criteria are verified. Patients under octreotide long-acting before study entry should have their last dose carefully programmed during the screening phase (in case of randomization in the Lutathera arm a washout period of 6 weeks from octreotide long-acting must be observed prior to Lutathera injection).

As Lutathera production and shipment will take circa 12 days to be arranged, if a patient is randomized in the Lutathera arm, Lutathera first dose should be ordered within 24 h after randomization.

The baseline CT/MRI scan should be taken possibly on the same day as randomization or immediately before (within 1 week) to ensure that it reflects the disease status closely before the therapy start.

### Treatment Phase

During the Treatment Phase, patients will be followed according to the assessments schedule Objective tumor response will be assessed at W16±1, W24±1 and then every 12±1 weeks from the randomization date, according to RECIST 1.1 criteria (central+local assessment up to first progression, then only local assessment).

### Duration of the Treatment Phase:

Before the PFS primary analysis:
The Treatment Phase is not fixed and the patients who are randomized should continue to receive the study treatments until progression or death (patients randomized in the Lutathera arm, will continue octreotide long-acting 30 mg injections after the completion of the 4 Lutathera treatments until progression or death; patients randomized in the octreotide long-acting arm will continue octreotide long-acting 60 mg injections until progression or death).
After the PFS primary analysis:
The Treatment Phase becomes fixed and will stop at week 72 (patients randomized in the Lutathera arm, will continue octreotide long-acting 30 mg injections after the completion of the 4 Lutathera treatments until week 72 or earlier in case of progression or death; patients randomized in the octreotide long-acting arm will continue octreotide long-acting 60 mg injections until week 72 or earlier in case of progression or death).

In other words, before the PFS primary analysis (i.e. 99 evaluable and centrally confirmed disease progressions or death events), patients continue the Treatment Phase until progression; after the PFS primary analysis, the Treatment Phase duration is limited to 72 weeks.

At any time during the study (before or after the PFS primary analysis) any progressive patient (based on central imaging assessment) immediately ceases the Treatment Phase and proceeds to the Follow-up Phase. If a patient discontinues Lutathera before centrally confirmed disease progression, the patient should be followed with tumor assessments as per initial CT/MRI schedule until centrally confirmed disease progression.

In addition, patients randomized in the control arm have the option, if eligible, to enroll for post-progression cross-over with Lutathera. Control arm patients who cross-over after progression will be allowed to complete the Lutathera treatments after week 72 or to cross-over during the Follow-up Phase, in case of late progression. Furthermore, patients who experience disease progression in the Lutathera arm have the option, if eligible, to enroll for re-treatment phase with Lutathera.

Patients included in the optional crossover or re-treatment phase will be followed up at least 6 months and up to 3 years (or until EoS, whatever comes first).

### Optional Treatment Extension Phase (Cross-Over)

In the control arm, any RECIST progressive patient (based on central assessment) has the option to enroll for post-progression cross-over, upon signature of a new consent, to receive maximum 4 cycles of Lutathera (7.4 GBq/200 mCi x 4 cycles; cumulative dose: 29.6 GBq/800mCi) plus 30 mg octreotide long acting every 8 weeks.

If RECIST progression occurs after Week 72 post the primary end point analysis, the decision to enroll the patient in cross-over will be based on local assessment. The cross-over phase will last until the completion of four Lutathera administrations after which patients will undergo EOT visit (at Week 28), and proceed to the follow up phase. After cross-over phase is complete, patients will be followed up for 3 years or until EoS, whichever occurs first (and at least 6 months after the last Lutathera dose administered during cross-over).

The time window to start cross-over treatment with Lutathera in this study is 4 years after last patient randomized.

### Optional Re-Treatment Phase

In the Lutathera arm, patients with radiological progression based on RECIST criteria as per central assessment have the option to enroll for post-progression re-treatment, upon signature of a new informed consent, to receive 2 to 4 additional cycles of 7.4 GBq/200 mCi of Lutathera. If RECIST progression occurs after Week 72 post the primary end point analysis, the decision to enroll the patient in re-treatment will be based on local assessment. Patients who have received Lutathera in Optional Treatment Extension Phase (cross-over) are not eligible for re-treatment with Lutathera.

For enrolment in the optional re-treatment phase, patients must undergo SSTR imaging and meet all requirements.

After enrolment into re-treatment phase, patients will initially receive 2 administrations of Lutathera 7.4 GBq/200 mCi at 8-week interval. After that, the Investigator should determine if:
The patient shows evidence of disease stabilization or response (i.e. absence of radiological progression, or assessed by clinical benefit).
Has shown good tolerance to Lutathera re-treatment.

If the investigator considers this treatment option in the best interest of the participant and if the participant meets these criteria and agrees to continue with further treatment with Lutathera, the Investigator may administer up to 2 additional administrations of Lutathera 7.4 GBq/200 mCi at 8-week interval. A maximum of 4 administrations of Lutathera is allowed during the re-treatment period.

All safety and efficacy assessments in the re-treatment period will be performed locally and following the schedule in initial treatment period (expression of somatostatin receptors on all target lesions will be evaluated locally before re-treatment; there is no central ‘real time’ assessment of the SRI images, however the images should be submitted to the central imaging center possibly within 1 month for a second review).

During the re-treatment phase, the administration of SSAs is at investigator's discretion. The re-treatment phase will last until locally confirmed disease progression or until EoS, whichever occurs first. If a patient discontinues Lutathera before locally confirmed disease progression in re-treatment, the patient should be followed with tumor assessments as per initial CT/MRI schedule until locally confirmed disease progression. After re-treatment phase is complete, patients will be followed up for 3 years or until EoS, whichever occurs first (and at least 6 months after the last re-treatment dose in the study).

The time window to start re-treatment in this study is within 4 years after the last patient randomized.

For patients in Lutathera arm who are eligible for re-treatment beyond this window, Lutathera access for re-treatment may be granted via Post Study Drug Supply (PSDS) programs, based on local regulation.

### Follow Up Phase

At the end of the Treatment Phase (or the optional Treatment Extension Phase in case of cross-over, or the optional Re-treatment Phase) or after discontinuation for any cause (including disease progression), all patients will continue to be followed up at least every 6 months and up to 3 years (or until EoS, whatever comes first) to continue data collection for the secondary endpoints of the study, such as long term safety and overall survival. During the Follow-up Phase, SAEs and adverse events of special interest (AESI) other than secondary hematological malignancies, related to the study treatment and AESI of secondary hematological malignancies irrespective of causality, will be reported. Anti-tumor treatments administered after progression/discontinuation, disease status based on local CT/MRI assessment, and OS data will be collected every 6 months (+1 month) in both arms.

### Rationale

### Rationale for Study Design

This study is justified by the markedly longer PFS and significantly higher response rate in 2^{nd} line G1 and G2 midgut NETs demonstrated in the Lutathera arm of the Phase III NETTER-1 pivotal study (Strosberg, et al., 2017).

The WHO 2010 classification distinguishes G3 NETs (usually ≤55% Ki67) from G3 NECs (usually >55%), recognizing the NET-specific features of G3-NETs (continuum between G2 and G3 NET) (Rindi 2010, Rindi 2014).

While the Erasmus MC Phase I-II study and the NETTER-1 studies have proven the safety and efficacy of Lutathera in well differentiated Grade 1 and 2 GEP-NETs (Kwekkeboom, et al., 2008; Strosberg, et al., 2017), Grade 3 GEP-NETs still represent high unmet medical need for several reasons:
Historically, most studies have excluded G3 NET (˜20% of all NETs);
The number of patients diagnosed at G3 NET stage continues to increase (Dasari, et al., 2017);
There is no universally accepted standard of care for the treatments of G3 NETs. ESMO (Oberg, Knigge, Kwekkeboom, Perren, & Group, 2012), ENET (Perren, et al., 2017), NANETS (Kunz, et al., 2013) and NET NCCN (Kulke, et al., 2015) guidelines outline chemotherapy as primary treatment; however physicians' practice may vary (SSA and/or chemotherapy used in 1^{st} and 2^{nd} line).
Based on experts' considerations and from the NORDIC NEC study, a Ki-67 cutoff between 50% and 60% has been proposed as the minimum level that should be considered to use platinum-based chemotherapy. Therefore, it appears reasonable to manage well-differentiated G 3 and G 2 NET patients similarly (Vélayoudom-Céphise, et al., 2013; Heetfeld, et al., 2015). In addition, prognosis and response rate of well-differentiated G3 NET seem to be close to well differentiated G 1/2 NET with a worse overall survival (Coriat, Walter, Terris, Couvelard, & Ruszniewski, 2016).
Efficacy of current 1^{st} line treatments appear to be modest with low ORR with limited evidence of PFS/OS benefit (Coriat, Walter, Terris, Couvelard, & Ruszniewski, 2016). In addition, in non-pancreatic NET G-3, no chemotherapy regimen should be considered as a standard of first-line care, considering the very small amount of data available.

Therefore, in the current NETTER-2 study an advanced GEP-NET patient population of G2 with Ki67≥10% and G3 with Ki67≤55% has been included to evaluate a potential new treatment option for these patients.

In accordance with the recently finalized guidance for industry on considerations for the inclusion of adolescent patients in adult oncology clinical trials and the consensus expert opinion presented by the European multi-stakeholder platform ACCELERATE to include adolescents in adult oncology clinical trials when the histology and biologic behavior of the cancer under investigation is the same in, or the molecular target of the drug is relevant to, cancers in both adult and adolescent patients (FDA guidance), this study will include adolescents ≥15 years and >40 kg BW. Even though the number of adolescents ≥15 years fulfilling the inclusion criteria for this study is very small, the high unmet medical need in this population is justifying their enrollment. It is not expected that adolescents below the age of 15 years will present with the disease meeting the inclusion criteria for this study. Due to the radioactive nature of Lutathera and its method of infusion, and similarly to the NETTER-1 trial design, it is not possible to implement a fully double-blinded design for this study, however a central, blinded, real-time IRC (Independent Review Committee) assessment is implemented to ensure an independent evaluation of the tumor response according to RECIST 1.1 criteria.

Randomization will be stratified by Grade (G2 vs G3) and tumor origin (pNET vs other origin), preventing imbalance between treatment groups for known factors that influence prognosis. For this trial a 2:1 randomization design is chosen to allow patients to have a higher chance of getting the treatment which is anticipated to be more effective. For the same reason and to minimize the possible higher drop-out rate in the control arm, patients will be offered to cross-over to Lutathera after centrally confirmed RECIST progression (or locally confirmed if it occurs after Week 72 post the primary end point analysis).

Patients randomized in the Lutathera arm will also be offered optional re-treatment with additional 2-4 Lutathera doses/cycles after centrally confirmed RECIST progression (or locally confirmed if it occurs after Week 72 post the primary end point analysis), based on the physician judgment of potential clinical benefit. Overall, therapeutic options in patients who progress after PRRT treatment are limited. Based on a meta-analysis (Strosberg et al., 2021) including patients with progressive GEP-NETs (n=560) who received re-treatment with Lutathera (one to six doses during re-treatment, majority of patients received 200 mCi per administration), the clinical benefit of re-treatment was supported by a median PFS of 12.52 months (available in 414 patients), median OS of 26.78 months from the start of re-treatment (available in 194 patients), and DCR of 71% (available in 347 patients). The safety profile of Lutathera re-treatment was similar to initial treatment. These data indicate that re-treatment with Lutathera may be considered as a treatment approach to maximize benefit without compromising on safety and with a careful assessment of clinical status of each individual patient.

### Rationale for Dose/Regimen and Duration of Treatment

The Lutathera arm dose/regimen of the NETTER-2 study is identical to the established regimen used in the Erasmus MC Phase I/II study (Kwekkeboom, et al., 2008) and in the Phase III NETTER-1 pivotal study (Strosberg, et al., 2017) with Lutathera cumulative dose of 29.6 GBq (800 mCi), divided into 4 administrations every 8+1 weeks, plus octreotide long-acting (30 mg) every 4 weeks.

Lutathera was approved in Europe for the treatment of unresectable or metastatic, progressive, well differentiated (G1 and G2) GEP-NETs and in the USA for the treatment of somatostatin receptor positive GEP-NETs based on the results of the pivotal Phase III NETTER-1 study and data from 1,214 patients with somatostatin receptor-positive tumors, including GEP-NETS, who received Lutathera at the Erasmus Medical Centre in the Netherlands.

Treatment with Lutathera resulted in markedly longer progression-free survival and a significantly higher response rate than high-dose octreotide long-acting among patients with advanced midgut neuroendocrine tumors. Preliminary evidence of an overall survival benefit was seen in an interim analysis; confirmation will be required in the planned final analysis. Clinically significant myelosuppression occurred in less than 10% of patients in the Lutathera group. The PFS-prolongation was similar in the two Ki67 strata (≤2% and 3-20%) suggesting a potential benefit for patients with advanced G2 and 3 GEP-NET to be included in the NETTER-2 study.

Optional re-treatment with additional doses of Lutathera will be offered to patients treated in the Lutathera arm upon disease progression, based on recent published data on re-treatment in GEP-NETs (Strosberg et al., 2021). The candidate patients for re-treatment with Lutathera in this protocol, are patients with centrally documented tumor progression in Lutathera arm, who have completed the 4 doses/cycles of Lutathera during the treatment phase, had CR/PR/SD as best response for at least 6 months (−2 weeks window is allowed) after the 4th Lutathera dose, and had tolerated the treatment (according to criteria defined herein). Patients will be offered to receive initially two Lutathera doses in re-treatment period. Based on the physician judgment of clinical benefit derived from the first 2 doses, patients may receive up to 2 additional doses of Lutathera (criteria for additional doses are listed herein). A maximum of 4 doses of Lutathera is allowed during the re-treatment period. The dose level assessed in the re-treatment portion of this study will be the same as the approved level of 7.4 GBq per cycle, based on the published meta-analysis data that showed that majority of patients received this dose (Strosberg et al., 2021).

### Rationale for Choice of Control Drug

The control arm dose/regimen of the NETTER-2 study is identical to the dose/regimen used in the NETTER-1 study (octreotide long-acting (60 mg) every 4 weeks).

The clinical experience with octreotide is extensive, but there is currently a lack of data in the use of somatostatin analogues in G3 GEP-NET patients.

Patients in the comparator arm of the present NETTER-2 study will receive 60 mg octreotide long-acting at 4-weeks intervals. The dose is supported by findings of Broder et al. (2015), who performed a systematic review of the literature to analyze the anti-proliferative benefit of somatostatin analogs. They confirmed that doses of >30 mg octreotide long-acting are commonly used by clinical experts for symptom and tumor progression control in NET patients. This dose (60 mg) was also tested in comparator arm of the pivotal Phase III NETTER-1 study (Strosberg, et al., 2017). In current clinical practice, it is likely that even a higher percentage of patients receive >30 mg of Octreotide LAR (Anthony & Vinik, 2011; Joseph, et al., 2010; Wolin, 2012; Broder, Beenhouwer, Strosberg, Neary, & Cherepanov, 2015). The 4-week interval injections of 60 mg octreotide long-acting is a higher dose than the 4-week interval injections with 20 mg or 30 mg, which is presently the registered dose for Sandostatin® LAR Depot. With this treatment, the majority of symptomatic patients show an improvement in QoL, and most patients obtain temporary stable disease based on CT scans (Faiss, et al., 1999; Faiss, et al., 2003; Rinke, et al., 2009; Ludlam & Anthony, 2011; Anthony & Vinik, 2011; Wolin, 2012; Broder, Beenhouwer, Strosberg, Neary, & Cherepanov, 2015). Furthermore, according to the Phase III NETTER-1 study results, patients with metastatic midgut tumors progressive after standard dose (30 mg) SSAs treated in the control arm of the study with high dose (60 mg) octreotide long-acting had a median time free from relapse of 8.4 months (Strosberg, et al., 2017) without notable side effects, confirming that this regimen has anti-tumor benefit and is well tolerated.

In a clinical study published by Astruc et al. (Astruc, et al., 2005), in which healthy subjects were treated with 20 mg and 60 mg of Octreotide LAR, the PK data showed that the plasma exposure was dose-proportional and the treatment was well tolerated at both doses. Available pharmacokinetic (PK) data in children at 40 mg Octreotide LAR show that the plasma exposure, corrected for the dose, is similar in adults and young patients. Moreover, Octreotide LAR was well tolerated in children at 40 mg. It should be noted that the same dose was given to <12 and >12 years old patients.

Therefore, in accordance with available PK data and in line with FDA guidance for inclusion of adolescents in the adult oncology trials and the consensus expert opinion presented by the European multi-stakeholder platform ACCELERATE it is considered acceptable to include adolescents in the NETTER-2 study at the same fixed dose administered in adults, considering a population ≥15 y and >40 kg.

Based on currently available data of high dose somatostatin analogues, the use as a comparator in this study, at 60 mg dosage, is justified considering the high unmet medical need in a population not candidate for chemo or targeted therapy in the investigator opinion.

### Purpose and Timing of Interim Analyses

At the time of the PFS primary analysis, an estimate of overall survival will be calculated in terms of hazard ratio (point estimate) and 95% confidence interval.

Risks and benefits Neuroendocrine tumors (NETs) represent a small proportion of cancers but are increasing in incidence due to incidental diagnosis. Prognosis for grade 1 and localized NETs has steadily improved, however patients with distant and/or grade 3 NETs continue to fare poorly (Sackstein, O'Neil, Neugut, Chabot, & Fojo, 2018).

Patient management poses a significant challenge because of the heterogeneous clinical presentations and varying degrees of aggressiveness.

Randomization will be stratified by tumor origin (pNET vs other origin) and Grade (G2 vs G3), preventing imbalance between treatment groups for known factors that influence prognosis.

The population of G2 and G3 GEP-NET patients included in the NETTER-2 study is not candidate for chemo or targeted therapy in the investigator opinion. G3 GEP-NET patients with Ki67≤55 are a category of G3 neuroendocrine neoplasm (NEN) where tumors retain their well-differentiated characteristics, continue to express somatostatin receptors, but have a higher proliferation rate then the majority of GEP-NETs (Rindi, et al., 2018). Aggressive G2 GEP-NETs patients with Ki67 range 10% to 20% share with G3 GEP-NETs the greater likelihood of poor prognosis, and limited treatment options. Despite advances in medical therapy for these tumors, there is still a high unmet medical need to control tumor growth, especially for G3 GEP-NETs (˜10-20% of all NETs), which historically have been excluded from most studies (Sorbye, et al., 2019; Coriat, Walter, Terris, Couvelard, & Ruszniewski, 2016; Dasari, et al., 2017).

The most important risk mitigating factor for GEP-NET patients who are considered for treatment with Lutathera, is the ability to use receptor scintigraphy imaging to select those patients who are most likely to respond to PRRT, and to exclude patients who would not benefit from the treatment because their tumors do not express high enough levels of the somatostatin receptor.

Based on the results achieved in other PRRT studies using ¹⁷⁷Lu-dotatate (Strosberg 2017; 2018 Brabander 2016), the major expected benefits to patients treated with Lutathera include the high probability of extending progression free survival, increasing overall survival and increasing time to deterioration of quality of life.

For this trial a 2:1 randomization design is chosen to allow patients to have a higher chance of getting the treatment which is anticipated to be more effective. For the same reason and to minimize the possible higher drop-out rate in the control arm, patients will be offered to cross-over to Lutathera after centrally confirmed RECIST progression (or locally confirmed if it occurs after Week 72 post the primary end point analysis).

The highest risks arising from treatment with Lutathera are radiation toxicities affecting either bone marrow or kidney function (Bergsma, et al., 2016a; Bergsma, et al., 2016b). Kidney function risks are largely eliminated by the co-infusion of 2.5% Lys-Arg solutions during administration of Lutathera, which reduces the radiation dose to the kidney by approximately 45% (Rolleman, Valkema, Jong, Kooij, & Krenning, 2003). In the NETTER-1 trial no evidence of renal toxicities occurred within the period of observation (Strosberg, et al., 2017).

Bone marrow toxicity may occur in two forms: 1) acute toxicity that occurs after administration of Lutathera, during the treatment period; and 2) delayed toxicity that occurs in 1.5 to 2% of patients. The potential observed effects of short-term bone marrow toxicity are anemia, thrombocytopenia, and neutropenia which are usually mild and transient. In the NETTER-1 trial, grade 3 or 4 neutropenia, thrombocytopenia, and lymphopenia occurred in 1%, 2%, and 9% of patients (Strosberg, et al., 2017). Where hematological toxicity occurs, a trend towards stabilization followed by improvement in patients with longer follow-up is observed. It is important to note that the lymphocyte toxicity observed following PRRT is not a major concern regarding risk of infections, since only B lymphocytes are affected, a subtype which is not directly involved in infection defense (Sierra, et al., 2009).

There is evidence that males may be at risk of decreased spermatogenesis following PRRT with ¹⁷⁷Lu-dotatate. In a study by (Teunissen, et al., 2009) (Teunissen, et al., 2009) a significant decrease of mean serum inhibin B levels was observed in male patients (N=35) treated with ¹⁷⁷Lu-dotatate. It has been demonstrated that serum inhibin B levels are positively correlated with spermatogenic status and sperm count (Pierik, Vreeburg, Stijnen, De Jong, & Weber, 1998) (Pierik, Vreeburg, Stijnen, De Jong, & Weber, 1998). The study (Teunissen, et al., 2009) (Teunissen, et al., 2009) also found recovery to almost pretreatment levels after 24 months. The potential long-term genetic damage to spermatogenic cells has not been studied.

Women and men should not procreate until six months after the end of their last treatment with Lutathera. However, due to additional exposure from CT scans taken during the study, women in both arms should also not procreate during the whole treatment period of the study. Women of child bearing potential and sexually active males must be informed that taking the study treatment may involve unknown risks to the fetus if pregnancy were to occur during the study, and agree that in order to participate in the study they must adhere to the contraception requirements outlined in the exclusion criteria. If there is any question that the subject will not reliably comply, they should not be entered or continue in the study. Based on the FDA guidance for inclusion of adolescents in the adult oncology trials and the consensus expert opinion presented by the European multi-stakeholder platform

ACCELERATE it is recommended that adolescents can receive the same dose administered in adults if there is no relevant effect of body size on pharmacokinetics, which is the case for both, Lutathera and octreotide long-acting. Based on the similar clinical presentation of the disease under evaluation in adolescents, the following inclusion criteria have been chosen for this study: ≥15 years old and >40 kg BW (aligned with the BW recommendation from the FDA guidance).

With regards to re-treatment with Lutathera, the results of a meta-analysis of patients with progressive GEP-NETs who received re-treatment with peptide receptor radionuclide therapy (PRRT), are supportive of administration of additional Lutathera doses after progression on initial treatment. It was shown that the safety profile of Lutathera re-treatment was similar to initial treatment (Strosberg et al., 2021). In this meta-analysis, additional treatment courses with Lutathera ranged from one to six doses, and majority of patients received 200 mCi per administration. Hematologic grade 3/4 AEs were reported in 9% of patients who received PRRT re-treatment. Notably, AML and MDS occurred in <1% of patients who received re-treatment with Lutathera, comparable to the incidence observed with initial PRRT. Re-treatment with Lutathera provided encouraging median PFS. This data indicates that re-treatment with Lutathera can be offered to patients to maximize benefit without compromising on safety and with a careful assessment of clinical status of each individual patient. Dose modifying toxicity rules have been implemented in this study taking into account the higher GEP-NET severity in the NETTER-2 study compared to the approved indication. The administration of a full Lutathera dose (200 mCi/7.4 GBq) if a toxicity is resolved will maximize the potential treatment effects and maintain a positive benefit/risk ratio. To ensure the safety of the subjects in this trial, appropriate eligibility criteria and study procedures, as well as close clinical monitoring, following dose modifying toxicity rules, and Steering Committee study oversight are included in this protocol.

In light of all the above-mentioned elements, the benefit-risk balance of Lutathera within the overall clinical context defined in the clinical trial appears to weight in favor of patient's benefit.

### Population

In this study, safety and efficacy of treatment with Lutathera plus octreotide long-acting (30 mg) versus high dose octreotide long-acting (60 mg) is evaluated in patients ≥15 years old with somatostatin receptor positive, well differentiated G2 (Ki67 index ≥10%) and G3 (Ki67≤55%) advanced GEP-NETs. Patients with documented RECIST progression to previous treatments for the current GEP-NET at any time prior to randomization are not eligible to participate in this study.

### Inclusion Criteria

Subjects eligible for inclusion in this study must meet all of the following criteria:
1. Presence of metastasized or locally advanced, inoperable (curative intent) histologically proven, well differentiated Grade 2 or Grade 3 gastroenteropancreatic neuroendocrine (GEP-NET) tumor diagnosed within 6 months prior to screening.
2. Ki67 index ≥10 and ≤55%.
3. Patients ≥15 years of age and a body weight of >40 kg at screening.
4. Expression of somatostatin receptors on all target lesions documented by CT/MRI scans, assessed by any of the following somatostatin receptor imaging (SRI) modalities within 3 months prior to randomization: [68Ga]-DOTA-TOC (e.g. Somakit-TOC®) PET/CT (or MRI when applicable based on target lesions) imaging, [68Ga]-DOTA-TATE PET/CT (or MRI when applicable based on target lesions) imaging (e.g. NETSPOT®), Somatostatin Receptor scintigraphy (SRS) with [111In]-pentetreotide (Octreoscan® SPECT/CT), SRS with [99mTc]-Tektrotyd, [64Cu]-DOTA-TATE PET/CT (or MRI when applicable based on target lesions) imaging.
5. The tumor uptake observed in the target lesions must be >normal liver uptake.
6. Karnofsky Performance Score (KPS) ≥60.
7. Presence of at least 1 measurable site of disease.
8. Patients who have provided a signed informed consent form to participate in the study, obtained prior to the start of any protocol related activities

### Exclusion Criteria

Subjects meeting any of the following criteria are not eligible for inclusion in this study.
1. Creatinine clearance <40 mL/min calculated by the Cockroft Gault method.
2. Hb concentration <5.0 mmol/L (<8.0 g/dL); WBC <2×10⁹/L (2000/mm³); platelets <75×10⁹/L (75×10³/mm³).
3. Total bilirubin >3×ULN.
4. Serum albumin <3.0 g/dL unless prothrombin time is within the normal range.
5. Pregnancy or lactation.
   A) Women of child-bearing potential, defined as all women physiologically capable of becoming pregnant, are not allowed to participate in this study UNLESS they are using highly effective methods of contraception throughout the study treatment period (including cross-over and re-treatment, if applicable) and for 7 months after study drug discontinuation. Highly effective contraception methods include:
      True abstinence when this is in line with the preferred and usual lifestyle of the patient. Periodic abstinence (e.g., calendar, ovulation, symptothermal, post-ovulation methods), declaration of abstinence for the duration of exposure to IMP, and withdrawal are not acceptable methods of contraception.
      Male or female sterilization
      Combination of any two of the following (a+b or a+c or b+c):
   a. Use of oral, injected, or implanted hormonal methods of contraception. In case of use of oral contraception, women should be stable on the same pill for a minimum of 3 months before taking study treatment.
   b. Placement of an intrauterine device (IUD) or intrauterine system (IUS)
   c. Barrier methods of contraception: condom or occlusive cap (diaphragm or cervical/vault caps) with spermicidal foam/gel/film/cream/vaginal suppository. Post-menopausal women are allowed to participate in this study. Women are considered post-menopausal and not of child bearing potential if they have had 12 months of natural (spontaneous) amenorrhea with an appropriate clinical profile (e.g. age appropriate, history of vasomotor symptoms) confirmed by a high follicle stimulating hormone (FSH) level, or have had surgical bilateral oophorectomy (with or without hysterectomy) or tubal ligation at least six weeks prior to screening. In the case of oophorectomy alone, only when the reproductive status of the woman has been confirmed by follow up hormone level assessment is she considered not of child bearing potential.
   B) Sexually active male patients, unless they agree to remain abstinent (refrain from heterosexual intercourse) or be willing to use condoms and highly effective methods of contraception with female partners of childbearing potential or pregnant female partners during the treatment period (including cross-over and re-treatment, if applicable) and for 4 months after study drug discontinuation. In addition, male patients must refrain from donating sperm during this same period.
6. Peptide receptor radionuclide therapy (PRRT) at any time prior to randomization in the study.
7. Documented RECIST progression to previous treatments for the current GEP-NET at any time prior to randomization.
8. Patients for whom in the opinion of the investigator other therapeutic options (eg chemo-, targeted therapy) are considered more appropriate than the therapy offered in the study, based on patient and disease characteristics.
9. Any previous therapy with Interferons, Everolimus (mTOR-inhibitors), chemotherapy or other systemic therapies of GEP-NET administered for more than 1 month or within 12 weeks prior to randomization in the study.
10. Any previous radioembolization, chemoembolization and radiofrequency ablation for GEP-NET.
11. Any surgery within 12 weeks prior to randomization in the study.
12. Known brain metastases, unless these metastases have been treated and stabilized for at least 24 weeks, prior to screening in the study. Patients with a history of brain metastases must have a head CT or MRI with contrast to document stable disease prior to randomization in the study.
13. Uncontrolled congestive heart failure (NYHA II, III, IV). Patients with history of congestive heart failure who do not violate this exclusion criterion will undergo an evaluation of their cardiac ejection fraction prior to randomization via echocardiography. The results from an earlier assessment (not exceeding 30 days prior to randomization) may substitute the evaluation at the discretion of the Investigator, if no clinical worsening is noted. The patient's measured cardiac ejection fraction in these patients must be ≥40% before randomization.
14. QTcF>470 msec for females and QTcF>450 msec for males or congenital long QT syndrome.
15. Uncontrolled diabetes mellitus as defined by hemoglobin A1c value >7.5%.
16. Hyperkaleamia >6.0 mmol/L (CTCAE Grade 3) which is not corrected prior to study enrolment.
17. Any patient receiving treatment with short-acting octreotide, which cannot be interrupted for 24 h before and 24 h after the administration of Lutathera, or any patient receiving treatment with SSAs (eg octreotide long-acting), which cannot be interrupted for at least 6 weeks before the administration of Lutathera.
18. Patients with any other significant medical, psychiatric, or surgical condition, currently uncontrolled by treatment, which may interfere with the completion of the study.
19. Prior external beam radiation therapy to more than 25% of the bone marrow.
20. Current spontaneous urinary incontinence.
21. Other known co-existing malignancies except non-melanoma skin cancer and carcinoma in situ of the uterine cervix, unless definitively treated and proven no evidence of recurrence for 5 years.
22. Patient with known incompatibility to CT Scans with I.V. contrast due to allergic reaction or renal insufficiency. If such a patient can be imaged with MRI, then the patient would not be excluded.
23. Hypersensitivity to any somatostatin analogues, the IMPs active substance or to any of the excipients.
24. Patients who have participated in any therapeutic clinical study/received any investigational agent within the last 30 days.

### Eligibility Criteria for Optional Treatment Extension Phase (Cross-Over)

Any centrally confirmed RECIST progressive patient has the option to enroll for post-progression cross-over and receive maximum 4 cycles of Lutathera (7.4 GBq/200 mCi×4 cycles; cumulative dose: 29.6 GBq/800 mCi) plus octreotide long-acting (30 mg every 8 weeks), provided that the following criteria are met:
Patients have provided a signed informed consent form to participate in the study treatment extension phase, obtained prior to the start of any related procedures.
Expression of somatostatin receptors on all target lesions (based on post-progression new baseline assessment) documented by CT/MRI scans (Note: Cross-over Eligibility CT/MRI scan doesn't need to be repeated if performed within the previous 12 weeks), assessed by any of the following somatostatin receptor imaging (SRI) modalities within 3 months prior to Lutathera treatment: [68Ga]-DOTA-TOC (e.g. Somakit-TOC®) PET/CT imaging (or MRI when applicable based on target lesions), [68Ga]-DOTA-TATE PET/CT (or MRI when applicable based on target lesions) imaging (e.g. NETSPOT®), Somatostatin Receptor scintigraphy (SRS) with [111 In]-pentetreotide (Octreoscan® SPECT/CT), SRS with [99mTc]-Tektrotyd, [64Cu]-DOTA-TATE PET/CT imaging (or MRI when applicable based on target lesions).
All protocol inclusion/exclusion criteria are met.
The time window to start cross-over is 4 years after last patient randomized.
Note: Prior each Lutathera administration a 6-week washout period from last octreotide long-acting administration has to be applied.

If RECIST progression occurs after Week 72 post the primary end point analysis, the decision to enroll the patient in cross-over will be based on local assessment.

Images (Cross-over Eligibility SRI and CT or MRI scan) will be also submitted to the central imaging center for a second review: there is no central ‘real time’ assessment of the Cross-Over Eligibility SRI uptake; however, the images should be submitted to the central imaging center possibly within 1 month. Images submission is not required for screening failure patients.

### Eligibility Criteria for Optional Re-Treatment Phase

In the Lutathera arm, patients with radiological progression based on RECIST criteria in central assessment, have the option to enroll for post-progression re-treatment, upon signature of a new informed consent, to receive 2 to 4 additional cycles of 7.4 GBq/200 mCi of Lutathera.

For enrolment in the optional re-treatment phase, patients must meet all following requirements:
Randomized to Lutathera arm, received 4 doses of Lutathera in initial treatment and experienced either disease stabilization (SD) or objective response (PR/CR) (centrally confirmed) for at least 6 months after the 4th Lutathera dose. If a patient's response has changed from PR/CR to SD within 6 months after the 4th Lutathera dose, the patient is still eligible for re-treatment, provided there is no documented progression within 6 months. A window of-2 weeks is allowed for the confirmation of 6 months of SD/PR/CR response.
Lutathera was overall well tolerated; no recorded SAEs related to Lutathera that were not resolved before the next Lutathera dose and led to treatment interruption.
After the SD/PR/CR response, experienced radiological RECIST progression (centrally confirmed). If RECIST progression occurs after Week 72 post the primary end point analysis, the decision to enroll the patient in re-treatment will be based on local assessment.
Patient did not receive any other systemic treatment for GEP-NET after progression (somatostatin analogues are allowed).
Investigator's agreement that re-treatment is in the best interest for the patient
Patients have provided a signed informed consent form to participate in the re-treatment phase, obtained prior to the start of any related procedures.
Time window to start re-treatment: 4 years after last patient randomized.
Expression of somatostatin receptors on all target lesions (based on post-progression new baseline assessment) documented by CT/MRI scans (Note: Re-treatment Eligibility CT/MRI scan doesn't need to be repeated if performed within the previous 12 weeks), assessed by any of the following somatostatin receptor imaging (SRI) modalities within 3 months prior to Lutathera re-treatment: [68Ga]-DOTA-TOC (e.g. Somakit-TOC®) PET/CT imaging (or MRI when applicable based on target lesions), [68Ga]-DOTA-TATE PET/CT (or MRI when applicable based on target lesions) imaging (e.g. NETSPOT®), Somatostatin Receptor scintigraphy (SRS) with [111In]-pentetreotide (Octreoscan® SPECT/CT), SRS with [99mTc]-Tektrotyd, [64Cu]-DOTA-TATE PET/CT imaging (or MRI when applicable based on target lesions).
Images (Re-treatment Eligibility SRI and CT or MRI scan) will be also submitted to the central imaging center for a second review: there is no central ‘real time’ assessment of the Re-treatment Eligibility SRI uptake, however the images should be submitted to the central imaging center possibly within 1 month. Images submission is not required for screening failure patients.

### Treatment

In this study, approximately 222 patients with advanced G2-3 GEP-NET will be randomized (2:1 randomization ratio) to receive treatment with Lutathera (7.4 GBq or 200 mCi x 4 administrations every 8±1 weeks; cumulative dose: 29.6 GBq or 800 mCi) plus octreotide long-acting standard dose (30 mg every 8 weeks during Lutathera treatment and every 4 weeks after last Lutathera treatment) or octreotide long-acting high dose (60 mg every 4 weeks).

The investigational drug product Lutathera® (¹⁷⁷Lu-DOTA⁰-Tyr³-Octreotate) will be provided by the Sponsor. The Sponsor will also provide the 2.5% Lys-Arg sterile amino acid solution for infusion (if it can't be compounded at the hospital Pharmacy), as well as octreotide long-acting (Sandostatin® LAR Depot) for the entire duration of the Treatment Phase (and optional Treatment Extension Phase) of the study. For the optional Re-treatment Phase, the Sponsor will provide Lutathera and 2.5% Lys-Arg sterile amino acid solution; octreotide-long acting administration is not mandatory and is at investigator discretion. Patients will switch to prescribed drugs in the follow up phase.

Anti-emetics, SRI imaging agents, short-acting octreotide or any other supportive care medication will not be supplied by the Sponsor.

### Study Treatments

### Investigational and Control Drugs

The investigational and control drugs supplied by Sponsor are listed in the following table.

### Investigational and Control Drug

| Investigational/ | | | | | |
|---|---|---|---|---|---|
| Control Drug | | | | | |
| (Name and | Pharmaceutical | Route of | Supply | | Study |
| Strength) | Dosage Form | Administration | type | Supplier | arm |
| Lutathera ® | Radiopharmaceutical | Intravenous | Open | Sponsor | Lutathera |
| | solution for infusion | use | label; | | |
| | (7.4 GBq of | | vials | | |
| | Lutathera per 30 ml | | | | |
| | vial) | | | | |
| 2.5% Lys-Arg | 25 g L-lysine-HCl and | Intravenous | Open | Sponsor* | Lutathera |
| sterile amino | 25 g L-arginine-HCl | use | label; | | |
| acid solution | in 1000 mL | | flex | | |
| | | | bags | | |
| Octreotide long- | For injectable | Intragluteal | Open | Sponsor | Lutathera; |
| acting | suspension: strengths | injection | label, | | Control |
| (Sandostatin | 10 mg per 6 mL, 20 | | vials | | |
| LAR ® Depot) | mg per 6 mL, or 30 | | | | |
| | mg per 6 mL vials | | | | |

*Sponsor will provide the 2.5% Lys-Arg sterile amino acid solution for infusion (if it can't be compounded at the hospital Pharmacy).

### Lutathera Arm

### Investigational Drug Product: Lutathera® (¹⁷⁷Lu-Dotatate)

Lutathera is a sterile radiopharmaceutical supplied as a ready-to-use solution for infusion containing ¹⁷⁷Lu-DOTA⁰-Tyr³-Octreotate as Drug Substance with a volumetric activity of 370 MBq/mL at reference date and time (calibration time (tc)).

On the day of Lutathera treatment, an intravenous bolus of anti-emetic is given. For renal protection, a sterile amino acids solution is infused 30 minutes before the start of Lutathera, and continues for a total of 4 h.

Lutathera is a radiopharmaceutical solution for infusion supplied as a ready-to-use product. The only Quality Control (QC) tests that must be performed at the clinical site are; 1) confirm correct product certificate; 2) determine total radioactivity; 3) confirm visual appearance. Since Lutathera is manufactured in centralized GMP facilities, the majority of required QC tests are performed before product shipment. A batch release certificate of the product will be sent to the investigational centres. This batch release certificate is provided by the Qualified Person (QP) of the manufacturing site to ensure that the product is suitable for administration and that it meets the specifications indicated in the product leaflet. Manufacturing site prepares single doses calibrated within the range of 7.4 GBq±10% (200 mCi) between t0+6 h and t0+52 h after the end of production. Certificate of release reports both the exact activity provided and the time when this activity is reached. The total amount of radioactivity per single dose vial is 7,400 MBq at the date and time of infusion. Given the fixed volumetric activity of 370 MBq/ml at the date and time of calibration, the volume of the solution is adjusted between 20.5 ml and 25.0 ml in order to provide the required amount of radioactivity at the date and time of infusion.

The composition of the drug product is listed in the table below.

### Composition of the Lutathera® Drug Product (Per Vial)

| | Content | | | | | |
|---|---|---|---|---|---|---|
| | (Unit and/or percentage) | | | | | |
| | V = 20.5 mL | | V = 25.0 mL | | | |
| | (min | | (max | | | Quality |
| Component | volume) | | volume) | | Function | standards |
| Drug Substance: | 370 MBq/mL ± | | | | Drug | — |
| ¹⁷⁷Lu-DOTA⁰-Tyr³- | 5% at Tc | | | | Substance | |
| Octreotate (volumetric | (EoP) | | | | | |
| activity) | | | | | | |
| Activity at Injection time | 7.4 GBq ± 10% | | | | — | — |
| ¹⁷⁶⁺¹⁷⁷Lu-DOTA⁰-Tyr³- | 93.7 | μg | 114.2 | μg | — | NA¹ |
| Octreotate² | | | | | | |
| DOTA-Tyr³-Octreotate | 121.3 | μg | 148.0 | μg | Chemical | — |
| | | | | | precursor | |
| Excipients* | | | | | | |
| Acetic acid | 9.8 | mg | 12.0 | mg | pH adjuster | In-house |
| Sodium acetate | 13.5 | mg | 16.5 | mg | pH adjuster | In-house |
| Gentisic acid | 12.9 | mg | 15.8 | mg | RSE | In-house |
| Ascorbic acid | 57.3 | mg | 69.9 | mg | RSE | In-house |
| Diethylene triamine | 1.0 | mg | 1.3 | mg | Sequestering | In-house |
| pentaacetic acid (DTPA) | | | | | agent | |
| Sodium chloride (NaCl) | 140.4 | mg | 171.0 | mg | Isotonizing | BP monograph |
| | | | | | agent | “Sodium Chloride |
| | | | | | | infusion“ |
| Sodium hydroxyde | 13.1 | mg | 16.0 | mg | pH adjuster | Ph. Eur. 0677 |
| (NaOH) | | | | | | |

| | | | | |
|---|---|---|---|---|
| Water for injection | Ad 20.5 | Ad 25.0 | Solvent | Ph. Eur. 0169/ |
| | mL** | mL** | | USP |

Tc = calibration time = EOP: End of Production = t₀ = activity measurement of the first vial
RSE: Radiation Stability Enhancer
¹The synthesis of the Drug Substance and formulation into the Drug Product are parts of a continuous process and therefore the Drug Substance is not isolated
²¹⁷⁶⁺¹⁷⁷Lu-DOTA⁰-Tyr³-Octreotate represents the fraction of peptide labeled to lutetium (radioactive and non-radioactive lutetium) and corresponds to the sum of ¹⁷⁶Lu-DOTA⁰-Tyr³-Octreotate and ¹⁷⁷Lu-DOTA⁰-Tyr³-Octreotate
*Indicated values calculated at calibration times based on ¹⁷⁷Lu specific activity of 740 GBq/mL (20 Ci/mg) at labeling time and a mean synthesis yield of 80% and radiochemical purity ≥97%.
** This amount includes as well the water for injection filled in the bulk solution bottle (0.5 ± 0.1 mL) and in the primary packaging (0.20 ± 0.02 mL) during the sterilization process as added amount is considered negligible.

### Concomitant Treatment: 2.5% Lys-Arg Sterile Amino Acid Solution

In the present study patients randomized in Lutathera arm will receive concomitant 2.5% Lys-Arg solution for kidney protection (see following table). For renal protection purpose, the 2.5% Lys-Arg solution must be administered intravenously for 4 hours (infusion rate: 250 ml/h); the infusion should start 30 minutes prior to the start of the Lutathera infusion, and continue during and up to at least 3 hours after the Lutathera infusion.

Hyperkaleamia must be corrected prior to 2.5% Lys-Arg infusion if >6.0 mmol/L (CTCAE Grade 3).

The 2.5% Lys-Arg solution will be supplied by Sponsor if it can't be compounded at the hospital Pharmacy. The composition of the 2.5% Lys-Arg solution is shown in the table below.

2.5% Lys-Arg sterile solution composition*

| Component | Quantity/1000 ml |
|---|---|
| L-lysine HCL | 25 g |
| L-arginine HCl | 25 g |
| Water for injection | qs 1000 ml |

*The recommended pH range of the 2.5% Lys-Arg sterile solution is 5.0-7.0 with an osmolarity of 420-480 mOsm/kg.

In addition to the antiemetic premedication, in case of persistent nausea or vomiting during the administration of amino acids, Investigators are advised to use antiemetics which are commonly prescribed in their institutions for treatment of nausea induced by chemotherapeutic drugs. Among such antiemetics, the use of Aprepitant (Emend®) should be considered. Haloperidol (Haldol®) could also be considered as an adjunct treatment (either i.v. or oral) in case the advised antiemetic regimens are not successful and patients continue to vomit as well as Lorazepam (Ativan®).

In case where the treatments previously provided for nausea and vomiting are insufficient, a single dose of corticosteroids can be used, as long as it is not given before initiating or within one hour after the end of Lutathera infusion.

Concomitant Treatment: 30 mg octreotide long-acting (Sandostatin® LAR Depot) Sandostatin® LAR Depot (octreotide long-acting) is a pharmaceutical that is available in a single-use kit containing a 6-mL vial of 10 mg, 20 mg, or 30 mg strength for intramuscular injection, a syringe containing 2.5 mL of diluent, two sterile 1½″ 19 gauge needles, and two alcohol wipes. For prolonged storage, Sandostatin® LAR Depot should be stored at refrigerated temperatures between 2° C. and 8° C. (36° F.-46° F.) and protected from light until the time of use. An instruction booklet for the preparation of drug suspension for injection is also included with each kit.

Octreotide long-acting 30 mg (Sandostatin® LAR Depot) is preferably administered the day after each administration of Lutathera and no earlier than 4 hours after completion of the Lutathera infusion. Due to the 6-weeks washout period before each Lutathera injection, no additional octreotide long-acting administrations are recommended between Lutathera treatments. Once Lutathera treatment completed, and also in case the Lutathera infusions have been suspended (e.g due to Dose Modifying Toxicity), patients will continue the 4-week interval administrations of 30 mg octreotide long-acting until the completion of the Treatment Phase.

### Control Arm

### 60 mg Octreotide Long-Acting (Sandostatin® LAR Depot)

Sandostatin® LAR Depot (octreotide long-acting) is a pharmaceutical that is available in single-use kits containing a 6-mL vial of 10 mg, 20 mg, or 30 mg strength for intramuscular injection, a syringe containing 2.5 mL of diluent, two sterile 1½″ 19 gauge needles, and two alcohol wipes. For prolonged storage, Sandostatin® LAR Depot should be stored at refrigerated temperatures between 2° C. and 8° C. (36° F.-46° F.) and protected from light until the time of use. An instruction booklet for the preparation of drug suspension for injection is also included with each kit.

In the control arm, patients will receive administrations of octreotide long-acting 60 mg (Sandostatin® LAR Depot) at 4-week intervals (+/−3 days) until the completion of the Treatment Phase (i.e., two injections of Sandostatin® LAR 30 mg per treatment), unless the patient progresses or dies.

Patients who were SSA-naïve before enrolment in the study, should receive first planned dose at 30 mg and the second (and subsequent) planned dose(s) at 60 mg (full dose).

### Other Treatment(s)

### Rescue Medication

### Short-Acting OCTREOTIDE

Subcutaneous, short-acting octreotide injections may be indicated for control of symptoms (i.e. diarrhoea and flushing) in patients in both study arms, in accordance with the manufacturer's prescribing information. Short-acting octreotide for symptom control is administered by the patient (at home) at investigator's discretion.

### Other Anti-Cancer Treatments

The patient may not receive any other systemic therapy for the treatment of GEP-NET (chemotherapeutic, biologic, or any investigational agent) other than Lutathera and/or short-acting octreotide and/or octreotide long-acting during the study Treatment Phase. Localized therapy such as surgery or external beam irradiation may be performed on additional site(s), provided that it does not affect treatment response assessment; no surgeries are allowed within 12 weeks prior to Lutathera administration.

The last administration of long-acting SSA before the start of the study treatment is allowed during the screening period, before randomization, but should be carefully planned to allow a 6 weeks washout period prior to the administration of Lutathera.

New anti-cancer treatments administered after progression or during the Follow up Phase must be registered in the eCRF.

### Prohibited Medication

Somatostatin and its analogues competitively bind to somatostatin receptors. Therefore, administration of long-acting somatostatin analogues should be avoided within 6 weeks prior to the administration of Lutathera. If necessary, patients may be treated with short acting somatostatin analogues during the 6 weeks until 24 hours preceding Lutathera administration.

There is some evidence that corticosteroids can induce down-regulation of SST2 receptors. Therefore, as a matter of caution, repeated administration of high-doses of glucocorticosteroids should be avoided during Lutathera treatment. Patients with a history of chronic use of glucocorticosteroids should be carefully evaluated for sufficient somatostatin receptor expression. It is not known if there is any interaction between glucocorticosteroids used intermittently for the prevention of nausea and vomiting during Lutathera administration. Therefore, glucocorticosteroids should be avoided as preventive anti-emetic treatment. In the case where the treatments previously provided for nausea and vomiting are insufficient, a single dose of corticosteroids can be used, as long as it is not given before initiating or within one hour after the end of Lutathera infusion.

The absence of inhibition or significant induction of the human CYP450 enzymes, the absence of specific interaction with P-glycoprotein (efflux transporter) as well as OAT1, OAT3, OCT1, OCT2, OATP1B1, OATP1B3 and BCRP transporters in pre-clinical studies suggest that Lutathera has a low probability of causing clinically relevant metabolism- or transporter-mediated interactions.

The following cautions regarding possible Octreotide Drug-Drug-Interactions (DDI) must be observed (according to the Octreotide label):
Dose adjustment of medicinal products such as beta blockers, calcium channel blockers, or agents to control fluid and electrolyte balance may be necessary when administered concomitantly.
Dose adjustments of insulin and antidiabetic medicinal products may be required when administered concomitantly.
Octreotide has been found to reduce the intestinal absorption of ciclosporin and to delay that of cimetidine.
Concomitant administration of octreotide and bromocriptine increases the bioavailability of bromocriptine.
Drugs mainly metabolized by CYP3A4 and which have a low therapeutic index should be used with caution (e.g. quinidine, terfenadine).
Octreotide may alter absorption of dietary fats in some patients.
Depressed vitamin B12 levels and abnormal Schilling's tests have been observed in some patients receiving octreotide therapy. Monitoring of vitamin B12 levels is recommended during therapy with Octreotide LAR in patients.

### Subject Numbering, Treatment Assignment, Randomization

### Subject Numbering

For all patients who have signed the ICF, a screening number will be assigned in chronological order starting with the lowest number available on site. Patients will be identified by a unique patient identification number (Patient ID No.) composed of the center number and the screening number.

### Treatment Assignment, Randomization

After the screening period, eligible patients will be randomly assigned (ratio 2:1) to one of the two study groups for treatment with Lutathera plus octreotide long-acting (30 mg) or high dose octreotide long-acting (60 mg). The randomization system will assign a unique randomization number to the patient, which will be used to link the patient to a treatment arm. Randomization will be stratified by tumor Grade (G2 vs G3) and tumor origin (pNET vs other origin) according to a stratified permuted block scheme.

The details of the procedure to obtain the patient randomization number will be described in the Investigator's Manual.

### Treatment Blinding

Due to the radioactive nature of Lutathera and its method of infusion, it is not possible to implement a blinded design for this study.

### Dose Compliance, Modification and Discontinuation

### Dose Compliance

For each single-dose of Lutathera a deviation of ±10% from the scheduled dose is allowed.

### Dose Modification

Modifying Toxicities (DMT) will apply in both arms. Patients whose treatment is interrupted or permanently discontinued due to toxicities that fulfil the criteria for a DMT, must be followed up at regular intervals (at least once a week), until resolution or stabilization of the event(s), whichever comes first.

Appropriate clinical experts such as cardiologists, endocrinologists, hepatologists, nephrologists etc. should be consulted as deemed necessary.

### Dose Modifying Toxicities (DMTs) for Lutathera

For patients who do not tolerate the protocol-specified dosing schedule, dose adjustments are permitted to allow the patient to continue the study treatment.

The criteria for dose modifications of Lutathera for toxicities are outlined herein.

If a patient requires a dose delay of >16 consecutive weeks of Lutathera from the day of the previous dose, then the patient should be discontinued from the study treatment. In exceptional situations, if the patient is clearly benefiting from the study treatment (i.e. stable disease, partial response, complete response), and in the opinion of the investigator no safety concerns are present, after discussion with the sponsor's Medical Monitor, the patient may remain on the study treatment.

For each patient, a maximum of one dose reduction will be allowed after which the patient will be discontinued from the study.

These dose changes must be recorded on the appropriate electronic case report form (eCRF).

### Dose Modifications of Lutathera for Adverse Events

| | Worst Toxicity CTCAE Grade | |
|---|---|---|
| Adverse Reaction | (unless otherwise specified) | Dose Modification |
| Thrombocytopenia | First occurrence of: | Withhold dose until complete or |
| | Grade 2 (PLT <75 − | partial resolution (Grade 0 to 1). |
| | 50 × 10⁹/L) | If AE resolves before next |
| | Grade 3 (PLT <50 − | planned Lutathera dose (i.e. |
| | 25 × 10⁹/L) | within 9 weeks after the previous |
| | Grade 4 (PLT <25 × 10⁹/L) | dose), administer next full dose |
| | | of Lutathera (7.4 GBq/200 |
| | | mCi). |
| | | If AE resolves within 9-16 |
| | | weeks after the previous dose, |
| | | resume Lutathera at 3.7 GBq |
| | | (100 mCi). If reduced dose does |
| | | not result in Grade 2, 3 or 4 |
| | | thrombocytopenia, administer |
| | | Lutathera at 7.4 GBq (200 mCi) |
| | | for next dose. |
| | | If AE does not resolve within 16 |
| | | weeks after the previous dose, |
| | | permanently discontinue |
| | | Lutathera. |
| | Recurrent Grade 2, 3 or 4 | Permanently discontinue |
| | | Lutathera. |
| Anemia | First occurrence of: | Withhold dose until complete or |
| | Grade 3 (Hgb <8.0 g/dL); | partial resolution (Grade 0, 1, or |
| | transfusion indicated | 2). |
| | Grade 4 (life threatening | If AE resolves before next |
| | consequences) | planned Lutathera dose (i.e. |
| | | within 9 weeks after the previous |
| | | dose), administer next full dose |
| | | of Lutathera (7.4 GBq/200 |
| | | mCi). |
| | | If AE resolves within 9-16 |
| | | weeks after the previous dose, |
| | | resume Lutathera at 3.7 GBq |
| | | (100 mCi). If reduced dose does |
| | | not result in Grade 3 or 4 anemia, |
| | | administer Lutathera at 7.4 GBq |
| | | (200 mCi) for next dose. |
| | | If AE does not resolve within 16 |
| | | weeks after the previous dose, |
| | | permanently discontinue |
| | | Lutathera. |
| | Recurrent Grade 3 or 4 | Permanently discontinue |
| | | Lutathera. |
| Neutropenia | First occurrence of: | Withhold dose until complete or |
| | Grade 3 (ANC <1.0 − 0.5 × | partial resolution (Grade 0, 1, or |
| | 10⁹/L) | 2). |
| | Grade 4 (ANC <0.5 × 10⁹/L) | If AE resolves before next |
| | | planned Lutathera dose (i.e. |
| | | within 9 weeks after the previous |
| | | dose), administer next full dose |
| | | of Lutathera (7.4 GBq/200 |
| | | mCi). |
| | | If AE resolves within 9-16 |
| | | weeks after the previous dose, |
| | | resume Lutathera at 3.7 GBq |
| | | (100 mCi). If reduced dose does |
| | | not result in Grade 3 or 4 |
| | | neutropenia, administer |
| | | Lutathera at 7.4 GBq (200 mCi) |
| | | for next dose. |
| | | If AE does not resolve within 16 |
| | | weeks after the previous dose, |
| | | permanently discontinue |
| | | Lutathera. |
| | Recurrent Grade 3 or 4 | Permanently discontinue |
| | | Lutathera. |
| Renal Toxicity | First occurrence of: | Withhold dose until complete |
| | Creatinine clearance less | resolution. |
| | than 40 mL/min; calculated | If AE resolves before next |
| | using Cockcroft Gault | planned Lutathera dose (i.e. |
| | formula with actual body | within 9 weeks after the previous |
| | weight, or | dose), administer next full dose |
| | 40% increase from baseline | of Lutathera (7.4 GBq/200 |
| | serum creatinine, or | mCi). |
| | 40% decrease from baseline | If AE resolves within 9-16 |
| | creatinine clearance; | weeks after the previous dose, |
| | calculated using Cockcroft | resume Lutathera at 3.7 GBq |
| | Gault formula with actual | (100 mCi). If reduced dose does |
| | body weight. | not result in renal toxicity, |
| | | administer Lutathera at 7.4 GBq |
| | | (200 mCi) as next dose. |
| | | If AE does not resolve within 16 |
| | | weeks after the previous dose, |
| | | permanently discontinue |
| | | Lutathera. |
| | Recurrent renal toxicity Grade | Permanently discontinue |
| | 3 or 4 | Lutathera. |
| Hepatotoxicity | First occurrence of: | Withhold dose until complete or |
| | Bilirubinemia greater than 3 | partial resolution (Grade 0, 1 or |
| | times the upper limit of normal | 2). |
| | (Grade 3 or 4), or | If AE resolves before next |
| | | planned Lutathera dose (i.e. |
| | | within 9 weeks after the previous |
| | | dose), administer next full dose |
| | | of Lutathera (7.4 GBq/200 |
| | | mCi). |
| | | If AE resolves within 9-16 |
| | | weeks after the previous dose, |
| | | resume Lutathera at 3.7 GBq |
| | | (100 mCi). If reduced Lutathera |
| | | dose does not result in G3 or 4 |
| | | bilirubinemia, administer |
| | | Lutathera at 7.4 GBq (200 mCi) |
| | | as next dose. |
| | | If AE does not resolve within 16 |
| | | weeks after the previous dose, |
| | | permanently discontinue |
| | | Lutathera. |
| | Defined as: | Withhold dose until complete or |
| | Hypoalbuminemia less than | partial resolution (Grade 0 to 1). |
| | 30 g/L with International | If AE resolves before next |
| | Normalized Ratio (INR) >1.5. | planned Lutathera dose (i.e. |
| | | within 9 weeks after the |
| | | previous dose), administer next |
| | | full dose of Lutathera (7.4 GBq/ |
| | | 200 mCi). |
| | | If AE resolves within 9-16 |
| | | weeks after the previous dose, |
| | | resume Lutathera at 3.7 GBq |
| | | (100 mCi). If reduced Lutathera |
| | | dose does not result in G2, 3 or |
| | | 4 hypoalbuminemia, administer |
| | | Lutathera at 7.4 GBq (200 mCi) |
| | | as next dose. |
| | | If AE does not resolve within 16 |
| | | weeks after the previous dose, |
| | | permanently discontinue |
| | | Lutathera. |
| | Recurrent hepatotoxicity | Permanently discontinue |
| | | Lutathera. |
| Any other CTCAE* | First occurrence of Grade 3 or | Withhold dose until complete or |
| Grade 3 or Grade 4 | 4 | partial resolution (Grade 0 to 2). |
| AE¹ | | If AE resolves before next |
| | | planned Lutathera dose (i.e. |
| | | within 9 weeks after the |
| | | previous dose), administer next |
| | | full dose of Lutathera (7.4 GBq/ |
| | | 200 mCi). |
| | | If AE resolves within 9-16 |
| | | weeks after the previous dose, |
| | | resume Lutathera at 3.7 GBq |
| | | (100 mCi). If reduced dose |
| | | does not result in Grade 3 or 4 |
| | | toxicity, administer Lutathera at |
| | | 7.4 GBq (200 mCi) as next |
| | | dose. |
| | | If AE does not resolve within 16 |
| | | weeks after the previous dose, |
| | | permanently discontinue |
| | | Lutathera. |
| | Recurrent Grade 3 or 4 | Permanently discontinue |
| | | Lutathera. |
| Severe heart failure | NYHA class III or IV | Permanently discontinue |
| | | Lutathera. |

¹No dose modification required for hematological toxicities Grade 3 or Grade 4 solely due to lymphopenia
*CTCAE: Common Terminology Criteria for Adverse Events, National Cancer Institute

In addition,
permanently discontinue treatment with Lutathera if hypersensitivity due to the active substance or any of excipients of Lutathera is observed;
Temporarily discontinue treatment with Lutathera, until resolution or stabilisation before treatment can be resumed.
   Occurrence of an intercurrent disease (e.g. urinary tract infection);
   Major surgery: wait 12 weeks after the date of surgery, then restart the treatment with Lutathera.

After resolution of a DMT, a patient may receive subsequent planned treatment(s) at the full dose (if resolved within 9 weeks after the previous dose), or at 50% of the standard treatment dose (if resolved within 9-16 weeks after the previous dose), if this is felt to be safe for the patient, or the risk-benefit assessment is favourable. If the same DMT reoccurs after treatment with the reduced Lutathera dose, the patient will remain in the study and continue the scheduled clinical/tumor assessments until tumor progression, but no further Lutathera treatment will be given. Octreotide long-acting 30 mg will be continued at monthly intervals. If the DMT event does not reoccur, the next treatment is at full dose, if it is considered to be safe for the patient, or the risk-benefit assessment is favourable.

If a patient experiences a DMT during Lutathera therapy, subsequent treatments with Lutathera are permissible, provided the DMT resolves within 16 weeks following the non-tolerated administration. In any case, the patient will continue the administration of 30 mg octreotide long-acting.

Lutathera overdose, has a very low probability of occurring since it will be supplied as a single dose “ready to use product” in order to avoid any manipulation outside the production facilities. In addition, the infusion system methods do not allow the concurrent use of two separate Lutathera solution vials (see pharmacy manual). No doubling of the administered radioactivity is ever allowed either in absolute amount or by shortening the time intervals between treatments. Treatments (amount of radioactivity and time of administration) will be monitored during the study and any unallowed treatment modification will be considered a major protocol violation.

### Dose Modifying Toxicities (DMTs) for Octreotide LAR

### Control Arm (Octreotide LAR 60 mg)

In the treatment arm with octreotide long-acting, a dose adjustment will be applied in case of Grade 3 or 4 toxicity, especially in presence of severe abdominal symptoms, and hypoglycaemia/hyperglycemia possibly related to octreotide long-acting. The dose adjustment will also be applied if such adverse events are observed which are unlikely related to the study drug, but to other possible or concomitant causes, and the full administration of octreotide long-acting would represent a safety risk for the patient. If a patient experiences a DMT during octreotide long-acting treatment, the subsequent treatment dose will be reduced from 60 mg to the previous well-tolerated dose (or even temporarily suspended) and then at the next treatment, the dose will be increased to the initial 60 mg dose of octreotide long-acting, if this is felt to be safe for the patient, or the risk-benefit assessment is favorable.

In any case (also in case of octreotide long-acting treatment dose suspension), the patient remains in the study and continues the scheduled clinical/tumor assessments until tumor progression, unless the patient's withdrawal becomes inevitable.

### Lutathera Arm (Octreotide LAR 30 mg)

All DMT recommendations in the control arm (Octreotide LAR 60 mg) are also applicable to the Lutathera arm, where Octreotide LAR is administered at the standard approved dosage (30 mg). Overall, the exact dose reduction levels, duration of suspension and criteria for re-increase and continuation of dose in the control arm will be defined by the treating physician based on clinical judgment and product leaflet.

### Dose Discontinuation

The discontinuation of either study treatments in both arms (Lutathera or octreotide long-acting) is not a reason for patient's withdrawal either from the clinical/tumor assessments until tumor progression, or for early study termination. However, in case of a patient's withdrawal from the clinical/tumor assessments or early study termination (based on either the patient's or the Investigator's decision), patients will undergo all exams scheduled for the End of Treatment (EOT) visit.

If the treatment discontinuation occurs because of laboratory abnormality, or any evidence of toxicity, the Investigator will collect additional specimens for repeat or additional analyses, at intervals appropriate to the abnormality. The patient will be closely followed until sufficient information is obtained to determine the cause or the value regresses. Appropriate remedial measures should be taken and the response recorded in the eCRF.

### Preparation and Dispensation of Lutathera

There is no need for the patients to fast before treatment.

Treatment with Lutathera will consist of a cumulative amount of radioactivity of 29.6 GBq (800 mCi) with the dosing divided among 4 administrations of Lutathera at 8±1-week intervals or up to 16 weeks to accommodate resolving acute toxicity. Each dose is infused over 30 minutes.

In addition to treatment with Lutathera, patients will receive 30 mg octreotide long-acting until the completion of the Treatment Phase.

### Before Each Lutathera Treatment:

1. Unless clinically impossible, octreotide long-acting must not be administered within 6 weeks of the next treatment of Lutathera. In other words, between 2 Lutathera injections performed at regular interval (8 weeks) only one 30 mg octreotide long-acting injection can be administered (possibly one day after Lutathera administration or at least after 4 hours after the end of Lutathera infusion). While not treated with octreotide long-acting, the patient must be treated with an equivalent dosing of short acting octreotide s.c. “Clinically impossible” means that the actual clinical condition of the patient would contraindicate the suspension of treatment because of an otherwise untreatable carcinoid syndrome. After enrollment, octreotide long-acting injections should be planned in order to allow for suitable washout time before first Lutathera treatment. If the time interval between treatments with Lutathera is prolonged for any reason, octreotide long-acting administration continues every 4 weeks, however it should not be administered within 6 weeks of the next Lutathera treatment.
2. Short-acting octreotide is not allowed during the 24 h before the Lutathera treatment date, unless the actual patient clinical conditions would contraindicate the treatment suspension due to otherwise untreatable carcinoid syndrome symptoms. Short-acting octreotide can only be continued if the tumor uptake on SRI imaging during continued somatostatin analogue medication is greater than liver uptake (see Exclusion criteria).
3. Treatment with 30 mg octreotide long-acting can be resumed after the administration of Lutathera. Specifically, the recommended period before resuming octreotide long-acting (or short acting octreotide s.c.) is one day, however the minimum interval to receive octreotide long-acting (or short acting octreotide s.c.) after Lutathera is 4 hours, unless contraindicated as noted above.

The total amount of administered radioactivity is determined by measuring the radioactivity in the Lutathera vial before and after administration.

The scheme for supportive treatment with 30 mg octreotide long-acting is presented in the following table.

Lutathera Arm: schedule for administration of Lutathera, 30 mg octreotide long-acting, and short acting octreotide sc¹.

| Treatment | Wk0 | Wk1 | Wk2 | Wk3 | Wk4 | Wk5 | Wk6 | Wk7 | Wk8 |
|---|---|---|---|---|---|---|---|---|---|
| Lutathera^{2, 3} | X ▪ ▪ ▪ ▪ ▪ ▪ | ▪ | ▪ | ▪ | ▪ | ▪ | ▪ | ▪ | X ▪ ▪ ▪ ▪ ▪ ▪ |
| Octreotide | ▪ Y ▪ ▪ ▪ ▪ ▪ | ▪ | ▪ | ▪ | ▪ | ▪ | ▪ | ▪ | ▪ Y ▪ ▪ ▪ ▪ ▪ |
| long-acting | | | | | | | | | |
| 30 mg⁴ | | | | | | | | | |
| Octreotide | ▪ ▪ ▪ ▪ ▪ ▪ ▪ | ▪ | ▪ | ▪ | Z→ | Z→ | Z→ | ZZZZZZ ▪ | ▪ ▪ ▪ ▪ ▪ ▪ ▪ |
| s.c. short | | | | | | | | | |
| acting | | | | | | | | | |

| | |
|---|---|
| ↑ Octreotide long-acting is resumed the | Octreotide S.C. stopped 24 hr ▪ ↑ |
| day after Lutathera treatment | before Lutathera treatment |

1 Symbols: (X) treatment with Lutathera, (Y) treatment with octreotide long-acting, (Z, Z→) daily injections of octreotide s.c., (▪) no treatment
²Lutathera treatment interval can be increased to 16 weeks to resolve acute toxicity
³If Lutathera treatment is delayed due to DMT, octreotide long-acting may be continued at 4-week intervals, but should not be administered within 6 weeks before next Lutathera treatment; patient should use octreotide s.c. during the washout period
⁴After patients receive all 4 Lutathera administrations at 8 ± 1 week intervals, or Lutathera treatment stopped because of toxicity, patients continue to receive 30 mg octreotide long-acting (1 im injection every 4 weeks) until end of Treatment Phase, unless progression occurs.

On the day of Lutathera treatment, and before the infusion with 2.5% Lys-Arg solution is started, an intravenous bolus of anti-emetic is given (suggested options: Granisetron (3 mg), or Ondansetron (8 mg), or Tropisetron (5 mg)). Prednisone must be avoided as preventive anti-emetic treatment because of potential somatostatin receptor down-regulation. Hyperkaleamia must be corrected prior to 2.5% Lys-Arg infusion if >6.0 mmol/L (CTCAE Grade 3).

In case nausea or vomiting occurs despite this medication, patients can be treated with other anti-emetic drugs at the discretion of the physician.

The sterile amino acid solution and Lutathera are administered in parallel by peripheral vein infusion at a constant infusion rate through pumps or any other infusion system. The infusion with amino acids starts 30 minutes before the start of Lutathera infusion, and continues for a total of 4 h (extension up to 6 h is allowed in case of adverse reactions that require interruption or slowing the infusion rate). During amino acid infusion patient is allowed to void.

Infusion rates are listed in the following table.

### Administration Procedure of Antiemetic, Sterile Amino Acid Solution and Lutathera

| | Start time | Infusion rate | |
|---|---|---|---|
| Administered agents | (min) | (mL/h) | Duration |
| Antiemetic: Granisetron | 0 | as per | as per prescribing |
| 3 mg (or alternative) | | prescribing | information |
| | | information | |
| 2.5% Lys-Arg sterile | 30 | 250 | 4 hours |
| amino acid solution (1 L) | | | |
| Lutathera with sodium | 60 | Up to 400 | 30 ± 10 minutes |
| chloride 9 mg/mL (0.9%) | | | |
| solution for injection | | | |

Following administration of Lutathera, patients should remain at the clinical site for an additional 4 to 5 hours in an area with suitable radiation shielding to protect others from unnecessary exposure. At the time of release, patients are given written instructions which outline the precautions the patient must take to minimize radiation exposure to people around them.

Crises due to excessive release of hormones or bioactive substances may occur following treatment, therefore, observation of patients by overnight hospitalization should be considered. Recommended treatments of patients with hormonal crises are: i.v. high dose somatostatin analogues, i.v. fluids, corticosteroids, and correction of electrolyte disturbances in patients with diarrhea and vomiting.

### Handling of Study Treatment and Additional Treatment

Lutathera, amino acids and octreotide long-acting must be administered at the investigational site. Short-acting octreotide is self-administered by the patient.

The study medication must be stored, handled and administered only by qualified/authorized personnel and must be prepared in accordance with pharmaceutical quality requirements, and radiation safety regulations for Lutathera.

Drug inventory and accountability records for the study medication and rescue medication, as well as drug returns by the patient, will be kept by the Investigator/Pharmacist, and must be documented throughout the study. Returned supplies should not be distributed again, not even to the same patient. The Investigator will not supply investigative study medication to any person, except the patients in this study.

The octreotide long-acting and amino acids not used must be stored at site and made available till the monitoring visits, to allow the CRA to monitor the drug accountability.

The used/unused medications, except for Lutathera, which will be locally discarded according to all disposal requirements for radioactive materials, will be returned to the proper local depot for destruction at the study completion or upon expiration, according to IPM/Sponsor decision and approval.

On an ongoing basis the Investigator/Pharmacist agrees to conduct a study medication supply inventory and to record the results of this inventory on the study Medication Accountability Record. It must be possible to reconcile delivery records with those of used and unused medication. Any discrepancies must be accounted for and explained. Appropriate forms of deliveries and returns must be signed and dated by the responsible person at the clinical site and maintained as records. The return or disposal of all study medication will be documented appropriately.

### Visit Schedule and Assessments

Randomization must be performed once all screening/baseline assessments are complete. The baseline CT/MRI scan should be taken possibly on the same day of randomization or immediately before (within 1 week) to ensure that it reflects the disease status closely before the therapy start. The results of baseline CT/MRI must be assessed by Investigator before randomization.

The screening phase must be shortened as much as possible in order to treat the patients shortly after the informed consent signature.

Randomization should occur as soon as possible after all screening assessments have been completed and eligibility confirmed. As Lutathera production and shipment will take approximately 12 days, if a patient is randomized in the Lutathera arm, Lutathera first dose must be ordered immediately after randomization.

During the study, patients will be evaluated for safety and tolerability in accordance with the Visit Schedules for the Lutathera arm and the octreotide long-acting arm (variations of ±1 week in the visits schedule are allowed).

Subjects should be seen for all visits/assessments as outlined in the assessment schedule or as close to the designated day/time as possible. Missed or rescheduled visits should not lead to automatic discontinuation. Subjects who prematurely discontinue the study treatment for any reason should be scheduled for a visit as soon as possible, at which time all of the assessments listed for the EOT/Early Termination visit will be performed. At the EOT/Early Termination visit, all dispensed investigational product should be reconciled, and the adverse event and concomitant medications recorded on the eCRF.

As the date of randomization and the date of first treatment might not be the same, it should be observed that a misalignment may occur between the tumor imaging exams (CT/MRI scans) which are scheduled from the date of randomization, and the other clinical and laboratory assessments which are scheduled from the first treatment date.

The assessments listed in the following table will be performed centrally. Procedures for centralized evaluations will be detailed in the Laboratory Manuals provided to each participating site.

Assessment schedule lists all of the assessments and when they are performed. All data obtained from these assessments must be supported in the subject's source documentation.

### Centrally Performed Assessments.

| | | Study |
|---|---|---|
| Assessment | Material to be Delivered | Phase |
| SRI tumor uptake in all documented | SRI modality: [68Ga]-DOTA- | Screening |
| target lesions will be assessed locally at | TOC (e.g. Somakit-TOC ®) | & before |
| screening to verify that tumor uptake in | PET/CT (or MRI when | cross- |
| all target lesions is greater than the liver | applicable based on target | over/re- |
| uptake. | lesions) imaging or [68Ga]- | treatment |
| If the patient will participate to the | DOTA-TATE PET/CT (or MRI | |
| optional Treatment Extension Phase | when applicable based on | |
| after progression, SRI tumor uptake in | target lesions) imaging (e.g. | |
| all documented target lesions will be re- | NETSPOT ®) Somatostatin | |
| assessed locally before cross-over. | Receptor scintigraphy (SRS) | |
| Images will be also submitted to the | with [111In]-pentetreotide | |
| central imaging center for a second | (Octreoscan ® SPECT/CT), | |
| review (there is no central ‘real time’ | SRS with [99mTc]-Tektrotyd, | |
| assessment of the SRI images, | [64Cu]-DOTA-TATE PET/CT | |
| however the images should be | (or MRI when applicable | |
| submitted to the central imaging center | based on target lesions) | |
| possibly within 1 month. SRI images | imaging. | |
| submission is not required for screening | SRI obtained within 3 months | |
| failure patients. | of randomization will be | |
| | accepted for inclusion; if older, | |
| | the exam will need to be | |
| | repeated at screening. | |
| | If the patient participates to the | |
| | optional Treatment Extension | |
| | or re-treatment Phase (after | |
| | progression), SRI imaging | |
| | must be repeated before cross- | |
| | over/re-treatment. The SRI | |
| | images should be submitted to | |
| | the central imaging center | |
| | (uploading into the dedicated e- | |
| | platform) possibly within 1 | |
| | month. | |
| | See Imaging Manual for further | |
| | details. | |
| Objective tumor response according to | CT/MRI scans. | Treatment |
| RECIST Criteria. | | Phase |
| Tumor response according to RECIST | It is recommended that for each | |
| is re-evaluated centrally (real-time | patient identical acquisition and | |
| reading) during the study. | reconstruction protocols be | |
| | used at all time-points. | |
| | The images must be submitted | |
| | (uploading into the dedicated e- | |
| | platform) to the central imaging | |
| | center as soon as possible for | |
| | real-time assessment. | |
| | See Imaging Manual for further | |
| | details. | |
| Measurements of the RR, PR, QRS, | 12-lead ECGs will be | Baseline & |
| and QT interval durations. | performed using the device | Treatment |
| 12-lead ECGs in triplicate will be re- | provided by Sponsor. | Phase |
| assessed centrally at baseline and | Data will be transferred after | |
| during the study. | acquisition to the central | |
| | laboratory for the central | |
| | cardiologist assessment. | |
| | See ECG Manual for further | |
| | details. | |
| PK of high dose octreotide long-acting | Blood will be collected using | Treatment |
| (60 mg), to evaluate steady-state trough | the sampling tubes provided by | Phase/ |
| plasma concentration assessment (all | Sponsor. The storage | control |
| Countries except China). | temperature for PK samples | arm |
| Blood samples (2.5 mL each sample) | should be at or below minus | only |
| will be collected at the pre-specified pre- | 20° C. (−20° C.). | |
| dose time-points to yield approximately | Samples will be shipped | |
| 1 mL plasma for analysis of octreotide | regularly to the central | |
| concentration. | laboratory for central PK | |
| | assessment. | |
| | See PK Manual for further | |
| | details. | |

### Demographics and Baseline Characteristics

Each patient's date of birth, gender, ethnicity, weight, height and relevant baseline characteristics will be recorded in the e-CRF.

### Diagnosis and Extent of Cancer

The patient's disease history, including documented primary diagnosis of gastro-entero-pancreatic tumor, date of diagnosis (diagnosis date of metastasized or locally advanced disease should not be greater than 6 months prior to screening), as well as disease status at study entry, will be collected. This includes the date of first diagnosis and presence of metastases with specification of the metastatic site(s). TNM criteria will be used for the determination of the stage of disease at the time of first diagnosis.

### Ki67

All patients are required to have documented local assessment of the Ki67 proliferation index based on surgery/biopsy specimens of the primary tumor or liver metastases or soft tissue metastases and assessed by microscopy and immunohistochemical staining.

Ki67 must be ≥10% and ≤55% for a patient to be eligible.

### Prior Antineoplastic Medications/Radiotherapy/Surgery

Information pertaining to any chemotherapy, hormonal therapy, immunotherapy, radiation, or surgery the patient has previously received will be documented. Previous treatment with somatostatin analogs will be also documented if applicable.

### SRI Tumor Uptake in Documented Lesions

All patients are required to have [68Ga]-DOTA-TOC (e.g. Somakit-TOC®) PET/CT (or MRI when applicable based on target lesions) imaging or [68Ga]-DOTA-TATE PET/CT (or MRI when applicable based on target lesions) imaging (e.g. NETSPOT®) or Somatostatin Receptor scintigraphy (SRS) with [111In]-pentetreotide (Octreoscan® SPECT/CT) or SRS with [99mTc]-Tektrotyd or [64Cu]-DOTA-TATE PET/CT (or MRI when applicable based on target lesions) imaging performed within 3 months prior to the projected randomization date in the study (available exams older than 3 months need to be repeated to evaluate the patient's eligibility).

SRI imaging must be repeated before cross-over (after progression in the control arm), if the patient participates to the optional Treatment Extension Phase, and before the optional re-treatment (after progression in the Lutathera arm).

SRI tumor uptake in documented target lesions will be assessed locally to evaluate patient's eligibility before randomization, cross-over and re-treatment. Tumor uptake in all target lesions must be greater than the liver uptake.

Images will be also submitted to the central imaging center for a second review: there is no central ‘real time’ assessment of the SRI images, however the images should be submitted to the central imaging center possibly within 1 month. SRI images submission is not required for screening failure patients.

Lytic bone lesions, with an identifiable soft tissue component, evaluated by CT or MRI, can be considered as measurable lesions if the soft tissue component otherwise meets the definition of measurability according to RECIST 1.1 (in any case, blastic bone lesions are not measurable). Therefore, if the bone lesion has the characteristic as above, and SRI is positive, the patient may be enrolled.

If the primary tumor has been resected, tumor evaluations including SRI will be performed on metastases.

Operating details and quality certificates are specified in the Imaging Manual and Imaging Charter.

### Prior/Concomitant Medications

All medications taken at the start of screening until the end of the Treatment Phase/optional Treatment Extension Phase/optional Re-treatment Phase, or early termination, are to be recorded. This includes prescription and over-the-counter medications taken during this time frame.

During the Follow up Phase, concomitant medications must be collected only if administered for related SAEs/AESI and/or for secondary hematological malignancies. In addition, further anti-tumor treatments administered after progression must be reported until the end of the Follow up Phase.

If any additional medication or procedure has been given for treatment of suspected or confirmed COVID-19 during the study, the site is requested to please enter them as a concomitant medication or procedure (e.g. intubation) and state/select ‘reason’ to match the relevant Adverse Event reported when applicable.

Tests performed to diagnose symptoms of COVID-19 should be reported as Adverse Events only and not duplicated in Concomitant Medications/Surgical and Medical Procedures or equivalent CRFs.

### Efficacy Assessment

### 1. Progression Free Survival

The primary efficacy end-point is PFS as measured by objective tumor response, which is determined by RECIST criteria, Version 1.1 (Eisenhauer, et al., 2009). Tumor response will be assessed locally and centrally.

Triphasic CT imaging is the preferred modality over MRI for determining objective tumor response. Combined PET/CT may be used only if the CT is of similar diagnostic quality as a CT performed without PET, including the utilization of IV contrast media.

The baseline CT/MRI scan should be taken possibly on the same day of randomization or immediately before (within 1 week) to ensure that it reflects the disease status closely before the therapy start. The results of baseline CT/MRI must be assessed by the Investigator before randomization.

The tumor diameters of indicator lesions used for response assessment should be measured in the closest position to that used for the baseline CT or MRI assessment.

Restaging is scheduled at regular intervals in the two treatment arms (week 16±1, week 24±1 and then every 12±1 weeks) starting from the date of randomization. Every effort should be done to avoid differences between these timings for patients in the two arms. In case of delays, the reason of the delay has to be documented and the CT/MRI assessment has to be done as soon as possible.

If a CT/MRI scan is done on the same day of a Lutathera administration (e.g. in case of therapy postponement due to DMT or scheduling issues), the scan should be taken before the drug administration. When a CT/MRI assessment is >6 weeks earlier or later than the original schedule, the next reassessments have to be discussed and adjusted with the Medical Monitor of the study in a way to progressively come back to the original schedule.

It is recommended that for each patient identical acquisition and reconstruction protocols be used at all time-points. Central, blinded, real-time IRC (Independent Review Committee) assessment will be conducted for determining progressive disease. Changes from randomization date will be assessed at week 16±1, week 24±1 and then every 12±1 weeks until the PFS primary analysis End-Point has been reached, then until Week 72 after randomization, unless the patient progresses or dies.

Sponsor will notify all the Centers and their Ethic Committees as soon as the 99 PFS events have occurred.

Additional PFS/PFS2 data will be collected up to 3 years from the end of the Treatment Phase of the last patient (during the follow-up phase RECIST 1.1 assessment will be performed only locally every 6 months (±1 month)).

Additional imaging assessments may be performed at any time during the study at the investigator's discretion to support the efficacy evaluations for a subject, as necessary. Clinical suspicion of disease progression at any time requires a physical examination and imaging assessments to be performed promptly rather than waiting for the next scheduled imaging assessment. These additional scans should be provided to the IRC for central assessment. If an off-schedule imaging assessment is performed because progression is suspected, subsequent imaging assessments should be performed in accordance with the original imaging schedule.

After progression and during the follow-up every effort must be made by the Investigator to collect additional information on further anti-tumor therapies and scan assessments outcome (RECIST 1.1 local evaluation) to evaluate the Time to Second Progression (PFS2) in the two study arms.

For progressive patients participating in cross-over or re-treatment, CT/MRI scans performed after the cross-over or re-treatment eligibility will follow the visits schedule counted from the 1st Lutathera dose.

For patients that are re-treated with Lutathera, a separate PFS analysis will be conducted during the re-treatment phase using the same definition except that the baseline will be based on the tumor assessment which demonstrated progression thus allowing re-treatment with Lutathera. This PFS analysis during re-treatment will be descriptive only.

Operating details and quality certificates are specified in the Imaging Manual and Imaging Charter.

### Objective Response Rate (ORR), Duration of Response (DOR), Time to Second Progression (PFS2)

Objective Response Rate (ORR) will be calculated as the rate of patients with a best overall response of partial response (PR) or complete response (CR). Response duration will be calculated from the time of initial response until documented tumor progression. The Duration of Response (DOR) is defined as the time from initially meeting the criteria for response (CR or PR) until the time of progression by RECIST or death due to underlying disease. DOR will be reported descriptively for each group without comparison between groups.

As additional secondary exploratory end-point (local RECIST assessment), the Time to Second Progression (PFS2) will be assessed in the two study arms. PFS2 is defined as the time from randomization to the time of disease progression or death due to any cause (on any treatment) following the first episode of disease progression.

For patients that are re-treated with Lutathera, a separate analysis for ORR, DoR, and PFS2 will be conducted during the re-treatment phase using the same definition as for ORR and DoR, except that the new baseline will be based on the tumor assessment which demonstrated progression thus allowing re-treatment with Lutathera. The PFS2 definition is the same. This ORR, DoR, and PFS2 analysis during re-treatment will be descriptive only. It is recognized that the limitation of including cross-over and the lower scans frequency during follow-up will reduce the possibility to reach firm conclusions on PFS2.

After the first progression is confirmed centrally and the second progression is confirmed locally (i.e. after PFS2 event), the sites which do not routinely perform 6-monthly follow up CT/MRI scans for progressive GEP-NET patents can omit this examination at the subsequent patient's visits (no protocol deviation).

### Overall Survival

Overall Survival (OS) will be calculated from the randomization date until the day of death due to any cause; OS will not be censored if a patient receives other anti-tumor treatments after study medication.

Overall Survival will also be separately estimated for patients that receive re-treatment with Lutathera. The definition for OS is the same, and this analysis with re-treatment patients will be descriptive only.

Survival data will be analyzed at the time of the analysis of the primary end-point (PFS), and will continue to be assessed up to 4 years from the randomization of the last patient or 6 months after the last cross-over/re-treatment dose in the study, whichever occurs last.

### Quality of Life

The impact of treatment on health related QoL will be assessed using the EORTC QLQ-G.I.NET21, EORTC QLQ-C30 and EQ-5D-L5 questionnaires, which will be filled in by the patient prior to know CT scan/MRI result. Changes from baseline will be assessed every 12±1 week from the first treatment date until the end of treatment.

Forms in Country-specific languages will be provided by the Sponsor.

### Safety and Tolerability

### Adverse Events

All adverse events (AEs), whether or not spontaneously reported by the patient, will be recorded starting from the signing of the ICF until the end of the Treatment Phase/optional Treatment Extension Phase.

During the Follow up Phase only related SAEs and related AESI other than secondary hematological malignancies will be recorded. AESI of secondary hematological malignancies will be recorded during the whole study irrespective of causality. An Independent Steering Committee Board will evaluate patient's safety throughout the study. During the Follow-up Phase, concomitant medications must be collected in the eCRF if administered for related SAEs/AESI and/or for secondary hematological malignancies.

### Laboratory Assessments

The laboratory assessments require that blood samples for hematology and blood chemistry, and a urine sample for urinalysis are taken. Laboratory assessments will be performed at the investigational site.

At Screening: all patients will have screening laboratory assessments including hematology and blood chemistry within 3 weeks (preferably 2 weeks in the Lutathera arm) before the projected first treatment date.

### During the Treatment Phase (Including Cross-Over and Re-Treatment):

In the Lutathera arm: within 2 weeks before and 4±1 weeks after each Lutathera treatment. In addition, for the second, third, and fourth Lutathera treatment, an additional laboratory assessment will be performed on the same day, or within one day prior Lutathera administration. Blood tests performed 4 weeks after any treatment cannot serve as baseline values for the next treatment. After the end of Lutathera treatment, laboratory assessments will be performed every 12±1 weeks.
In the 60 mg octreotide long-acting arm: throughout the study laboratory assessments will be performed 4 weeks (±3 days) after the first treatment till Week 28, and every 12±1 weeks from Week 36 onwards.

In the event of a significant laboratory abnormality, or if clinical or laboratory evidence of toxicity occurs, the Investigator will collect additional specimens for repeat or additional analyses, at intervals appropriate to the abnormality. The patient will be closely followed until sufficient information is obtained to determine the cause or the value regresses. Appropriate remedial measures should be taken and the response recorded.

All safety laboratory results must be evaluated by the Investigator before administration of study medication.

Any clinically relevant change from baseline onwards will be recorded on the Adverse Event page of the e-CRF, possibly with a single diagnosis encompassing all changes possibly supporting to the single diagnosis.

During the COVID-19 pandemic, the investigator should consider whether it is possible to arrange for the laboratory tests to be performed at a local laboratory for those patients who cannot come to the clinical site as scheduled. The investigator should collect and file all local lab reports in the patient medical record or chart.

### Laboratory Assessments¹.

| Haematology | Blood Chemistry | Pregnancy test |
|---|---|---|
| WBC with differential² | BUN or urea | Pregnancy test, if |
| Platelets² | Serum creatinine³ | applicable (urine |
| Hb² | Creatinine Clearance³ | pregnancy test is |
| MCV | Uric acid | accepted⁵) |
| Haematocrit | Albumin | |
| | Total bilirubin³ | |
| | AP | |
| | AST/ASAT | |
| | ALT/ALAT | |
| | Gamma-GT | |
| | Sodium | |
| | Potassium | |
| | Magnesium | |
| | LDH | |
| | CgA⁴ | |
| | GlycoHb (haemoglobin | |
| | A1C) | |
| | TSH (and free Thyroxine | |
| | (fT4) if TSH is abnormal) | |
| | Calcium | |
| | Fasting blood Glucose | |

¹Laboratory assessments performed on the same day or within one day prior to administration of the second, third, and fourth doses of Lutathera must include at minimum: serum urea or blood urea nitrogen and creatinine (including creatinine clearance calculation) serum potassium, serum total bilirubin, aspartate aminotransferase and alanine aminotransferase hemoglobin, platelet count, total leukocyte count, and absolute neutrophil count.
²Before each Lutathera dose, patients must meet the criteria for Hb, WBC, and platelets, as defined in the inclusion criteria, at baseline and before each subsequent treatment. If the patient cannot be retreated due to haematological abnormalities, the evaluation must be repeated at least once weekly until re-treatment.
³Before each Lutathera dose, patients must meet the criteria for creatinine clearance and total bilirubin as defined in the eligibility criteria at baseline and before every re-treatment. As entry criterion, patients must not have creatinine clearance <40 mL/min calculated by the Cockroft Gault method. During the course of the study, if in the Lutathera arm a 40% increase over the baseline serum creatinine value occurs during the course of treatment, or a decrease of over 40% in creatinine clearance as calculated from serum creatinine concentrations according to Cockcroft-Gault formula, DMT rules must apply and patients must also have a measured creatinine clearance (or GFR) performed. Measured creatinine clearance should be through a 24-h urine collection. Total urinary protein should also be measured in this collection. If the measured urinary creatinine clearance shows decrease of ≤40% compared to baseline, treatment can continue. The Cockcroft-Gault formula allows this estimation based on the occurrence of creatininemia, and correlating patient
muscular mass (and so the consequent creatinine production) to weight, gender and age: Est. Creatinine Clearance = [[140 - age (yr)]*weight (kg)]/[72*serum Cr(mg/dL)] (multiply by 0.85 for women) Or Est. Creatinine Clearance = [[140-age(yr)]*weight (kg)]/[0.814* serum Cr(umol/L)] (multiply by 0.85 for women)
⁴In South Korea only, CgA assessment being discontinued in Q3 2022 due to lab kits shortage
⁵For UK, serum pregnancy test is mandatory at baseline

During the Follow-up Phase, in both study arms laboratory assessments (hematology and blood chemistry) will be performed every 6 months (±1 month).

### Safety Monitoring and Reporting

### Definition of Adverse Events and Reporting Requirements

### Adverse Events

An adverse event (AE) is any untoward medical occurrence (e.g., any unfavorable and unintended sign [including abnormal laboratory findings], symptom or disease) in a subject or clinical investigation subject after providing written informed consent for participation in the study. Therefore, an AE may or may not be temporally or causally associated with the use of a medicinal (investigational) product.

The investigator has the responsibility for managing the safety of individual subject and identifying adverse events.

Abnormal laboratory values or test results occurring after informed consent constitute adverse events only if they induce clinical signs or symptoms, are considered clinically significant, require therapy (e.g., hematologic abnormality that requires transfusion or hematological stem cell support), or require changes in study medication(s).

The investigator has the responsibility for managing the safety of individual subject and identifying adverse events.

Adverse events that begin or worsen after informed consent must be recorded in the Adverse Events eCRF. Conditions that were already present at the time of informed consent must be recorded in the Medical History page of the subject's eCRF. Adverse event monitoring must be continued for at least 30 days (or 5 half-lives, whichever is longer) following the last dose of study treatment. Adverse events (including lab abnormalities that constitute AEs) must be described using a diagnosis whenever possible, rather than individual underlying signs and symptoms. When a clear diagnosis cannot be identified, each sign or symptom must be reported as a separate Adverse Event.

The occurrence of adverse events must be sought by non-directive questioning of the subject at each visit during the study. Adverse events also may be detected when they are volunteered by the subject during or between visits or through physical examination findings, laboratory test findings, or other assessments.

Adverse events must be recorded under the signs, symptoms, or diagnosis associated with them, accompanied by the following information (as far as possible):
1. The Severity grade OR the Common Toxicity Criteria (CTC) AE grade.
   a. If “severity grade” is selected, include the following:
      i. mild: usually transient in nature and generally not interfering with normal activities
      ii. moderate: sufficiently discomforting to interfere with normal activities
      iii. severe: prevents normal activities
   b. If “CTCAE grade” is selected, include the following:
      i. Adverse events will be assessed and graded from 1 to 5.
2. Its relationship to the study treatment. If the event is due to lack of efficacy or progression of underlying illness (i.e. progression of the study indication) the assessment of causality will usually be ‘Not suspected.’ The rationale for this guidance is that the symptoms of a lack of efficacy or progression of underlying illness are not caused by the trial drug, they happen in spite of its administration and/or both lack of efficacy and progression of underlying disease can only be evaluated meaningfully by an analysis of cohorts, not on a single patient
3. Its duration (start and end dates) or if the event is ongoing, an outcome of not recovered/not resolved must be reported.
4. Whether it constitutes a SAE and which seriousness criteria have been met.
5. Action taken regarding with study treatment
6. All adverse events must be treated appropriately. Treatment may include one or more of the following:
   Dose not changed
   Dose Reduced
   Drug interrupted/withdrawn
7. Its outcome i.e., its recovery status or whether it was fatal

If the event worsens the event should be reported a second time in the eCRF noting the start date when the event worsens in toxicity. For grade 3 and 4 adverse events only, if improvement to a lower grade is determined a new entry for this event should be reported in the eCRF noting the start date when the event improved from having been Grade 3 or Grade 4.

Conditions that were already present at the time of informed consent should be recorded in the medical history of the patient.

Adverse events (including lab abnormalities that constitute AEs) should be described using a diagnosis whenever possible, rather than individual underlying signs and symptoms. Once an adverse event is detected, it must be followed until its resolution or until it is judged to be permanent (e.g. continuing at the end of the study), and assessment must be made at each visit (or more frequently, if necessary) of any changes in severity, the suspected relationship to the interventions required to treat it, and the outcome.

Information about adverse drug reactions for the investigational drug can be found in the Investigator's Brochure (IB).

Abnormal laboratory values or test results constitute adverse events only if they fulfill at least one of the following criteria:
they induce clinical signs or symptoms.
they are considered clinically significant.
they require therapy

Clinically significant abnormal laboratory values or test results must be identified through a review of values outside of normal ranges/clinically notable ranges, significant changes from baseline or the previous visit, or values which are considered to be non-typical in subjects with the underlying disease.

Progression of GEP-NET (including fatal outcomes), if documented by use of appropriate method, should not be reported as a serious adverse event, except if the investigator considers that progression of malignancy is related to study treatment.

If patients in the study have been infected and/or tested for SARS-COV-2 (COVID-19), sites should capture the patient-relevant details in the patient charts/source data, i.e. start and end dates of suspected infection when information is available, any date of test(s) performed, test name/method together with the result (positive/negative), treatment interruptions or discontinuations for each of the impacted patients enrolled in the trial.

If an infection is suspected based on symptoms suggestive of COVID-19, sites are requested to enter an AE with AE term as ‘Suspected COVID-19’ or as ‘Confirmed COVID-19’ if confirmed by testing (e.g. antigen/radiologic).
For Suspected cases, if a test is performed and confirms COVID-19, sites are requested to update the Suspected COVID-19 AE record with AE term as ‘Confirmed COVID-19’, however, not altering the start date.
For Suspected cases, if a test is performed and does not confirm COVID-19, sites are requested to follow the normal Adverse Event reporting process and record the AE term as the diagnosis, or if no diagnosis, the symptoms. If COVID-19 is still suspected (e.g. false negative suspected), the AE term can remain as ‘Suspected COVID-19’ per the Investigator's judgement.

In normal circumstances, “testing” for COVID-19 would not be reported as an Adverse Event, however, in order to adequately capture the impact of COVID-19 on trial patients, these will be required to understand the patients' health status and sequence of events for tests performed to diagnose symptoms of COVID-19.
If a test was/tests were performed; sites are requested to enter additional AE(s) with appropriate AE term for the type of test with the result, such as ‘SARS-COV-2 test negative’, ‘SARS-COV-2 test positive’, etc.
Start Date and End Date should be entered as the date of the test
All other fields on Adverse Event CRFs should remain blank. Any resulting queries will be closed by Data Management
Tests performed after a patient has died should not be reported as Adverse Events

The infection is considered serious and to be reported as Serious Adverse Event (SAE) only when it fulfills the protocol definition of a Serious Adverse Events.

### Serious Adverse Events

An SAE is defined as any adverse event [appearance of (or worsening of any pre-existing)] undesirable sign(s), symptom(s) or medical conditions(s)) which meets any one of the following criteria:
fatal
life-threatening.
Life-threatening in the context of a SAE refers to a reaction in which the subject was at risk of death at the time of the reaction; it does not refer to a reaction that hypothetically might have caused death if it were more severe (please refer to the ICH-E2D Guidelines).
results in persistent or significant disability/incapacity
constitutes a congenital anomaly/birth defect
requires inpatient hospitalization or prolongation of existing hospitalization, unless hospitalization is for:
   Routine treatment or monitoring of the studied indication not associated with any deterioration in condition.
   Elective or pre-planned treatment for a pre-existing condition that is unrelated to the indication under study and has not worsened since signing the information consent
   social reasons and respite care in the absence of any deterioration in the subject's general condition
   treatment on an emergency outpatient basis for an event not fulfilling any of the definitions of a SAE given above and not resulting in hospital admission
   medically significant, e.g. defined as an event that jeopardizes the subject or may require medical or surgical intervention to prevent one of the outcomes listed above.

Medical and scientific judgment should be exercised in deciding whether other situations should be considered serious reactions, such as important medical events that might not be immediately life threatening or result in death or hospitalization but might jeopardize the subject or might require intervention to prevent one of the other outcomes listed above. Such events should be considered as “medically significant”. Examples of such events are intensive treatment in an emergency room or at home for allergic bronchospasm, blood dyscrasias or convulsions that do not result in hospitalization or development of dependency or abuse (please refer to the ICH-E2D Guidelines).

All malignant neoplasms will be assessed as serious under “medically significant” if other seriousness criteria are not met and the malignant neoplasm is not a disease progression of the study indication.

Any suspected transmission via a medicinal product of an infectious agent is also considered a serious adverse reaction.

All reports of intentional misuse and abuse of the product are also considered serious adverse event irrespective if a clinical event has occurred.

COVID-19 infections are considered serious and have to be reported as Serious Adverse Event (SAE) only when it fulfills the protocol definition of a Serious Adverse Events. All SAE data should be entered as per the study level CRF completion guidelines e.g. report outcome as ‘fatal’ if the patient died due to COVID-19.

In case a patient died due to COVID-19, it must be clear from the eCRF if the death was due to underlying COVID-19 or a different reason. Therefore, ‘Primary Cause/Reason for Death’ should indicate ‘Suspected COVID-19’ or ‘Confirmed COVID-19’. If a test is performed after the patient died and the assessment indicates death was due to COVID-19 infection, the ‘Primary Cause/Reason for Death’ should be retrospectively changed from ‘Suspected COVID-19’ to ‘Confirmed COVID-19’.

### Adverse Drug Reaction (ADR)

An ADR is any noxious and unintended response to an IMP related to any dose with at least a reasonably possible causal relationship with the IMP. Briefly, an ADR is an AE which is suspected to be possibly related to IMP by either the investigator or the study sponsor.

### Suspected Unexpected Serious Adverse Reactions (SUSARs)

An ADR will be assessed to be “unexpected” if the nature, severity or frequency of the event is not consistent with the applicable product information available for the IMP. An ADR will be assessed to be “expected” if it is listed in the Investigator's Brochure.

A SUSAR is an adverse event regarded as serious with at least possible causal relationship to the drug, the nature, severity or frequency of which is not consistent with the applicable information available in the reference documents available for the IMP.

In the case of octreotide, “expectedness” will be assessed based on octreotide Reference Safety Information (RSI) reported in the prescribing information.

### Adverse Events of Special Interest

AESI are defined as events (serious or non-serious) which are ones of scientific and medical concern specific to the Sponsor's product or program, for which ongoing monitoring and rapid communication by the Investigator to the Sponsor is appropriate. Such events require further investigation in order to characterize and understand them.

AESI are defined based on the NETTER-1 study established AESI list as well as on the basis of an ongoing review of the safety data collected during Lutathera clinical programs and post marketing.

These potential risks deserve special attention even if they do not fulfill any of the seriousness criteria. AESI occurring in patients enrolled in both arms should be reported to the Sponsor for safety analysis following the reporting procedure and timelines for SAEs. During the treatment phase, all AESI need to be reported to the Sponsor irrespective of causality. During the follow-up phase, AESI need to be reported only if considered related to the study treatment except for all secondary hematological malignancies which need to be reported as AESI irrespective of causality.

The following AESI categories representing main risks of Lutathera and amino acid treatment are listed in the following table.

### Adverse Events of Special Interest

AESI categories
Hematotoxicity
Secondary hematological malignancies
Nephrotoxicityg
Cardiovascular and electrolyte disorders

Hematotoxicity: The main critical organ of Lutathera treatment is the bone marrow. Significant hematotoxicity, defined as Grade 2 or higher thrombocytopenia, or Grade 3 or 4 of anaemia, leuko-/neutropenia are considered dose-modifying toxicities in the study and must be reported as AESI when not strictly fulfilling the criteria of serious adverse events. Haematological toxicities regardless of severity must be reported as AESI if accompanied by clinical consequences, i.e., infections in the presence of leuko-/neutro-/lymphopenia, hemorrhages/purpuric lesions under thrombocytopenia that is not explained by another coagulation disorder, dyspnea/fatigue in the presence of anaemia not otherwise explained by the underlying carcinoid syndrome or other co-morbidity.

Secondary haematological malignancies: any secondary hematological malignancies, irrespective of causality, including MDS and acute myeloid leukemia, should be reported in every case, as AESI/SAE.

Nephrotoxicity: Since Lutathera is cleared through the kidneys and reabsorbed by the kidneys, the kidneys have always been considered the “critical organs”. An infusion of sterile amino acid is used for kidneys protection by inhibition of tubular reabsorption of Lutathera. Pursuant to these risk minimization efforts and in addition to the criteria of dose-modifying toxicities and criteria of inclusion (at baseline and before subsequent treatments) pertinent to renal function measurements, renal and urinary tract toxicities are considered AESI. Investigators must report as AESI renal toxicities, including renal failure (ranging from significantly reduced measured or estimated creatinine clearance to clinically overt renal failure other than that of obvious non-IMP-induced origin), suspected radiation nephropathy of any type, such as radiation-induced thrombotic microangiopathy (manifested with, e.g., proteinuria, hypertension, edema, anaemia, decrease serum haptoglobin), or general symptoms and signs of acute radiation toxicity (e.g., increased frequency and urgency of urination, nocturia, dysuria, bladder spasm, bladder obstruction, genitourinary ulceration or necrosis).

Cardiovascular and electrolyte disorders: All clinically significant changes in blood pressure, heart rate, and electrocardiogram parameters must be reported as AESI if they occur within reasonable propinquity of Lutathera and/or amino acid administration in the judgment of the Investigator. Likewise, clinically manifest and/or consequences of hypo-/hypertension, arrhythmias, cardiac conduction disturbances, and other cardiac pathologies evidenced by objective findings/changes on electrocardiogram or echocardiography should also be considered for AESI reporting. In addition, any clinically significant event of hyperkalemia or hypokalemia must be reported as AESI.

### Data Analysis and Statistical Methods

The primary efficacy and safety analyses will be performed after observing approximately 99 PFS events per central assessment. The primary CSR will be produced after the primary PFS analysis. The key secondary endpoints will be tested hierarchically at the time of the primary PFS analysis. Any additional data for participants continuing to receive study treatment past this time and for participants continuing for efficacy follow-up (PFS, OS) and quality of life follow-up, as allowed by the protocol, will be further summarized at the time of the final analysis. A final report to support the final analysis will be planned after End of Study (EOS).

Advanced Accelerator Applications and/or a designated contract research organization (CRO) will perform all analysis.

Any data analysis carried out independently by the investigator should be submitted to Advanced Accelerator Applications before publication or presentation.

### Analysis Sets

The Full Analysis Set (FAS) comprises all subjects to whom study treatment has been assigned by randomization. According to the intent to treat principle, subjects will be analyzed according to the treatment and strata they have been assigned to during the randomization procedure.

The Safety Set includes all subjects who received at least one dose of study treatment. Patients will be analyzed according to the study treatment received, where treatment received is defined as the randomized treatment if the subject took at least one dose of that treatment or the first treatment received if the randomized/assigned treatment was never received.

The pharmacokinetic analysis set (PAS) consists of all randomized patients who received at least one dose of study drug and had at least one post dosing evaluable PK assessment. The cross-over set is comprised of all patients randomized to the Sandostatin LAR Depot (octreotide long-acting) arm who received at least one dose of Lutathera after cross-over following confirmed disease progression per central, blinded, real-time images reading in the randomized period.

The re-treatment set is comprised of all patients randomized to the Lutathera arm who received at least one dose of Lutathera during re-treatment period after confirmed disease progression per central, blinded, real-time images reading in the randomized period.

### Subject Demographics and Other Baseline Characteristics

Demographic and other baseline data including disease characteristics will be listed and summarized descriptively by treatment group for the FAS and Safety set.

Categorical data will be presented as frequencies and percentages. For continuous data, mean, standard deviation, median, minimum, and maximum will be presented. For selected parameters, 25th and 75th percentiles will also be presented.

Relevant medical history and current medical conditions at baseline will be summarized separately by system organ class and preferred term, by treatment group.

### Treatments

The Safety set will be used for the analyses below. Categorical data will be summarized as frequencies and percentages. For continuous data, mean, standard deviation, median, 25th and 75th percentiles, minimum, and maximum will be presented.

The duration of exposure in months to Lutathera plus Standard Dose octreotide long-acting (30 mg), and, High Dose octreotide long-acting (60 mg) as well as the dose intensity (computed as the ratio of actual cumulative dose received and actual duration of exposure) and the relative dose intensity (computed as the ratio of dose intensity and planned dose intensity) will be summarized by means of descriptive statistics using the safety set.

Concomitant medications and significant non-drug therapies prior to and after the start of the study treatment will be listed and summarized according to the Anatomical Therapeutic Chemical (ATC) classification system, by treatment group.

The number of subjects with dose adjustments (reductions, interruption, or permanent discontinuation) and the reasons will be summarized by treatment group and all dosing data will be listed.

### Analysis of the Primary Endpoint(s)

The primary aim of the study is to demonstrate a prolongation of Progression Free Survival (PFS) time (centrally assessed according to RECIST 1.1).

### Definition of Primary Endpoint(s)

The primary endpoint of the study is progression-free survival (PFS), defined as the time from the date of randomization to the date of the first documented progression or death due to any cause. PFS will be assessed via central review according to RECIST 1.1.

### Statistical Model, Hypothesis, and Method of Analysis

Assuming proportional hazards model for PFS, the null hypothesis will be tested at one-sided 2.5% level of significance:

H₀₁ (null hypotheses): Θ₁≥0 vs. Hₐ₁ (alternative hypotheses): Θ₁<0

Where Θ₁ is the log hazard ratio of PFS in the Lutathera plus Standard Dose octreotide long-acting (30 mg) (investigational) arm vs. High Dose octreotide long-acting (60 mg) (control) arm. The primary efficacy analysis to test this hypothesis and compare the two treatment groups will consist of a stratified log-rank test at an overall one-sided 2.5% level of significance in favor of the Lutathera plus Standard Dose octreotide long-acting (30 mg) arm. The stratification will be based on following randomization stratification factors (grade: G2 vs. G3; and tumor origin: pNET vs other origin).

Analyses will be based on the FAS population according to the randomized treatment group and strata assigned at randomization. The PFS distribution will be estimated using the Kaplan-Meier method, and Kaplan-Meier curves, median and associated 95% confidence intervals will be presented for each treatment group. The hazard ratio for PFS will be calculated, along with its 95% confidence interval, from a stratified Cox model using the same stratification factors as for the log-rank test.

In case of few events in some stratum, the primary analysis will instead be unstratified (the precise handling of strata will be described in the SAP). Both stratified and unstratified analyses will be presented.

### Analysis of Secondary Endpoints

The key secondary objectives in this study are to compare the two treatment groups with respect to:
ORR: rate of patients with best overall response of partial response (PR) or complete response (CR) (centrally assessed according to RECIST 1.1).
Time to deterioration defined as the first deterioration of 10 points (TTD) defined for the EORTC QLQ-C30 and EORTC QLQ-G.I.NET21 global health scale, diarrhea, fatigue, and pain.

Other secondary objectives of the study are to compare the two treatment groups with respect to:
DOR: Duration of Response defined as time from complete or partial response to progression or death due to underlying cancer only.
Rate of adverse events and laboratory toxicities (scored according to CTCAE grade).
OS: time from the randomization date until the day of death due to any cause.

### Key Secondary Objectives

If the primary endpoint is significant, the key secondary endpoints of overall response rate (ORR) and QoL will be tested in a hierarchical fashion to protect the type I error rate. The order of the hypothesis testing shall be ORR followed by QoL Global Health Scale (TTD, see below), followed by QOL Diarrhea (TTD), (TTD)-fatigue, and, (TTD)-pain (TTD). The key secondary objectives will be tested at the time of the primary analysis. At the final analysis, only a descriptive analysis will be performed.

Overall response rate (ORR) is defined as the proportion of subjects with best overall response (BOR) of complete response (CR) or partial response (PR), as per central review and according to RECIST 1.1.

ORR will be calculated based on the FAS and according to the ITT principle. ORR and its 95% confidence interval will be presented by treatment group. The Cochran-Mantel-Haenszel chi-square test, stratified by the randomization stratification factors, will be used to compare ORR between the two treatment groups, at the 1-sided 2.5% level of significance. As a supportive analysis, ORR as per local review will be presented by treatment group, along with 95% confidence intervals.

Time to deterioration (TTD) is defined as time from randomization to the first deterioration of 10 points in domain score compared to the baseline score for the EORTC QLQ-C30 global health scale, diarrhea, fatigue, and pain assessments. The TTD distribution will be estimated using the Kaplan-Meier method, and the Kaplan-Meier curves, medians and 95% confidence intervals of the medians will be presented for each treatment group. Treatment effect will be tested by the stratified log-rank test. The hazard ratio for TTD will be calculated, along with its 95% confidence interval, using a stratified Cox model using stratified the randomization stratification factors. Patients with no baseline and/or no follow-up are censored at randomization. Diarrhea, fatigue and pain are considered clinically relevant (Strosberg, et al., 2018) (Strosberg, et al., 2018), identified as top symptoms in a patient survey (Singh, et al., 2018) (Singh, et al., 2018) and TTD associated with these symptoms domains was significant in a post-hoc analysis of NETTER-1.

Further details regarding the planned sensitivity and supplementary analyses for the key secondary endpoints will be provided in the SAP.

### Other Efficacy Endpoints

Disease Control Rate (DCR) is defined as rate of patients with best overall response of partial response (PR), complete response (CR), or stable disease (SD) (centrally assessed according to RECIST 1.1). More precisely, it counts the presence of at least one confirmed CR or confirmed PR or SD. The DCR will be calculated based on FAS according to the ITT principle. DCR and its 95% confidence interval will be presented by treatment group. The Cochrane-Mantel-Haenszel chi-square test, stratified by the randomization stratification factors, will be used to compare DCR between the two treatment groups, at the one-sided 2.5% level of significance. The p-value to be generated using the Cochrane-Mantel-Haenszel chi-square test is considered nominal, and not to be considered confirmatory as it is not part of the testing strategy. The SAP will advise on how to proceed in case of few events in any stratum.

Duration of response (DOR) only applies to subjects whose best overall response is complete response (CR) or partial response (PR) according to RECIST 1.1 based on tumor response data per local review. The start date is the date of first documented response of CR or PR (i.e., the start date of response, not the date when response was confirmed), and the end date is defined as the date of the first documented progression per local review or death due to underlying cancer. Subjects continuing without progression or death due to underlying cancer will be censored at the date of their last adequate tumor assessment. DOR will be listed and summarized by treatment group for all subjects in the FAS with confirmed BOR of CR or PR.

OS is defined as the time from date of randomization to date of death due to any cause. If a subject is not known to have died, then OS will be censored at the latest date the subject was known to be alive (on or before the cut-off date).

OS will be analyzed in the FAS population according to the randomized treatment group and strata assigned at randomization. At the time of the PFS primary analysis, the OS distribution will be estimated using the Kaplan-Meier method, and the Kaplan-Meier curves, medians and 95% confidence intervals of the medians will be presented for each treatment group. The hazard ratio for OS will be calculated, along with its 95% confidence interval, using a stratified Cox model.

At the final analysis time, a supportive analysis will adjust for cross-over from Control to Lutathera. This will be achieved by using a rank-preserving structural failure time method (RPSFT) to correct for confounding introduced by the change of treatment. The use of the RPSFT method allows survival time estimates gained by anyone receiving Lutathera (i.e. either as randomized to Lutathera or after cross-over from Control to Lutathera). The RPSFT model is based on an accelerated failure time model) and uses a structural assumption of time-proportionality instead of a proportional hazards assumption as used in the Cox model. The widely used Cox model measures drug effect on the hazard ratio scale, whereas the accelerated failure time model measures drug effect on the survival time ratio scale. Additionally, a supportive analysis of OS based on the Inverse Probability Censoring Weight method will be performed at the final analysis time. Further details regarding theses analyses will be given in the SAP.

### Safety Endpoints

For all safety analyses, the safety set will be used. All listings and tables will be presented by treatment group.

The frequency of adverse events and laboratory toxicities (scored according to CTCAE 5.0 grade or current version) will be presented.

All adverse events (AEs), whether or not spontaneously reported by the patient, will be recorded starting from the signing of the ICF until the end of the Treatment Phase. Safety summaries (tables, figures) include only data from the on-treatment period with the exception of baseline data which will also be summarized where appropriate (e.g. change from baseline summaries). In addition, a separate summary for death including on treatment and post treatment deaths will be provided. In particular, summary tables for adverse events (AEs) will summarize only on-treatment events, with a start date during the on-treatment period (treatment-emergent AEs).

The on-treatment period lasts from the date of first administration of study treatment to 30 days after the date of the last actual administration of any study treatment.

The overall observation period will be divided into three mutually exclusive segments:
pre-treatment period: from day of subject's informed consent to the day before first dose of study medication
on-treatment period: from day of first dose of study medication to 30 days after last dose of study medication
post-treatment period: starting at day 30+1 after last dose of study medication.
There will be a dedicated presentation of safety for patients who cross-over.

### Adverse Events

All information obtained on adverse events will be displayed by treatment group and subject. The number (and percentage) of subjects with treatment emergent adverse events (events started after the first dose of study medication or events present prior to start of double-blind treatment but increased in severity based on preferred term) will be summarized in the following ways:
1. by treatment, primary system organ class and preferred term.
2. by treatment, primary system organ class, preferred term and maximum severity.
3. by treatment, Standardized MedDRA Query (SMQ) and preferred term

Separate summaries will be provided for study medication related adverse events, death, serious adverse events, other significant adverse events leading to discontinuation and adverse events leading to dose adjustment.

The number (and proportion) of subjects with AESI/related to identified and potential risks will be summarized by treatment.

A subject with multiple adverse events within a primary system organ class is only counted once towards the total of the primary system organ class.

### Vital Signs

All vital signs data will be listed by treatment group, subject, and visit/time and if ranges are available, abnormalities (and relevant orthostatic changes) will be flagged. Summary statistics will be provided by treatment and visit/time.

### 12-Lead ECG

PR, QRS, QT, QTcF, and RR intervals will be obtained from 12-lead ECGs for each subject during the study. ECG data will be read and interpreted (centrally/locally).

All ECG data will be listed by treatment group, subject and visit/time, abnormalities will be flagged. Summary statistics will be provided by treatment and visit/time.

### Clinical Laboratory Evaluations

All laboratory data will be listed by treatment group, subject, and visit/time and if normal ranges are available abnormalities will be flagged. Summary statistics will be provided by treatment and visit/time. Shift tables using the low/normal/high/(low and high) classification will be used to compare baseline to the worst on-treatment value.

Grading of laboratory values will be assigned programmatically as per NCI Common Terminology Criteria for Adverse Events (CTCAE) version 5.0 or current version. The calculation of CTCAE grades will be based on the observed laboratory values only, clinical assessments will not be taken into account.

CTCAE Grade 0 will be assigned for all non-missing values not graded as 1 or higher. Grade 5 will not be used.

For laboratory tests where grades are not defined by CTCAE, results will be categorized as low/normal/high based on laboratory normal ranges.

The following summaries will be generated separately for hematology, and biochemistry tests:
1. Listing of all laboratory data with values flagged to show the corresponding CTCAE grades if applicable and the classifications relative to the laboratory normal ranges
2. For laboratory tests where grades are defined by CTCAE,
3. Worst post-baseline CTCAE grade (regardless of the baseline status). Each subject will be counted only once for the worst grade observed post-baseline.
4. Shift tables using CTCAE grades to compare baseline to the worst on-treatment value
5. For laboratory tests where grades are not defined by CTCAE,
6. Shift tables using the low/normal/high/(low and high) classification to compare baseline to the worst on-treatment value.

CgA assessment is discontinued in Q3 2022 in South Korea due to lab kits shortage (assessments performed before discontinuation will be included in the analyses).

### Analysis of Exploratory Endpoints

### PFS2

PFS2 is defined as time from date of randomization to the first documented local progression on next-line therapy or death from any cause, whichever occurs first. The first documented progression on next-line treatment is based on investigator assessment of PD (i.e. as captured on the anti-neoplastic therapy after treatment discontinuation eCRF page); it is not necessary to continue to collect tumor assessments data for subsequent anti-neoplastic therapies for the purpose of PFS2.
Next-line therapy is defined as the first new (systemic) anti-neoplastic therapy initiated after discontinuation of study treatment regardless of EoT reason. Drugs given as part of the same regimen should be considered as first line (i.e. part of the next-line therapy).
New anti-neoplastic therapies after EoT will be collected in the anti-neoplastic therapy after treatment discontinuation eCRF page including start/end date, reason for discontinuation, date and type of progression («clinical» vs «radiologic»).
PFS2 will be censored if no PFS2 event (progression or death) is observed during next-line therapy before the analysis cut-off date; the censoring date will be the last contact date.
However, in case a second new anti-neoplastic therapy is introduced without prior PFS2 event, then PFS2 will be censored at the end date of the first new anti-neoplastic therapy (i.e. next line therapy).
Any death prior to initiation of next-line therapy will be considered as an event for PFS2. Any death occurring following end of next line therapy will be considered as an event if no second new anti-neoplastic therapy has been introduced.
PFS and PFS2 may be identical if a subject did not experience an event (i.e. progression) prior to initiation of next-line therapy, and adequate tumor assessments continue until RECIST 1.1-documented disease progression after initiation of next-line therapy.

PFS2 will be analyzed in the FAS population according to the randomized treatment group and strata assigned at randomization. The PFS2 distribution will be estimated using the Kaplan-Meier method, and the Kaplan-Meier curves, medians and 95% confidence intervals of the medians will be presented for each treatment group. The hazard ratio for PFS2 will be calculated, along with its 95% confidence interval, using a stratified Cox model.

Time to Second Progression (PFS2) in the two treatment arms will be evaluated as a secondary endpoint. This endpoint is defined as the time in months from the first progression to the second progression or death. The date of both the first and second progression will be derived programmatically through the RECIST criteria.

It is recognized that due to cross-over, the lower scan frequency (every six months) after PEP, and, the relatively short follow-up, the possibility to draw firm conclusions is limited.

Patient Reported Outcomes not Associated with Key Secondary Endpoint

TTD for items/scales derived from EORTC QLQ-G.I.NET21 and EORTC QLQ-30 not included among the key secondary endpoints will be analyzed as exploratory endpoints. The TTD distribution will be estimated using the Kaplan-Meier method, and the Kaplan-Meier curves, medians and 95% confidence intervals of the medians will be presented for each treatment group. The hazard ratio for TTD will be calculated, along with its 95% confidence interval, using a stratified Cox model.

Quality of Life will also be assessed in terms of change from baseline in EORTC QLQ-G.I.NET21, EORTC QLQ-C30 and EQ-5D-5L through Mixed Model Repeated Measures (MMRM), which deals with missing values in a model based way, and implicitly imputes missing data under a missing at random assumption. The model will include the explanatory variables Treatment, Baseline, Week, and, the interaction between Treatment and Week. A test of treatment effect is performed at each week, and, overall.

### Pharmacokinetics

Patients in the control arm (all countries except for China) will yield PK data from blood sampling performed at pre-dose with respect to the octreotide long-acting i.m. injection for the 2nd, 4th, 5th and 7th treatment cycle (that is respectively at week 4, 12, 16 and 24) for the determination of plasma trough levels. Data will be processed using a population based model, for exploratory purposes. More details on the presentation of results will be given in the SAP.

Additionally, descriptive statistics and graphical display of octreotide plasma concentrations during the course of treatment will presented by dose group. Exploratory analysis will be performed to assess the dose-exposure relationship. More details on the presentation of results will be given in the SAP.

Re-Treatment with Lutathera

Safety will be presented in a similar manner to that for those participants who receive re-treatment with Lutathera during the re-treatment period. The re-treatment period lasts from the date of first administration of re-treatment with Lutathera to progression or death during the re-treatment period.

All information obtained on adverse events will be displayed by subject.

All vital signs data will be listed subject, and visit/time and if ranges are available, abnormalities (and relevant orthostatic changes) will be flagged. Summary statistics will be provided by visit/time.

All ECG data (as per local assessment) will be listed by subject and visit/time, abnormalities will be flagged. Summary statistics will be provided by visit/time.

All laboratory data will be listed by subject, and visit/time and if normal ranges are available abnormalities will be flagged. Summary statistics will be provided by visit/time. Shift tables using the low/normal/high/(low and high) classification will be used to compare the latest assessment before re-treatment to the worst re-treatment value.

All efficacy analyses for ORR, DoR, PFS, PFS2, and OS will be descriptive. Definitions for ORR, DoR, and PFS (as per local assessment) are the same definitions except that the baseline will be based on the tumor assessment which demonstrated progression thus allowing re-treatment with Lutathera. Definitions for OS and PFS2 are the same to that in described herein, respectively.

### Interim Analyses

An interim analysis with respect to OS will be performed at the time of the PFS final analysis. At this time point, only estimation of treatment effect will be performed (hazard ratio and its 95% confidence interval).

### Sample Size Calculation

### Primary Endpoint(s)

The sample size calculation is based on the primary variable PFS. Assuming a median PFS in the control arm (High Dose octreotide long-acting (60 mg)) of approximately 15 months, it is hypothesized that treatment with Lutathera added to standard dose octreotide long-acting will result in a 50% reduction in the hazard rate (corresponding to an increase in median PFS from 15 months to 30 months).

To ensure 90% power to test the null hypothesis: PFS hazard ratio=1, versus the specific alternative hypothesis: PFS hazard ratio=0.50, it is calculated that a total of 99 PFS events need to be observed. This calculation assumes analysis by a one-sided log-rank test at the overall 2.5% level of significance, patients randomized to the two treatment groups in a 2:1 ratio. Assuming that enrolment will continue for approximately 22.2 months at a rate of 10 patients per month and a 15% dropout rate by the time of the final PFS analysis, a total of 222 patients (148 for Lutathera arm and 74 for the control arm) will need to be randomized to observe the targeted 99 PFS events at about 12.8 months after the randomization date of the last patient, i.e., 35 months after the randomization date of the first patient. If the final analysis is performed when the targeted 99 PFS events are observed, the observed hazard ratio will have to be <0.658 which corresponds to a difference in median PFS of 7.8 months to declare statistical significance.

These calculations were made using the software package East 6.4.

Randomization will be stratified by Grade (G2 vs G3) and tumor origin (pNET vs other origin).

### DETAILED EMBODIMENTS

The methods of treatments of the present disclosure are provided in particular as following embodiments:

A method of treating well-differentiated G2 or G3 neuroendocrine tumor (NET) in a patients in need thereof, comprising administering to said patient a treatment comprising a therapeutically effective dose of a somatostatin receptor (SSTR) targeting radioligand therapeutic (RLT) agent as first line therapy.

A method of delaying the time-to-first-occurrence progression or death in a patient with well-differentiated G2 or G3 neuroendocrine tumor (NET), comprising administering to said patient a treatment comprising a therapeutically effective dose of a somatostatin receptor (SSTR) targeting radioligand therapeutic (RLT) agent as first line therapy.

A method of delaying the need for the initiation of chemotherapy in a patient with well-differentiated G2 or G3 neuroendocrine tumor (NET), comprising administering to said patient a treatment comprising a therapeutically effective dose of a somatostatin receptor (SSTR) targeting radioligand therapeutic (RLT) agent as first line therapy.

A method of controlling the disease in a patient with well-differentiated G2 or G3 neuroendocrine tumor (NET), comprising administering to said patient a treatment comprising a therapeutically effective dose of a somatostatin receptor (SSTR) targeting radioligand therapeutic (RLT) agent as first line therapy.

A method of remitting well-differentiated G2 or G3 neuroendocrine tumor (NET) in a patient in need thereof, comprising administering to said patient a treatment comprising a therapeutically effective dose of a somatostatin receptor (SSTR) targeting radioligand therapeutic (RLT) agent as first line (1L) therapy.

A method of reducing the risk of progression of well-differentiated G2 or G3 neuroendocrine tumor (NET) in a patient in need thereof or reducing the risk of death of said patient, comprising administering to said patient a treatment comprising a therapeutically effective dose of a somatostatin receptor (SSTR) targeting radioligand therapeutic (RLT) agent as first line (1L) therapy.

In certain embodiments, the RLT agent comprises a radionuclide selected from the group consisting of: 47Sc, 67Cu, 153Sm, 161Tb, 169Er, and 177Lu.

In certain embodiments, the RLT agent comprises a radionuclide selected from the group consisting of: 161Tb and 177Lu.

In certain embodiments, the RLT agent comprises a radionuclide selected from the group consisting of: 161Tb and 177Lu.

In certain embodiments, the RLT agent comprises the radionuclide 177Lu.

In certain embodiments, the 177Lu is no carrier added (n.c.a.) 177Lu or carrier-added (c.a.) 177Lu.

In certain embodiments, the 177Lu is n.c.a. 177Lu (also referred to as NCA 177Lu).

In certain embodiments, the 177Lu is carrier-added (c.a.) 177Lu (also referred to as CA 177Lu).

In certain embodiments, the RLT agent comprises a ligand selected from the group consisting of: oxodotreotide (DOTATATE), edotreotide (DOTATOC), and satoreotide tetraxetan.

In certain embodiments, the RLT agent comprises a ligand selected from the group consisting of: oxodotreotide (DOTATATE) and edotreotide (DOTATOC).

In certain embodiments, the RLT agent comprises the ligand oxodotreotide (DOTATATE).

In certain embodiments, the RLT agent is selected from the group consisting of: lutetium (177Lu) oxodotreotide, lutetium (177Lu) edotreotide, lutetium (177Lu) satoreotide tetraxetan, terbium (161Tb) oxodotreotide, terbium (161Tb) edotreotide, and terbium (161Tb) satoreotide tetraxetan.

In certain embodiments, the RLT agent is selected from the group consisting of: lutetium (177Lu) oxodotreotide, lutetium (177Lu) edotreotide, terbium (161Tb) oxodotreotide, and terbium (161Tb) edotreotide.

In certain embodiments, the RLT agent is selected from the group consisting of: lutetium (177Lu) oxodotreotide and lutetium (177Lu) edotreotide.

In certain embodiments, the RLT agent is lutetium (¹⁷⁷Lu) oxodotreotide (also referred to as ¹⁷⁷Lu[Lu]-DOTA-(Tyr³)-octreotate, or ¹⁷⁷Lu[Lu]-DOTA-0-Tyr3-octreotate).

In certain embodiments, the dose of the RLT agent is from 5 to 10 GBq, from 6 to 9 GBq, from 6.5 to 8.5 GBq, 7.4±10% GBq, or 7.5±0.7 GBq.

In certain embodiments, said dose is administered once every 40 to 100 days, once every 49 to 90 days, once every 90 days, or once every 8±1 weeks.

In certain embodiments, said dose administered up to 4 times (4 cycles).

In certain embodiments, the RLT agent is administered at a dose of 7.4±10% GBq every 8±1 weeks for up to 4 times (4 cycles).

In certain embodiments, the patient after the initial first up to 4 cycles is re-treated by 2 further cycles with optionally further 2 cycles (2+opt. 2).

In certain embodiments, the treatment further comprises a supportive care.

In certain embodiments, the supportive care is 30 mg long-acting octreotide (e.g. octreotide LAR) (as available under the drug product name Sandostatin LAR) once every month.

In certain embodiments, the supportive care is 30 mg long-acting octreotide (e.g. octreotide LAR, e.g. as available under the drug product name Sandostatin LAR) once every 4 weeks.

In certain embodiments, the supportive care is 30 mg long-acting octreotide (e.g. octreotide LAR, e.g. as available under the drug product name Sandostatin LAR) once every 8 weeks.

In certain embodiments, the supportive care is 30 mg long-acting octreotide (e.g. octreotide LAR, e.g. as available under the drug product name Sandostatin LAR) once every eight weeks during the treatment with the treatment with the RLT agent. In certain embodiments the long-acting octreotide (e.g. octreotide LAR) is (then) adminstered once every 4 weeks after the last treatment with the RLT agent.

In certain embodiments, the RLT agent is ¹⁷⁷Lu-DOTA-TATE administered at a dose of 7.4±10% GBq every 8±1 weeks for up to 4 times (4 cycles) and the treatment further comprises treatment with 30 mg octreotide LAR once every 8 weeks during treatment with the ¹⁷⁷Lu-DOTA-TATE. In an embodiment, the octreotide LAR is administered once every 4 weeks after the 4 cycles of treatment of the ¹⁷⁷Lu-DOTA-TATE is completed.

In certain embodiments, the RLT agent is ¹⁷⁷Lu-DOTA-TATE administered at a dose of 7.4±10% GBq every 8±1 weeks for up to 4 times (4 cycles) and the treatment further comprises treatment with 30 mg octreotide LAR 4 to 24 hours after each administration of ¹⁷⁷Lu-DOTA-TATE. In an embodiment, the octreotide LAR is administered once every 4 weeks after the 4 cycles of treatment of the ¹⁷⁷Lu-DOTA-TATE is completed.

In certain embodiments, the treatment further comprises the administration of a 2.5% Lysine-Arginine solution intravenously (IV).

In certain embodiments, the treatment is characterized by a centrally assessed progression free survival (PFS) of at least 18, 20, or 22 months. In certain embodiments, the treatment is characterized by a centrally assessed progression free survival (PFS) of at least 18, 19, 20, 21, or 22 months. In certain embodiments, the treatment is characterized by a centrally assessed progression free survival (PFS) of at least 18, 19, 20, 21, 22, 23, 24, or 25 months. In certain embodiments, the treatment is characterized by a centrally assessed progression free survival (PFS) of at least 20, 21, 22, 23, 24, 25, 26, 27, 28, 29, or 30 months. In certain embodiments, the treatment is characterized by a centrally assessed progression free survival (PFS) of at least 18, 24, 30, 36, 42, or 48 months.

In certain embodiments, the progression is radiological progression according to RECIST (v.1.1) based on central assessment.

In certain embodiments, the treatment is characterized by a disease control rate (DCR, i.e. complete response (CR) rate+partial response (PR) rate)+stable disease (SD) rate) of at least 50, 60, 70, 80, 85, 86, 87, 88, 89, or 90%.

In certain embodiments, the treatment is characterized by a objective response rate (ORR, i.e. complete response (CR) rate+partial response (PR) rate) of at least 25, 30, 35, 40, 41, 42, or 43%.

In certain embodiments, the treatment reduces the risk of progression or death by at least 50, 60, 65, 70, 72% compared to a comparable treatment without the RLT agent.

In certain embodiments, the treatment reduces the risk of progression or death by at least 50, 60, 65, 66, 67, 68, 69, 70, 71, 72, 73, 74, 75% compared to a comparable treatment without the RLT agent.

In certain embodiments, the treatment reduces the risk of progression or death corresponding to a hazard ratio (HR) of less than 0.5, 0.4, 0.35, 0.3, 0.28 (95% CI) when the risk is calculated relative to patients a comparable treatment without the RLT agent.

In certain embodiments, the treatment reduces the risk of progression or death corresponding to a hazard ratio (HR) of less than 0.5, 0.4, 0.35, 0.34, 0.33, 0.32, 0.32, 0.30, 0,29, 0.28, 0.27, 0.26, 0.25 when the risk is calculated relative to patients a comparable treatment without the RLT agent.

In certain embodiments, the comparable treatment without the RLT agent comprises administration of a drug selected from the group consisting of: octreotide, e.g. high dose long-acting octreotide (e.g. 60 mg long-acting octreotide (octreotide LAR)), lanreotide, everolimus, interferon-alpha (IFN-alpha), sunitinib, surufatinib, lenvatinib, streptozotocin (STZ), etoposide, irinotecan, and chemotherapies (e.g. comprising capecitabine, temozolomide, dacarbazine, folinic acid, 5-fluorouracil (5FU), and platinum-based chemotherapies (e.g. oxaliplatin, cisplatin)) or combinations thereof (e.g. STZ-5FU, CAPTEM, CAP-5FU, FOLFOX, FOLFIRI); preferably: 60 mg long-acting octreotide (octreotide LAR), CAPTEM (capecitabine and temozolomide), Everolimus, and FOLFOX (folinic acid, fluorouracil, and oxaliplatin); more preferably: high dose long-acting octreotide (e.g. 60 mg long-acting octreotide (octreotide LAR))

In certain embodiments, the comparable treatment without the RLT agent is 60 mg long-acting octreotide (octreotide LAR, e.g. as available as Sandostatin LAR).

In certain embodiments, the treatment is characterized by treatment-related Grade ≥3 adverse events (AE) of not more than 30, 25, 20, 18, or 16%.

In certain embodiments, the treatment is characterized by treatment-related Grade ≥3 serious adverse events (SAE) of not more than 10, 9, 8, 7, 6, or 5%.

In certain embodiments, the patient is ≥15 years old.

In certain embodiments, the patient is newly diagnosed of for advanced G2 or G3 GEP-NET.

In certain embodiments, the patient is newly diagnosed of for advanced G2 GEP-NET.

In certain embodiments, the patient is newly diagnosed of for advanced G3 GEP-NET.

In certain embodiments, the patient is newly diagnosed of for advanced G2 GEP-NET and the progression/death risk reduction corresponds to a HR of 0.31 or less.

In certain embodiments, the patient is newly diagnosed of for advanced G3 GEP-NET and the progression/death risk reduction corresponds to a HR of 0.27 or less.

In certain embodiments, the median time to deterioration by 10 points in the global health status in the EORTC QLQ-C30 (European Organization for the Research and Treatment of Cancer Quality of Life Questionnaire) is at least 9 months, at least 10 months, at least 11 months, at least 12 months or at least 13 months.

In an embodiment, the overall response rate is at least 10%. In an embodiment, the overall response rate is at least 15%. In an embodiment, the overall response rate is at least 20%. In an embodiment, the overall response rate is at least 25%. In an embodiment, the overall response rate is at least 30%. In an embodiment, the overall response rate is at least 35%. In an embodiment, the overall response rate is at least 40%.

In an embodiment, the NET has metastasized to the bone.

In an embodiment, the NET has metastasized to the liver.

In an embodiment, the NET has metastasized to the lymph nodes.

In an embodiment, the NET has metastasized to the peritoneum.

In an embodiment, Chromogranin A (CgA) prior to treatment with the somatostatin receptor (SSTR) targeting radioligand therapeutic (RLT) is >2. In an alternative embodiment, the CgA prior to treatment with the somatostatin receptor (SSTR) targeting radioligand therapeutic (RLT) is ≤2.

According to an aspect of the invention, there is hereby provided a method of reducing the incidence of a cardiovascular or electrolyte disorder in a patient having a well-differentiated G2 or G3 neuroendocrine tumor (NET) (e.g. as compared to treatment with 60 mg octreotide LAR once every 4 weeks), the method comprising administering to said patient ¹⁷⁷Lu-DOTA-TATE as first line therapy in combination with octreotide LAR.

In an embodiment, the RLT agent is ¹⁷⁷Lu-DOTA-TATE, and the ¹⁷⁷Lu-DOTA-TATE is administered at a dose providing 7.4 GBq/200 mCi±10% every 8±1 weeks. In an embodiment, a total of 4 doses of the ¹⁷⁷Lu-DOTA-TATE are adminstered. In an embodiment, the octreotide LAR is administered at a dose of 30 mg every 8 weeks during treatment with the ¹⁷⁷Lu-DOTA-TATE and then at a dose of 30 mg every 4 weeks after the last treatment with the ¹⁷⁷Lu-DOTA-TATE.

According to an aspect of the invention, there is hereby provided a method of both i) reducing the incidence of a cardiovascular or electrolyte disorder and ii) a) increasing the median PFS and/or b) the overall response rate in a patient having a well-differentiated G2 or G3 neuroendocrine tumor (NET) as compared to treatment with 60 mg octreotide LAR once every 4 weeks, the method comprising administering to said patient 177Lu-DOTA-TATE as first line therapy in combination with 30 mg octreotide LAR. In an embodiment, the dose of 177Lu-DOTA-TATE is 7.4 GBq/200 mCi±10% administered every 8±1 weeks. In an embodiment, a total of 4 doses of the ¹⁷⁷Lu-DOTA-TATE are adminstered. In an embodiment, the octreotide LAR is administered at 30 mg every 8 weeks during treatment with the ¹⁷⁷Lu-DOTA-TATE and at 30 mg every 4 weeks after the last treatment with the ¹⁷⁷Lu-DOTA-TATE.

In certain embodiments, the 1L therapy is characterized by no previous treatments with interferons, mTOR inhibitors, or chemotherapeutic agents.

In certain embodiments, the 1L therapy is characterized by no previous treatments with sandostatin analogues (SSA-naïve), or in case of a previous treatment with SSA the patient is non-progressive under said SSA therapy.

In certain embodiments, the NET is a gastroentero-pancreatic NET (GEP-NET).

In certain embodiments, the NET is a pancreatic NET (pNET, i.e. the tumor origin is in the prancreas).

In certain embodiments, the NET is a pancreatic NET (pNET) and the progression/death risk reduction corresponds to a HR of 0.34 or less.

In certain embodiments, the NET is a small intestine NET (i.e. primary site of cancer is in the small intestine).

In certain embodiments, the NET is a small intestine NET and the progression/death risk reduction corresponds to a HR of 0.30 or less.

In certain embodiments, the NET is an advanced NET.

In certain embodiments, the NET is metastasized or locally advanced and inoperable.

In certain embodiments, the NET is SSTR-positive (SSTR+).

In certain embodiments, the SSTR-positivity of the NET is determined by CT or MRI scans with a radioligand imaging (RLI) agent.

In certain embodiments, the SSR-positivity of the NET is determined by PET/CT, SPECT/CT or SRS (somatostatin receptor scintigraphy) with a radioligand imaging (RLI) agent.

In certain embodiments, the RLI agent is selected from the group consisting of: 68Ga-oxodotreotide, 68Ga-edotreotide, 68Ga-satoreotide trizoxetan, 64Cu-oxodotreotide, 64Cu-edotreotide, 64Cu-satoreotide trizoxetan, 111 In-pentetreotide, and 99mTc-tektrotyd.

The drug [177Lu]Lu-DOTATATE, INN: lutetium (177Lu) oxodotreotide, is available as drug product under the name LUTATHERA. The drug [177Lu]Lu-DOTATOC, INN: lutetium (¹⁷⁷Lu) edotreotide, [177Lu-DOTA(0), Tyr(3)]-octreotide (DOTATOC, where DOTA tetraazacyclododecane tetraacetic acid and TOC=D-Phe-c(Cys-Tyr-D-Trp-Lys-Thr-Cys)-Thr(ol)), can be produced to a drug product according to WO 2022/111800 A1 and/or WO 2022/112323 A1 as is in use under the drug product name SOLUCIN or ITM-11. The drug lutetium (¹⁷⁷Lu) satoreotide tetraxetan may be produced to a drug product in a similar way as Lutathera or Solucin.

In certain embodiments, the RLT agent may also comprise as SSTR binder other somatostatin analogues, e.g. octreotide, octreotate, satoreotide, lanreotide, vapreotide, and pasireotide. Together with the DOTA chelator it will form the corresponding ligand, e.g. DOTA-OC, DOTA-TOC (edotreotide), DOTA-NOC, DOTA-TATE (oxodotreotide), satoreotide tetraxetan, DOTA-LAN, and DOTA-VAP.

In certain embodiments, the RLT agent may comprise a chelator selected from the group consisting of: DOTA (tetraxetan), trizoxetan, DOTAGA, DTPA, NTA, EDTA, DO3A, TETA, NOTA, NOTAGA, NODAGA, NODAPA, PCTA and AAZTA (e.g. AAZTA5).

In certain embodiments, the RLT agent may comprise an albumin binder, such as Evans blue (EB), e.g. [177Lu]Lu-DOTA-EB-TATE.

### Medical Use Formats

The embodiments above are formulated as method of treatment claims. They may be likewise be formulated into other second medical use formats, such as illustrated in the following, with DRUG being the RLT agent (alone or in combination with other agents), with INDICATION being NET as specified in the embodiments above, with FEATURES being those characterizing features as mentioned in the embodiments above:

The present invention provides [DRUG] or any pharmaceutically acceptable salt thereof for use in the treatment of [INDICATION], characterized in that [FEATURES].

Alternatively, the present invention provides a method for the treatment of [INDICATION] in human patients in need of such treatment which comprises administering an effective amount of [DRUG] or any pharmaceutically acceptable salt thereof, wherein said treatment is characterized in that [FEATURES].

As a further alternative the present invention provides the use of [DRUG] or any pharmaceutically acceptable salt thereof for the preparation of a medicament for the treatment of [INDICATION], characterized in that [FEATURES].

As a further alternative the present invention provides the use of [DRUG] or any pharmaceutically acceptable salt thereof for the treatment of [INDICATION], characterized in that [FEATURES].

As a further alternative the present invention provides a medicament for the treatment of [INDICATION] comprising [DRUG] or any pharmaceutically acceptable salt thereof, characterized in that [FEATURES].

### FURTHER DEFINITIONS

“about” in respect of a value means±25%, preferably +20%, more preferably ±15%, even more preferably ±10%, even more preferably ±5%.
“about” in respect of a weeks or cycles means±2, preferably ±1.

The use of the articles “a”, “an”, and “the” in both the description and claims are to be construed to cover both the singular and the plural, unless otherwise indicated herein or clearly contradicted by context. The terms “comprising”, “having”, “being of” as in e.g., a complex “of a radionuclide and a cell receptor binding organic moiety linked to a chelating agent”, “including”, and “containing” are to be construed as open terms (i.e., meaning “including but not limited to”) unless otherwise noted. Additionally, whenever “comprising” or another open-ended term is used in an embodiment, it is to be understood that the same embodiment can be more narrowly claimed using the intermediate term “consisting essentially of” or the closed term “consisting of”.

The term “about” or “ca.” has herein the meaning that the following value may vary for ±20%, preferably ±10%, more preferably ±5%, even more preferably ±2%, even more preferably ±1%.
“about” in respect of a value means±25%, preferably ±20%, more preferably ±15%, even more preferably ±10%, even more preferably ±5%.
“about” in respect of a weeks or cycles means±2, preferably ±1.

The phrase “treatment of” and “treating” includes the prevention, the amelioration or cessation of a disease, disorder, or a symptom thereof. In particular, with reference to the treatment of a tumor, the term “treatment” may refer to the inhibition of the growth of the tumor, or the reduction of the size of the tumor.

Consistent with the International System of Units, “MBq” is the abbreviation for the unit of radioactivity “megabecquerel.”

Consistent with the International System of Units, “GBq” is the abbreviation for the unit of radioactivity “gigabecquerel.”

The terms radionuclide and radioisotopes can be exchanged in this disclosure and its embodiments. Where it is technically meaningful, the term radiometal and radiohalogen can be also used as alternative.

Instead of indicating the radionuclide in the form of e.g. ¹⁷⁷Lu or Lu-177 or Lutetium-177, the form 177Lu is used as alternative herein. Therefore, wherever the mass number, e.g. 177, is not superscripted (e.g. 177Lu) throughout the disclosure, it is nevertheless meant to refer to the superscripted version ¹⁷⁷Lu.

As used herein, “PET” stands for positron-emission tomography.

As used herein, “SPECT” stands for single-photon emission computed tomography.

As used herein, “MRI” stands for magnetic resonance imaging.

As used herein, “CT” stands for computed tomography.

As used herein, the terms “efficient amount” or “therapeutically efficient amount” of a compound refer to an amount of the compound that will elicit the biological or medical response of a subject, for example, ameliorate the symptoms, alleviate conditions, slow or delay disease progression, or prevent a disease.

The terms “patient” and “subject” which are used interchangeably refer to a human being, including for example a subject that has cancer.

As used herein the term “radiopharmaceutical” refers to a pharmaceutical compound which is labelled with a radionuclide element, typically of metallic nature. A radiopharmaceutical compound may be used in peptide receptor radionclide therapy (PRRT).

As used herein, the term “PRRT” or “peptide receptor radionclide therapy” refers to a molecularly targeted radiation therapy involving the systemic administration of a radiolabeled peptide (e.g., ¹⁷⁷Lu-dotatate), which is designed to target receptors overexpressed on tumors (e.g., somatostatin receptor subtype 2) with high affinity and specificity.

DOTA-OC: [DOTA⁰,D-Phe¹] octreotide,

DOTA-TOC: [DOTA⁰,D-Phe¹,Tyr³] octreotide, edotreotide (INN), represented by the following formulas:

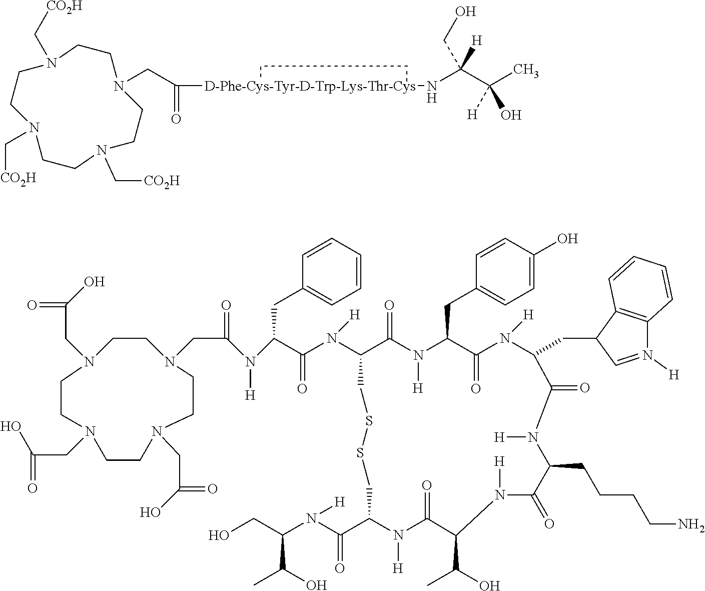

DOTA-NOC: [DOTA⁰, D-Phe¹, 1-Nal³]octreotide,
DOTA-TATE: [DOTA⁰,D-Phe¹,Tyr³] octreotate, DOTA-Tyr³-Octreotate, DOTA-d-Phe-Cys-Tyr-d-Trp-Lys-Thr-Cys-Thr (cyclo 2,7), oxodotreotide (INN), represented by the following formula:

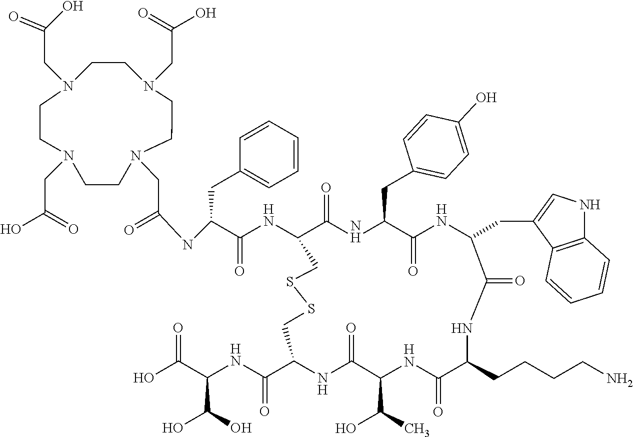

DOTA-LAN: [DOTA⁰,D-β-Nal¹] lanreotide,
DOTA-VAP: [DOTA⁰,D-Phe¹,Tyr³] vapreotide.

### Satoreotide Trizoxetan

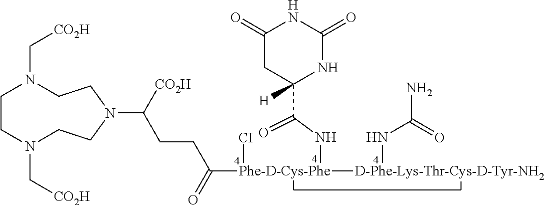

### Satoreotide Tetraxetan

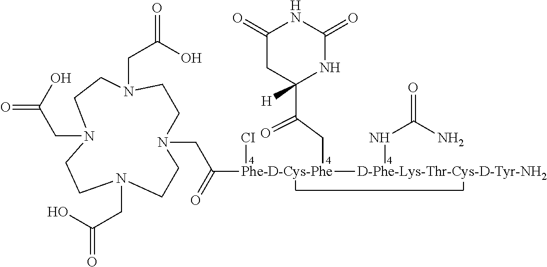

### REFERENCES

All publications, patents and patent applications referred to herein are incorporated by reference in their entirety to the same extent as if each individual publication, patent or patent application was specifically and individually indicated to be incorporated by reference in its entirety.
Anthony, L., & Vinik, A. I. (2011, 10). Evaluating the characteristics and the management of patients with neuroendocrine tumors receiving octreotide LAR during a 6-year period. Pancreas, 40 (7), 987-994. doi: 10.1097/MPA.0b013e31821f66b4
Anthony, S. S. (2004, 7). Octreotide doses used in clinical practice: Results from an internet survey and a clinical Practice. Journal of Clinical Oncology, 22, 4274.
Astruc, B., Marbach, P., Bouterfa, H., Denot, C., Safari, M., Vitaliti, A., & Sheppard, M. (2005, 7). Long-acting octreotide and prolonged-release lanreotide formulations have different pharmacokinetic profiles. Journal of clinical pharmacology, 45 (7), 836-844. doi: 10.1177/0091270005277936
Barani, I. J., & Larson, D. A. (2015). Radiation therapy of glioblastoma. Cancer treatment and research, 163, 49-73. doi: 10.1007/978-3-319-12048-5_4
Bergsma, H., Konijnenberg, M. W., Kam, B. L., Teunissen, J. J., Kooij, P. P., Herder, W. W., . . . Kwekkeboom, D. J. (2016a, 3). Subacute haematotoxicity after PRRT with (177)Lu-DOTA-octreotate: prognostic factors, incidence and course. European journal of nuclear medicine and molecular imaging, 43 (3), 453-463. doi: 10.1007/s00259-015-3193-4
Bergsma, H., Konijnenberg, M. W., Zwan, W. A., Kam, B. L., Teunissen, J. J., Kooij, P. P., . . . Kwekkeboom, D. J. (2016b, 9). Nephrotoxicity after PRRT with (177) Lu-DOTA-octreotate. European journal of nuclear medicine and molecular imaging, 43 (10), 1802-1811. doi: 10.1007/s00259-016-3382-9
Brabander, T., Teunissen, J. J., Van Eijck, C. H., Franssen, G. J., Feelders, R. A., Herder, W. W., & Kwekkeboom, D. J. (2016, 1). Peptide receptor radionuclide therapy of neuroendocrine tumours. Best practice & research. Clinical endocrinology & metabolism, 30 (1), 103-114. doi: 10.1016/j.beem.2015.10.005
Brabander, T., Zwan, W. A., Teunissen, J. J., Kam, B. L., Feelders, R. A., Herder, W. W., Kwekkeboom, D. J. (2017a, 8). Long-Term Efficacy, Survival, and Safety of [177Lu-DOTA0,Tyr3] octreotate in Patients with Gastroenteropancreatic and Bronchial Neuroendocrine Tumors. Clinical cancer research: an official journal of the American Association for Cancer Research, 23(16), 4617-4624. doi: 10.1158/1078-0432.CCR-16-2743
Brabander, T., Zwan, W. A., Teunissen, J. J., Kam, B. L., Herder, W. W., Feelders, R. A., . . . Kwekkeboom, D. J. (2017b, 5). Pitfalls in the response evaluation after peptide receptor radionuclide therapy with [177Lu-DOTA0,Tyr³] octreotate. Endocrine-related cancer, 24 (5), 243-251. doi: 10.1530/ERC-16-0524
Broder, M. S., Beenhouwer, D., Strosberg, J. R., Neary, M. P., & Cherepanov, D. (2015, 2). Gastrointestinal neuroendocrine tumors treated with high dose octreotide-LAR: a systematic literature review. World journal of gastroenterology, 21 (6), 1945-1955. doi: 10.3748/wjg.v21.i6.1945
Caplin, M. E., Pavel, M., Ćwikla, J. B., Phan, A. T., Raderer, M., Sedláčková, E., . . . Investigators, C. L. (2014, 7). Lanreotide in metastatic enteropancreatic neuroendocrine tumors. The New England journal of medicine, 371 (3), 224-233. doi: 10.1056/NEJMoa1316158
Catena, L., Bichisao, E., Milione, M., Valente, M., Platania, M., Pusceddu, S., . . . Bajetta, E. (2011). Neuroendocrine tumors of unknown primary site: gold dust or misdiagnosed neoplasms? Tumori, 97 (5), 564-567. doi: 10.1700/989.10712
Chadha, M. K., Lombardo, J., Mashtare, T., Wilding, G. E., Litwin, A., Raczyk, C., . . . Iyer, R. V. (2009, 10). High-dose octreotide acetate for management of gastroenteropancreatic neuroendocrine tumors. Anticancer research, 29 (10), 4127-4130.
Chen, T., Miller, T. F., Prasad, P., Lee, J., Krauss, J., Miscik, K., . . . McLeod, J. F. (2000, 5). Pharmacokinetics, pharmacodynamics, and safety of microencapsulated octreotide acetate in healthy subjects. Journal of clinical pharmacology, 40 (5), 475-481.
Clinical Trial Facilitation Group (CTFG) 2020: Recommendations related to contraception and pregnancy testing in clinical trials. Version 1.1, September 2020.
Coriat, R., Walter, T., Terris, B., Couvelard, A., & Ruszniewski, P. (2016, 10). Gastroenteropancreatic Well-Differentiated Grade 3 Neuroendocrine Tumors: Review and Position Statement. The oncologist, 21 (10), 1191-1199. doi: 10.1634/theoncologist.2015-0476
Dasari, A., Shen, C., Halperin, D., Zhao, B., Zhou, S., Xu, Y., . . . Yao, J. C. (2017, 10). Trends in the Incidence, Prevalence, and Survival Outcomes in Patients With Neuroendocrine Tumors in the United States. JAMA oncology, 3 (10), 1335-1342. doi: 10.1001/jamaoncol.2017.0589
Dhall, D., Mertens, R., Bresee, C., Parakh, R., Wang, H. L., Li, M., . . . Wolin, E. (2012, 4). Ki-67 proliferative index predicts progression-free survival of patients with well-differentiated ileal neuroendocrine tumors. Human pathology, 43 (4), 489-495. doi: 10.1016/j.humpath.2011.06.011
Eisenhauer, E. A., Therasse, P., Bogaerts, J., Schwartz, L. H., Sargent, D., Ford, R., . . . Verweij, J. (2009, 1). New response evaluation criteria in solid tumours: revised RECIST guideline (version 1.1). European journal of cancer (Oxford, England: 1990), 45 (2), 228-247. doi: 10.1016/j.ejca.2008.10.026
Eriksson, B., Renstrup, J., Imam, H., & Oberg, K. (1997, 10). High-dose treatment with lanreotide of patients with advanced neuroendocrine gastrointestinal tumors: clinical and biological effects. Annals of oncology: official journal of the European Society for Medical Oncology, 8 (10), 1041-1044.
Faiss, S., Pape, U.-F., Bohmig, M., Dorffel, Y., Mansmann, U., Golder, W., . . . Group, I. A. (2003, 7). Prospective, randomized, multicenter trial on the antiproliferative effect of lanreotide, interferon alfa, and their combination for therapy of metastatic neuroendocrine gastroenteropancreatic tumors—the International Lanreotide and Interferon Alfa Study Group. Journal of clinical oncology: official journal of the American Society of Clinical Oncology, 21 (14), 2689-2696. doi: 10.1200/JCO.2003.12.142
Faiss, S., Räth, U., Mansmann, U., Caird, D., Clemens, N., Riecken, E. O., & Wiedenmann, B. (1999). Ultra-high-dose lanreotide treatment in patients with metastatic neuroendocrine gastroenteropancreatic tumors. Digestion, 60 (5), 469-476. doi: 10.1159/000007693
Ferolla, P., Faggiano, A., Grimaldi, F., Ferone, D., Scarpelli, G., Ramundo, V., . . . Colao, A. (2012, 3). Shortened interval of long-acting octreotide administration is effective in patients with well-differentiated neuroendocrine carcinomas in progression on standard doses. Journal of endocrinological investigation, 35 (3), 326-331. doi: 10.3275/7869
Forrer, F., Uusijärvi, H., Storch, D., Maecke, H. R., & Mueller-Brand, J. (2005, 8). Treatment with ¹⁷⁷Lu-DOTATOC of patients with relapse of neuroendocrine tumors after treatment with 90Y-DOTATOC. Journal of nuclear medicine: official publication, Society of Nuclear Medicine, 46 (8), 1310-1316.
Halperin, D. M., Shen, C., Dasari, A., Xu, Y., Chu, Y., Zhou, S., . . . Yao, J. C. (2017, 4). Frequency of carcinoid syndrome at neuroendocrine tumour diagnosis: a population-based study. The Lancet. Oncology, 18 (4), 525-534. doi: 10.1016/S1470-2045 (17) 30110-9
Hammond, P. J., Wade, A. F., Gwilliam, M. E., Peters, A. M., Myers, M. J., Gilbey, S. G., . . . Calam, J. (1993, 6). Amino acid infusion blocks renal tubular uptake of an indium-labelled somatostatin analogue. British journal of cancer, 67 (6), 1437-1439.
Heetfeld, M., Chougnet, C. N., Olsen, I. H., Rinke, A., Borbath, I., Crespo, G., . . . Knowledge Network members. (2015, 8). Characteristics and treatment of patients with G3 gastroenteropancreatic neuroendocrine neoplasms. Endocrine-related cancer, 22 (4), 657-664. doi: 10.1530/ERC-15-0119
Herder, W. W., Kwekkeboom, D. J., Feelders, R. A., Aken, M. O., Lamberts, S. W., Lely, A.-J., & Krenning, E. P. (2006). Somatostatin receptor imaging for neuroendocrine tumors. Pituitary, 9 (3), 243-248. doi: 10.1007/s11102-006-0270-5
Hope, T. A., Bergsland, E. K., Bozkurt, M. F., Graham, M., Heaney, A. P., Herrmann, K., Strosberg, J. R. (2018, 1). Appropriate Use Criteria for Somatostatin Receptor PET Imaging in Neuroendocrine Tumors. Journal of nuclear medicine: official publication, Society of Nuclear Medicine, 59 (1), 66-74. doi: 10.2967/jnumed. 117.202275
Howell, D. L., & O'Dorisio, M. S. (2012, 5). Management of neuroendocrine tumors in children, adolescents, and young adults. Journal of pediatric hematology/oncology, 34 Suppl 2, S64-S68. doi: 10.1097/MPH.0b013e31824e3885
Imam, H., Eriksson, B., Lukinius, A., Janson, E. T., Lindgren, P. G., Wilander, E., & Oberg, K. (1997). Induction of apoptosis in neuroendocrine tumors of the digestive system during treatment with somatostatin analogs. Acta oncologica (Stockholm, Sweden), 36 (6), 607-614.
Inzani, F., Petrone, G., & Rindi, G. (2018, 9). The New World Health Organization Classification for Pancreatic Neuroendocrine Neoplasia. Endocrinology and metabolism clinics of North America, 47 (3), 463-470. doi: 10.1016/j.ecl.2018.04.008
Inzani, F., Petrone, G., Fadda, G., & Rindi, G. (2017, 12). Cyto-histology in NET: what is necessary today and what is the future? Reviews in endocrine & metabolic disorders, 18 (4), 381-391. doi: 10.1007/s11154-017-9428-x
Jann, H., Roll, S., Couvelard, A., Hentic, O., Pavel, M., Müller-Nordhorn, J., . . . Pape, U.-F. (2011, 8). Neuroendocrine tumors of midgut and hindgut origin: tumor-node-metastasis classification determines clinical outcome. Cancer, 117 (15), 3332-3341. doi: 10.1002/cncr.25855
Joseph, S., Li, G., Lindholm, E., Zhou, Y., Go, V. L., Vinik, A. I., . . . Woltering, E. A. (2010, 10). A prospective trial on the effect of body mass index and sex on plasma octreotide levels in patients undergoing long-term octreotide LAR therapy. Pancreas, 39 (7), 964-966. doi: 10.1097/MPA.0b013e3181db01a8
Keck, K. J., Choi, A., Maxwell, J. E., Li, G., O'Dorisio, T. M., Breheny, P., . . . Howe, J. R. (2017, 8). Increased Grade in Neuroendocrine Tumor Metastases Negatively Impacts Survival. Annals of surgical oncology, 24 (8), 2206-2212. doi: 10.1245/s10434-017-5899-y
Kirshbom, P. M., Kherani, A. R., Onaitis, M. W., Feldman, J. M., & Tyler, D. S. (1998, 12). Carcinoids of unknown origin: comparative analysis with foregut, midgut, and hindgut carcinoids. Surgery, 124 (6), 1063-1070.
Klöppel, G. (2011, 10). Classification and pathology of gastroenteropancreatic neuroendocrine neoplasms. Endocrine-related cancer, 18 Suppl 1, S1-16. doi: 10.1530/ERC-11-0013
Kneifel, S., Cordier, D., Good, S., Ionescu, M. C., Ghaffari, A., Hofer, S., . . . Merlo, A. (2006, 6). Local targeting of malignant gliomas by the diffusible peptidic vector 1,4,7,10-tetraazacyclododecane-1-glutaric acid-4,7,10-triacetic acid-substance p. Clinical cancer research: an official journal of the American Association for Cancer Research, 12 (12), 3843-3850. doi: 10.1158/1078-0432.CCR-05-2820
Kulke, M. H., Shah, M. H., Benson, A. B., Bergsland, E., Berlin, J. D., Blaszkowsky, L. S., . . . National. (2015, 1). Neuroendocrine tumors, version 1.2015. Journal of the National Comprehensive Cancer Network: JNCCN, 13 (1), 78-108.
Kunz, P. L., Reidy-Lagunes, D., Anthony, L. B., Bertino, E. M., Brendtro, K., Chan, J. A., Society, N. A. (2013, 5). Consensus guidelines for the management and treatment of neuroendocrine tumors. Pancreas, 42 (4), 557-577. doi: 10.1097/MPA.0b013e31828e34a4
Kwekkeboom, D. J., & Krenning, E. P. (2016, 2). Peptide Receptor Radionuclide Therapy in the Treatment of Neuroendocrine Tumors. Hematology/oncology clinics of North America, 30 (1), 179-191. doi: 10.1016/j.hoc.2015.09.009
Kwekkeboom, D. J., Bakker, W. H., Kam, B. L., Teunissen, J. J., Kooij, P. P., Herder, W. W., . . . Krenning, E. P. (2003, 3). Treatment of patients with gastro-entero-pancreatic (GEP) tumours with the novel radiolabelled somatostatin analogue [¹⁷⁷Lu-DOTA(0), Tyr³] octreotate. European journal of nuclear medicine and molecular imaging, 30 (3), 417-422. doi: 10.1007/s00259-002-1050-8
Kwekkeboom, D. J., Bakker, W. H., Kooij, P. P., Konijnenberg, M. W., Srinivasan, A., Erion, J. L., . . . Krenning, E. P. (2001, 9). [177Lu-DOTAOTyr3] octreotate: comparison with [111In-DTP Ao] octreotide in patients. European journal of nuclear medicine, 28 (9), 1319-1325.
Kwekkeboom, D. J., Herder, W. W., Kam, B. L., Eijck, C. H., Essen, M., Kooij, P. P. Krenning, E. P. (2008, 5). Treatment with the radiolabeled somatostatin analog [177 Lu-DOTA 0,Tyr³] octreotate: toxicity, efficacy, and survival. Journal of clinical oncology: official journal of the American Society of Clinical Oncology, 26 (13), 2124-2130. doi: 10.1200/JCO.2007.15.2553
Kwekkeboom, D. J., Teunissen, J. J., Bakker, W. H., Kooij, P. P., Herder, W. W., Feelders, R. A., . . . Krenning, E. P. (2005, 4). Radiolabeled somatostatin analog [177Lu-DOTA0,Tyr³] octreotate in patients with endocrine gastroenteropancreatic tumors. Journal of clinical oncology: official journal of the American Society of Clinical Oncology, 23 (12), 2754-2762. doi: 10.1200/JCO.2005.08.066
Lau, D., Rutledge, C., & Aghi, M. K. (2015, 2). Cushing's disease: current medical therapies and molecular insights guiding future therapies. Neurosurgical focus, 38 (2), E11. doi: 10.3171/2014.10.FOCUS14700
Lau, S. C., Abdel-Rahman, O., & Cheung, W. Y. (2018, 8). Improved survival with higher doses of octreotide long-acting release in gastroenteropancreatic neuroendocrine tumors. Medical oncology (Northwood, London, England), 35 (9), 123. doi: 10.1007/s12032-018-1189-1
Ludlam, W. H., & Anthony, L. (2011, 10). Safety review: dose optimization of somatostatin analogs in patients with acromegaly and neuroendocrine tumors. Advances in therapy, 28 (10), 825-841. doi: 10.1007/s12325-011-0062-9
Massironi, S., Conte, D., & Rossi, R. E. (2016). Somatostatin analogues in functioning gastroenteropancreatic neuroendocrine tumours: literature review, clinical recommendations and schedules. Scandinavian journal of gastroenterology, 51 (5), 513-523. doi: 10.3109/00365521.2015.1115117
Meachem, S. J., Nieschlag, E., & Simoni, M. (2001, 11). Inhibin B in male reproduction: pathophysiology and clinical relevance. European journal of endocrinology, 145 (5), 561-571.
Modlin, I. M., Lye, K. D., & Kidd, M. (2003, 2). A 5-decade analysis of 13,715 carcinoid tumors. Cancer, 97 (4), 934-959. doi: 10.1002/cncr.11105
NICE guideline on Type 2 diabetes in adults: management, 2015. Published: 2 Dec. 2015. Last updated: 16 Dec. 2020. www.nice.org.uk/guidance/ng28
Oberg, K. (2012b, 7). Neuroendocrine tumors of the digestive tract: impact of new classifications and new agents on therapeutic approaches. Current opinion in oncology, 24 (4), 433-440. doi: 10.1097/CCO.0b013e328353d7ba
Oberg, K., Knigge, U., Kwekkeboom, D., Perren, A., & Group, E. S. (2012, 10). Neuroendocrine gastro-entero-pancreatic tumors: ESMO Clinical Practice Guidelines for diagnosis, treatment and follow-up. Annals of oncology: official journal of the European Society for Medical Oncology, 23 Suppl 7, vii124-vii130. doi: 10.1093/annonc/mds295
Pape, U.-F., Perren, A., Niederle, B., Gross, D., Gress, T., Costa, F., . . . B. C. (2012). ENETS Consensus Guidelines for the management of patients with neuroendocrine neoplasms from the jejuno-ileum and the appendix including goblet cell carcinomas. Neuroendocrinology, 95 (2), 135-156. doi: 10.1159/000335629
Pavel, M. E., Hainsworth, J. D., Baudin, E., Peeters, M., Horsch, D., Winkler, R. E., . . . Group, R. A.-2. (2011, 12). Everolimus plus octreotide long-acting repeatable for the treatment of advanced neuroendocrine tumours associated with carcinoid syndrome (RADIANT-2): a randomised, placebo-controlled, phase 3 study. Lancet (London, England), 378 (9808), 2005-2012. doi: 10.1016/S0140-6736 (11) 61742-X
Pavel, M., Baudin, E., Couvelard, A., Krenning, E., Öberg, K., Steinmüller, T., . . . B. C. (2012). ENETS Consensus Guidelines for the management of patients with liver and other distant metastases from neuroendocrine neoplasms of foregut, midgut, hindgut, and unknown primary. Neuroendocrinology, 95 (2), 157-176. doi: 10.1159/000335597
Pelosi, G., Rindi, G., Travis, W. D., & Papotti, M. (2014, 3). Ki-67 antigen in lung neuroendocrine tumors: unraveling a role in clinical practice. Journal of thoracic oncology: official publication of the International Association for the Study of Lung Cancer, 9 (3), 273-284. doi: 10.1097/JTO.0000000000000092
Perren, A., Couvelard, A., Scoazec, J.-Y., Costa, F., Borbath, I., Delle Fave, G., . . . A. C. (2017). ENETS Consensus Guidelines for the Standards of Care in Neuroendocrine Tumors: Pathology: Diagnosis and Prognostic Stratification. Neuroendocrinology, 105 (3), 196-200. doi: 10.1159/000457956
Pierik, F. H., Vreeburg, J. T., Stijnen, T., De Jong, F. H., & Weber, R. F. (1998, 9). Serum inhibin B as a marker of spermatogenesis. The Journal of clinical endocrinology and metabolism, 83 (9), 3110-3114. doi: 10.1210/jcem.83.9.5121
Reubi, J. C. (2003, 8). Peptide receptors as molecular targets for cancer diagnosis and therapy. Endocrine reviews, 24 (4), 389-427. doi: 10.1210/er.2002-0007
Reubi, J. C., Schär, J. C., Waser, B., Wenger, S., Heppeler, A., Schmitt, J. S., & Macke, H. R. (2000, 3). Affinity profiles for human somatostatin receptor subtypes SST1-SST5 of somatostatin radiotracers selected for scintigraphic and radiotherapeutic use. European journal of nuclear medicine, 27 (3), 273-282.
Reubi, J. C., Waser, B., Schaer, J. C., & Laissue, J. A. (2001, 7). Somatostatin receptor sst1-sst5 expression in normal and neoplastic human tissues using receptor autoradiography with subtype-selective ligands. European journal of nuclear medicine, 28 (7), 836-846.
Rindi, G. (2010). The ENETS guidelines: the new TNM classification system. Tumori, 96 (5), 806-809.
Rindi, G., Klersy, C., Albarello, L., Baudin, E., Bianchi, A., Buchler, M. W., . . . Falconi, M. (2018). Competitive Testing of the WHO 2010 versus the WHO 2017 Grading of Pancreatic Neuroendocrine Neoplasms: Data from a Large International Cohort Study. Neuroendocrinology, 107 (4), 375-386. doi: 10.1159/000494355
Rindi, G., Klimstra, D. S., Abedi-Ardekani, B., Asa, S. L., Bosman, F. T., Brambilla, E., Cree, I. A. (2018, 12). A common classification framework for neuroendocrine neoplasms: an International Agency for Research on Cancer (IARC) and World Health Organization (WHO) expert consensus proposal. Modern pathology: an official journal of the United States and Canadian Academy of Pathology, Inc, 31 (12), 1770-1786. doi: 10.1038/s41379-018-0110-y
Rindi, G., Petrone, G., & Inzani, F. (2014, 6). The 2010 WHO classification of digestive neuroendocrine neoplasms: a critical appraisal four years after its introduction. Endocrine pathology, 25 (2), 186-192. doi: 10.1007/s12022-014-9313-z
Rinke, A., Müller, H.-H., Schade-Brittinger, C., Klose, K.-J., Barth, P., Wied, M., . . . Group, P. R. (2009, 10). Placebo-controlled, double-blind, prospective, randomized study on the effect of octreotide LAR in the control of tumor growth in patients with metastatic neuroendocrine midgut tumors: a report from the PROMID Study Group. Journal of clinical oncology: official journal of the American Society of Clinical Oncology, 27 (28), 4656-4663. doi: 10.1200/JCO.2009.22.8510
Rolleman, E. J., Valkema, R., Jong, M., Kooij, P. P., & Krenning, E. P. (2003, 1). Safe and effective inhibition of renal uptake of radiolabelled octreotide by a combination of lysine and arginine. European journal of nuclear medicine and molecular imaging, 30(1), 9-15. doi: 10.1007/s00259-002-0982-3
Sackstein, P. E., O'Neil, D. S., Neugut, A. I., Chabot, J., & Fojo, T. (2018, 8). Epidemiologic trends in neuroendocrine tumors: An examination of incidence rates and survival of specific patient subgroups over the past 20 years. Seminars in oncology, 45 (4), 249-258. doi: 10.1053/j.seminoncol.2018.07.001
Sierra, M. L., Agazzi, A., Bodei, L., Pacifici, M., Aricò, D., De Cicco, C., . . . Paganelli, G. (2009, 12). Lymphocytic toxicity in patients after peptide-receptor radionuclide therapy (PRRT) with ¹⁷⁷Lu-DOTATATE and 90Y-DOTATOC. Cancer biotherapy & radiopharmaceuticals, 24 (6), 659-665. doi: 10.1089/cbr.2009.0641
Singh, S., Carnaghi, C., Buzzoni, R., Pommier, R. F., Raderer, M., Tomasek, J., . . . RAD001 in Advanced Neuroendocrine Tumors, F. T.-4. (2018). Everolimus in Neuroendocrine Tumors of the Gastrointestinal Tract and Unknown Primary. Neuroendocrinology, 106 (3), 211-220. doi: 10.1159/000477585
Sorbye, H., Baudin, E., Borbath, I., Caplin, M., Chen, J., Cwikla, J. B., . . . ENETS 2016 Munich Advisory Board Participants. (2019). Unmet Needs in High-Grade Gastroenteropancreatic Neuroendocrine Neoplasms (WHO G3). Neuroendocrinology, 108 (1), 54-62. doi: 10.1159/000493318
Strosberg, J., El-Haddad, G., Wolin, E., Hendifar, A., Yao, J., Chasen, B., . . . Investigators, N. E.-1. (2017, 1). Phase 3 Trial of, javax.xml.bind.JAXBElement@11e0f286, Lu-Dotatate for Midgut Neuroendocrine Tumors. The New England journal of medicine, 376 (2), 125-135. doi: 10.1056/NEJMoal607427
Strosberg, J., Wolin, E., Chasen, B., Kulke, M., Bushnell, D., Caplin, M., . . . Group, N. E.-1. (2018, 9). Health-Related Quality of Life in Patients With Progressive Midgut Neuroendocrine Tumors Treated With, javax.xml.bind.JAXBElement@52bac87, Lu-Dotatate in the Phase III NETTER-1 Trial. Journal of clinical oncology: official journal of the American Society of Clinical Oncology, 36 (25), 2578-2584. doi: 10.1200/JCO.2018.78.5865
Strosberg J, Leeuwenkamp O, Siddiqui MK (2021) Peptide receptor radiotherapy re-treatment in patients with progressive neuroendocrine tumors: A systematic review and meta-analysis. Cancer Treat Rev; 93:102141.
Teunissen, J. J., Krenning, E. P., Jong, F. H., Rijke, Y. B., Feelders, R. A., Aken, M. O., . . . Kwekkeboom, D. J. (2009, 11). Effects of therapy with [¹⁷⁷Lu-DOTA 0, Tyr 3] octreotate on endocrine function. European journal of nuclear medicine and molecular imaging, 36 (11), 1758-1766. doi: 10.1007/s00259-009-1151-8
Uri, I., & Grozinsky-Glasberg, S. (2018). Current treatment strategies for patients with advanced gastroenteropancreatic neuroendocrine tumors (GEP-NETs). Clinical diabetes and endocrinology, 4, 16. doi: 10.1186/s40842-018-0066-3
van der Zwan, W. A., Brabander, T., Kam, B. L., Teunissen, J. J., Feelders, R. A., Hofland, J., . . . Herder, W. W. (2019, 3). Salvage peptide receptor radionuclide therapy with [177Lu-DOTA,Tyr³] octreotate in patients with bronchial and gastroenteropancreatic neuroendocrine tumours. European journal of nuclear medicine and molecular imaging, 46 (3), 704-717. doi: 10.1007/s00259-018-4158-1
Vélayoudom-Céphise, F.-L., Duvillard, P., Foucan, L., Hadoux, J., Chougnet, C. N., Leboulleux, S., . . . Baudin, E. (2013, 10). Are G3 ENETS neuroendocrine neoplasms heterogeneous? Endocrine-related cancer, 20 (5), 649-657. doi: 10.1530/ERC-13-0027
Welin, S. V., Janson, E. T., Sundin, A., Stridsberg, M., Lavenius, E., Granberg, D., . . . Eriksson, B. K. (2004, 7). High-dose treatment with a long-acting somatostatin analogue in patients with advanced midgut carcinoid tumours. European journal of endocrinology, 151 (1), 107-112.
Wolchok, J. D., Hoos, A., O'Day, S., Weber, J. S., Hamid, O., Lebbé, C., . . . Hodi, F. S. (2009, 12). Guidelines for the evaluation of immune therapy activity in solid tumors: immune-related response criteria. Clinical cancer research: an official journal of the American Association for Cancer Research, 15 (23), 7412-7420. doi: 10.1158/1078-0432.CCR-09-1624
Wolin, E. M. (2012, 9). The expanding role of somatostatin analogs in the management of neuroendocrine tumors. Gastrointestinal cancer research: GCR, 5 (5), 161-168.
Yao, J. C., Fazio, N., Singh, S., Buzzoni, R., Carnaghi, C., Wolin, E., . . . RAD001 in Advanced Neuroendocrine Tumours, F. T.-4. (2016, 3). Everolimus for the treatment of advanced, non-functional neuroendocrine tumours of the lung or gastrointestinal tract (RADIANT-4): a randomised, placebo-controlled, phase 3 study. Lancet (London, England), 387 (10022), 968-977. doi: 10.1016/S0140-6736 (15) 00817-X
Yao, J. C., Hassan, M., Phan, A., Dagohoy, C., Leary, C., Mares, J. E., . . . Evans, D. B. (2008, 6). One hundred years after “carcinoid”: epidemiology of and prognostic factors for neuroendocrine tumors in 35,825 cases in the United States. Journal of clinical oncology: official journal of the American Society of Clinical Oncology, 26 (18), 3063-3072. doi: 10.1200/JCO.2007.15.4377
Yao, J. C., Lombard-Bohas, C., Baudin, E., Kvols, L. K., Rougier, P., Ruszniewski, P., . . . Wiedenmann, B. (2010, 1). Daily oral everolimus activity in patients with metastatic pancreatic neuroendocrine tumors after failure of cytotoxic chemotherapy: a phase II trial. Journal of clinical oncology: official journal of the American Society of Clinical Oncology, 28 (1), 69-76. doi: 10.1200/JCO.2009.24.2669
Yao, J. C., Shah, M. H., Ito, T., Bohas, C. L., Wolin, E. M., Van Cutsem, E., . . . RAD001 in Advanced Neuroendocrine Tumors, T. T.-3. (2011, 2). Everolimus for advanced pancreatic neuroendocrine tumors. The New England journal of medicine, 364 (6), 514-523. doi: 10.1056/NEJMoa1009290
Zatelli, M. C., Guadagno, E., Messina, E., Lo Calzo, F., Faggiano, A., Colao, A., & Group, N. I. (2018, 6). Open issues on G3 neuroendocrine neoplasms: back to the future. Endocrine-related cancer, 25 (6), R375-R384. doi: 10.1530/ERC-17-0507

### EXAMPLES

Hereinafter, the present disclosure is described in more details and specifically with reference to the examples, which however are not intended to limit the present disclosure.

### Example 1a: Clinical Trial Protocol

A phase III multi-center, randomized, open-label study to evaluate the efficacy and safety of Lutathera in patients with Grade 2 and Grade 3 advanced GEP-NET

| Protocol | |
|---|---|
| number | CAAA601A22301 |
| Full Title | This is a multicenter, stratified, randomized, open-label comparator- |
| | controlled, Phase III study in patients with somatostatin receptor positive, |
| | well-differentiated G2 and G3, advanced GEP NETs, diagnosed within 6 |
| | months prior to screening, comparing treatment with Lutathera (7.4 |
| | GBq/200 mCi × 4 administrations every 8 ± 1 weeks; cumulative dose: |
| | 29.6 GBq/800mCi) plus octreotide long-acting (30 mg every 8 weeks |
| | during Lutathera treatment and every 4 weeks after last Lutathera |
| | treatment) and high dose octreotide long-acting (60 mg every 4 weeks). |
| Brief title | A Phase III multi-center, randomized, open-label study to evaluate the |
| | efficacy and safety of Lutathera in patients with Grade 2 and Grade 3 |
| | advanced GEP-NET. |
| Sponsor and | Sponsored by Advanced Accelerator Applications, Phase III Study. |
| Clinical Phase | |
| Purpose and | The pivotal Phase III NETTER-1 study showed that Lutathera with best |
| rationale | supportive care (30 mg octreotide long-acting) provided a significant |
| | increase in PFS to patients with progressive midgut carcinoid tumors (at |
| | enrollment) compared to patients treated with high dose (60 mg) |
| | octreotide long-acting. The NETTER-1 patient population included 34.5% |
| | of patients with G2 NET (65.5% G1), while G3 NETs were excluded. Only |
| | patients progressive on SSAs were eligible (2^{nd} line), SSA-naïve patients |
| | were excluded. |
| | The aim of the NETTER-2 study is to determine if Lutathera in |
| | combination with octreotide long-acting prolongs PFS in GEP-NET |
| | patients with high proliferation rate tumors (G2 and G3), when given as a |
| | 1^{st} line treatment in comparison to treatment with high dose (60 mg) |
| | octreotide long-acting. SSA-naïve patients are eligible, as well as patients |
| | previously treated with SSAs in the absence of progression. |
| | Based on extensive experience with Lutathera as well as octreotide LAR |
| | in adult GEP NET patients, and the relevance of the molecular target in |
| | adolescent GEP NET patients, the study will be open to adolescents |
| | aged ≥15 years and >40 kg body weight (BW); younger patients are not |
| | expected to present with the disease meeting the severity criteria for this |
| | trial. |
| Primary | To demonstrate that Lutathera is superior to active comparator in |
| Objective(s) | delaying the time-to-first occurrence of progression or death (PFS) as first |
| | line treatment. |
| Key Secondary | To demonstrate the superiority of Lutathera, compared to active |
| Objectives | comparator, in terms of objective response |
| | To demonstrate the superiority of Lutathera, compared to active |
| | comparator, in terms of time to deterioration in selected QoL items/scales. |
| Other | To evaluate the efficacy of Lutathera, compared to active comparator, in |
| Secondary | keeping the disease under control |
| Objectives | To evaluate the efficacy of Lutathera, compared to active comparator, in |
| | terms of duration of response |
| | To evaluate the safety and tolerability of Lutathera |
| | To evaluate the effect of Lutathera on overall survival |
| Study design | Overall, 222 patients will be randomized (2:1 randomization ratio) to |
| | receive treatment with Lutathera (7.4GBq/200 mCi × 4 administrations |
| | every 8 ± 1 weeks; cumulative dose: 29.6 GBq/800mCi) plus octreotide |
| | long-acting (30 mg every 8 weeks during Lutathera treatment and every |
| | 4 weeks after last Lutathera treatment) or high dose octreotide long- |
| | acting (60 mg every 4 weeks). Randomization will be stratified by Grade |
| | (G2 vs G3) and tumor origin (pNET vs other origin). |
| | The primary endpoint of the study is PFS centrally assessed (target |
| | HR = 0.5; 90% power, 1-sided α = 2.5%). The primary analysis will be |
| | performed after 99 PFS events (99 evaluable and centrally confirmed |
| | disease progressions or death events) have occurred. |
| | The study consists of a Screening Phase, a Treatment Phase, an |
| | optional Treatment Extension Phase (cross-over), an optional Re- |
| | treatment Phase and a Follow up Phase. |
| | Screening Phase |
| | The screening phase must be shortened as much as possible, in order |
| | to treat the patients possibly within 2 weeks after the consent signature. |
| | The randomization must be performed immediately after all eligibility |
| | criteria are verified. As Lutathera production and shipment will take |
| | circa 12 days to be arranged, if a patient is randomized in the Lutathera |
| | arm, Lutathera first dose must be ordered immediately after |
| | randomization. |
| | The baseline CT/MRI scan should be taken possibly on the same day |
| | as randomization or immediately before (within 1 week) to ensure that it |
| | reflects the disease status closely before the therapy start. |
| | Treatment Phase |
| | During the Treatment Phase, objective tumor response will be assessed |
| | at W16 ± 1, W24 ± 1 and then every 12 ± 1 weeks from the randomization |
| | date, according to RECIST 1.1 criteria (central + local assessment up to |
| | first progression, then only local assessment). |
| | Duration of the Treatment Phase: |
| | Before the PFS primary analysis (i.e. 99 evaluable and centrally |
| | confirmed disease progressions or death events), patients continue the |
| | Treatment Phase until progression; after the PFS primary analysis, the |
| | Treatment Phase duration is limited to 72 weeks. |
| | At any time during the study (before or after the PFS primary analysis) |
| | any progressive patient (based on central imaging assessment) |
| | immediately ceases the Treatment Phase and proceeds to the Follow-up |
| | Phase. Patients who experience disease progression in the Lutathera |
| | arm after having received and benefitted from 4 doses/cycles as initial |
| | treatment have the option, if eligible, to enroll for re-treatment with |
| | Lutathera. In addition, patients randomized in the control arm have the |
| | option, if eligible, to enroll for post-progression cross-over with Lutathera. |
| | Optional Treatment Extension Phase (cross-over) |
| | In the control arm, any patient with radiological progression according to |
| | RECIST (based on central assessment) has the option to enroll for post- |
| | progression cross-over, upon signature of a new informed consent, to |
| | receive maximum 4 cycles of Lutathera (7.4 GBq/200 mCi × 4 cycles; |
| | cumulative dose: 29.6 GBq/800mCi) plus 30 mg octreotide long acting |
| | every 8 weeks. If RECIST progression occurs after Week 72 post the |
| | primary end point analysis, the decision to enroll the patient in the |
| | cross-over phase will be based on local assessment. The time window |
| | to start Lutathera during the cross-over phase is within 4 years after the |
| | last patient has been randomized. |
| | Optional Re-treatment Phase |
| | Patients in the Lutathera arm with radiological progression based on |
| | RECIST criteria in central assessment will be offered enrollment in the |
| | optional re-treatment phase upon signature of a new informed consent. |
| | If RECIST progression occurs after Week 72 post the primary end point |
| | analysis, the decision to enroll the patient in re-treatment will be based |
| | on local assessment. |
| | The criteria to enter the re-treatment phase include achievement of |
| | stabilization of disease or radiological response to Lutathera initial |
| | treatment for at least 6 months after receiving the 4^{th} Lutathera dose |
| | and good safety/tolerability. If a patient's response has changed from |
| | PR/CR to SD within 6 months after the 4^{th} Lutathera dose, the patient is |
| | still eligible for re-treatment, provided there is no documented |
| | progression within 6 months. Patients who received other systemic |
| | treatments for GEP-NET after progression (except somatostatin |
| | analogues) are not eligible for re-treatment. The time window to start re- |
| | treatment in this study is within 4 years after the last patient has been |
| | randomized. |
| | In the re-treatment phase, patients will initially receive 2 administrations |
| | of Lutathera 7.4 GBq/200 mCi at 8-week interval. Based on the |
| | physician's judgment of the clinical benefit derived from the first 2 |
| | doses, up to 2 additional doses of Lutathera may be administered. A |
| | maximum of 4 cycles of Lutathera is allowed during the re-treatment |
| | period. All safety and efficacy assessments in the re-treatment period |
| | will be performed locally (SRI images must also be submitted to the |
| | central images reading center possibly within 1 month) and following the |
| | schedule in the initial treatment period. Assessments will be continued |
| | until disease progression is documented by investigator or until End of |
| | Study, whichever occurs first. |
| | Follow up Phase |
| | At the end of the Treatment Phase or after discontinuation for any cause |
| | (including disease progression), all patients will continue to be followed |
| | up to 3 years to continue data collection for the secondary endpoints of |
| | the study, such as long term safety and overall survival. Patients |
| | included in the optional crossover or re-treatment phase will be followed |
| | up at least 6 months and up to 3 years (or until EoS, whatever comes |
| | first). |
| | During the Follow-up Phase, serious adverse events and adverse events of |
| | special interest (AESI) related to the study treatment as well as AESI of |
| | secondary hematological malignancies irrespective of causality, will be |
| | reported. Anti-tumor treatments administered after |
| | progression/discontinuation, disease status based on local CT/MRI |
| | assessment, and OS data will be collected every 6 months in both arms. |
| | End of the Study |
| | The End of Study is after 4 years have elapsed from the randomization |
| | of the last patient or 6 months after the last cross-over or re-treatment |
| | dose in the study, whichever occurs last. The time window to start |
| | cross-over or re-treatment with Lutathera in this study is within 4 years |
| | after the last patient has been randomized. For patients in the Lutathera |
| | arm who progress beyond this window, access to re-treatment with |
| | Lutathera may be granted via Post Study Drug Supply (PSDS) |
| | programs. |
| Population | In this study, safety and efficacy of treatment with Lutathera plus |
| | octreotide long-acting (30 mg) versus high dose octreotide long-acting (60 |
| | mg) is evaluated in patients ≥15 years with somatostatin receptor positive, |
| | well differentiated G2 (Ki67 index ≥10% to ≤20%) and G3 (Ki67 >20% |
| | and ≤55%) advanced GEP-NETs. Patients with documented RECIST |
| | progression to previous treatments for the current GEP-NET at any time |
| | prior to randomization are not eligible to participate in this study. |
| Key Inclusion | Subjects eligible for inclusion in this study must meet all of the following |
| criteria | criteria: |
| | 1. Presence of metastasized or locally advanced, inoperable (curative |
| | intent) histologically proven, well differentiated Grade 2 or Grade 3 |
| | gastroenteropancreatic neuroendocrine (GEP-NET) tumor diagnosed |
| | within 6 months prior to screening. |
| | 2. Ki67 index ≥10 and ≤55%. |
| | 3. Patients ≥15 years of age and a body weight of >40 kg at screening. |
| | 4. Expression of somatostatin receptors on all target lesions |
| | documented by CT/MRI scans, assessed by any of the following |
| | somatostatin receptor imaging (SRI) modalities within 3 months prior |
| | to randomization: [68Ga]-DOTA-TOC (e.g. Somakit-TOC ®) PET/CT |
| | imaging (or MRI when applicable based on target lesions), [68Ga]- |
| | DOTA-TATE PET/CT (or MRI when applicable based on target |
| | lesions) imaging (e.g. NETSPOT ®), Somatostatin Receptor |
| | scintigraphy (SRS) with [111In]-pentetreotide (Octreoscan ® |
| | SPECT/CT), SRS with [99mTc]-Tektrotyd, [64Cu]-DOTA-TATE |
| | PET/CT imaging (or MRI when applicable based on target lesions). |
| | 5. The tumor uptake observed in the target lesions must be > normal |
| | liver uptake. |
| | 6. Karnofsky Performance Score (KPS) ≥60. |
| | 7. Presence of at least 1 measurable site of disease. |
| | 8. Patients who have provided a signed informed consent form to |
| | participate in the study, obtained prior to the start of any protocol |
| | related activities. |
| Key Exclusion | Subjects meeting any of the following criteria are not eligible for inclusion |
| criteria | in this study. |
| | 1. Creatinine clearance <40 mL/min calculated by the Cockroft Gault |
| | method. |
| | 2. Hb concentration <5.0 mmol/L (<8.0 g/dL); WBC <2 × 10⁹/L |
| | (2000/mm³); platelets <75 × 10⁹/L (75 × 10³/mm³). |
| | 3. Total bilirubin >3 × ULN. |
| | 4. Serum albumin <3.0 g/dL unless prothrombin time is within the normal |
| | range. |
| | 5. Pregnancy or lactation. |
| | 6. A) Women of child-bearing potential, defined as all women |
| | physiologically capable of becoming pregnant, are not allowed to |
| | participate in this study UNLESS they are using highly effective |
| | methods of contraception throughout the study treatment period |
| | (including cross-over and re-treatment, if applicable) and for 7 months |
| | after study drug discontinuation. |
| | B) Sexually active male patients, unless they agree to remain |
| | abstinent (refrain from heterosexual intercourse) or be willing to use |
| | condoms and highly effective methods of contraception with female |
| | partners of childbearing potential or pregnant female partners during |
| | the treatment period (including cross-over and re-treatment, if |
| | applicable) and for 4 months after study drug discontinuation. In |
| | addition, male patients must refrain from donating sperm during this |
| | same period. |
| | 7. Peptide receptor radionuclide therapy (PRRT) at any time prior to |
| | randomization in the study. |
| | 8. Documented RECIST progression to previous treatments for the |
| | current GEP-NET at any time prior to randomization. |
| | 9. Patients for whom in the opinion of the investigator other therapeutic |
| | options (eg chemo-, targeted therapy) are considered more |
| | appropriate than the therapy offered in the study, based on patient |
| | and disease characteristics. |
| | 10. Any previous therapy with Interferons, Everolimus (mTOR-inhibitors), |
| | chemotherapy or other systemic therapies of GEP-NET administered |
| | for more than 1 month or within 12 weeks prior to randomization in |
| | the study. |
| | 11. Any previous radioembolization, chemoembolization and |
| | radiofrequency ablation for GEP-NET. |
| | 12. Any surgery within 12 weeks prior to randomization in the study. |
| | 13. Known brain metastases, unless these metastases have been treated |
| | and stabilized for at least 24 weeks, prior to screening in the study. |
| | Patients with a history of brain metastases must have a head CT or |
| | MRI with contrast to document stable disease prior to randomization |
| | in the study. |
| | 14. Uncontrolled congestive heart failure (NYHA II, III, IV). Patients with |
| | history of congestive heart failure who do not violate this exclusion |
| | criterion will undergo an evaluation of their cardiac ejection fraction |
| | prior to randomization via echocardiography. The results from an |
| | earlier assessment (not exceeding 30 days prior to randomization) |
| | may substitute the evaluation at the discretion of the Investigator, if |
| | no clinical worsening is noted. The patient's measured cardiac |
| | ejection fraction in these patients must be ≥40% before |
| | randomization. |
| | 15. QTcF >470 msec for females and QTcF >450 msec for males or |
| | congenital long QT syndrome. |
| | 16. Uncontrolled diabetes mellitus as defined by hemoglobin A1c |
| | value >7.5%. |
| | 17. Hyperkaleamia >6.0 mmol/L (CTCAE Grade 3) which is not corrected |
| | prior to study enrolment. |
| | 18. Any patient receiving treatment with short-acting octreotide, which |
| | cannot be interrupted for 24 h before and 24 h after the administration |
| | of Lutathera, or any patient receiving treatment with SSAs (eg |
| | octreotide long-acting), which cannot be interrupted for at least 6 |
| | weeks before the administration of Lutathera. |
| | 19. Patients with any other significant medical, psychiatric, or surgical |
| | condition, currently uncontrolled by treatment, which may interfere |
| | with the completion of the study. |
| | 20. Prior external beam radiation therapy to more than 25% of the bone |
| | marrow. |
| | 21. Current spontaneous urinary incontinence. |
| | 22. Other known co-existing malignancies except non-melanoma skin |
| | cancer and carcinoma in situ of the uterine cervix, unless definitively |
| | treated and proven no evidence of recurrence for 5 years. |
| | 23. Patient with known incompatibility to CT Scans with I.V. contrast due |
| | to allergic reaction or renal insufficiency. If such a patient can be |
| | imaged with MRI, then the patient would not be excluded. |
| | 24. Hypersensitivity to any somatostatin analogues, to the IMPs active |
| | substance or to any of the excipients. |
| | 25. Patients who have participated in any therapeutic clinical |
| | study/received any investigational agent within the last 30 days. |
| Study treatment | In this study, approximately 222 patients with advanced G2-3 GEP-NET |
| | will be randomized (2:1 randomization ratio) to receive treatment with |
| | Lutathera (7.4 GBq or 200 mCi × 4 administrations every 8 ± 1 weeks; |
| | cumulative dose: 29.6 GBq or 800 mCi) plus octreotide long-acting |
| | standard dose (30 mg every 8 weeks during Lutathera treatment and |
| | every 4 weeks after last Lutathera treatment) or octreotide long-acting |
| | high dose (60 mg every 4 weeks). |
| | The investigational drug product Lutathera ® (¹⁷⁷Lu-DOTA⁰-Tyr³- |
| | Octreotate) will be provided by the Sponsor. The Sponsor will also |
| | provide the 2.5% Lys-Arg sterile amino acid solution for infusion (if it |
| | can't be compounded at the hospital Pharmacy), as well as octreotide |
| | long-acting (Sandostatin ® LAR Depot) for the entire duration of the |
| | Treatment Phase (and optional Treatment Extension Phase in case of |
| | cross-over) of the study. During the re-treatment period, Lutathera ® and |
| | 2.5% Lys-Arg sterile amino acid solution for infusion (if it can't be |
| | compounded at the hospital Pharmacy) will be provided by the Sponsor. |
| | Patients will switch to prescribed drugs in the follow up phase. |
| | Anti-emetics, SRI imaging agents, short-acting octreotide or any other |
| | supportive care medication will not be supplied by the Sponsor. |
| Efficacy | During the Treatment Phase, objective tumor response will be assessed |
| assessments | at W16 ± 1, W24 ± 1 and then every 12 ± 1 weeks from the randomization |
| | date, according to RECIST 1.1 criteria (central + local assessment up to |
| | first progression, then only local assessment). During the Follow up |
| | Phase RECIST objective tumor response will be assessed locally |
| | (scans every 6 months). |
| Pharmacokinetic | Patients in the control arm (all countries except for China) will yield PK |
| assessments | data from blood sampling performed at pre-dose with respect to the |
| | octreotide long-acting i.m. injection for the 2nd, 4th, 5th and 7th |
| | treatment cycle (that is respectively at week 4, 12, 16 and 24) for the |
| | determination of plasma trough levels. Data will be processed using a |
| | population based model, for exploratory purposes. |
| Key safety | All adverse events (AEs), whether or not spontaneously reported by the |
| assessments | patient, will be recorded starting from the signing of the ICF until the end |
| | of the Treatment Phase/optional Treatment Extension Phase in case of |
| | cross-over/optional re-treatment. During the Follow up Phase only |
| | related serious adverse events and related AESI other than secondary |
| | hematological malignancies will be recorded. AESI of secondary |
| | hematological malignancies will be recorded during the whole study |
| | irrespective of causality. |
| Data analysis | The primary efficacy and safety analyses will be performed after |
| | observing approximately 99 PFS events. At that time a soft database |
| | lock will ensure the integrity of the data and make further data entry for |
| | the continuation of the trial possible. |
| | The primary endpoint of the study is progression-free survival (PFS), |
| | defined as the time from the date of randomization to the date of the first |
| | documented progression or death due to any cause. PFS will be |
| | assessed via central review according to RECIST 1.1. |
| | The following statistical hypotheses will be tested to address the primary |
| | efficacy objective: |
| | H₀₁ (null hypotheses): Θ₁ ≥ 0 vs. Hₐ₁ (alternative hypotheses): Θ₁ < 0 |
| | Where Θ₁ is the log hazard ratio of PFS in the Lutathera plus Standard |
| | Dose octreotide long-acting (30 mg) (investigational) arm vs. High Dose |
| | octreotide long-acting (60 mg) (control) arm. |
| | The primary efficacy analysis to test this hypothesis and compare the two |
| | treatment groups will consist of a stratified log-rank test at an overall one- |
| | sided 2.5% level of significance in favor of the Lutathera plus Standard |
| | Dose octreotide long-acting (30 mg) arm. The stratification will be based |
| | on the following randomization stratification factors (grade: G2 vs. G3; |
| | and tumor origin: pNET vs other origin). |
| | Secondary objectives of the study are to compare the two treatment |
| | groups with respect to objective response rate, time to deterioration in |
| | selected EORTC QLQ-C30 and QLQ-G.I.NET21 QOL items/scales, |
| | disease control rate, duration of response, safety and overall survival. |
| | Objective response rate and time to deterioration in EORTC QLQ-C30 |
| | global health status, diarrhea, fatigue, and pain are identified as key |
| | secondary endpoints. |
| | A hierarchical testing procedure will be used to control the overall type I |
| | error in assessing the primary and key secondary objectives. |

### Example 1b: First Interpretable Results (FIR), PFS Primary Analysis

### Summary of the Results

| | | |
|---|---|---|
| Key parameter | Definition | Outcome |

Primary endpoint

| | | |
|---|---|---|
| PFS | Time from randomization | The study did meet the primary |
| | to the first line | objective for PFS. |
| | progression (centrally | stratified HR (95% Cl): 0.276 (0.182, 0.418) |
| | assessed according to | One-sided stratified p-value: <0.0001 |
| | RECIST 1.1) or death | Median PFS in months (95% Cl): |
| | due to any cause | 22.8 (19.4, NE) vs. 8.5 (7.7, 13.8) for |
| | | Lutathera vs. high-dose Octreotide arms |

Key Secondary Endpoint(s)

| | | |
|---|---|---|
| ORR | Rate of participants with | The study did meet the key secondary |
| | best overall response of | objective for ORR. |
| | unconfirmed partial | ORR (95% Cl): 43.0% (35.0%, 51.3%) vs. |
| | response (PR) or | 9.3% (3.8%, 18.3%) for Lutathera vs. high-dose |
| | unconfirmed complete | Octreotide arms |
| | response (CR) (centrally | Stratified Odds Ratio: 7.81 (3.32, 18.40) |
| | assessed according to | Stratified p-value: <0.0001 |
| | RECIST 1.1) | |

Other secondary endpoint(s)

| | | The most frequent AEs: | | |
|---|---|---|---|---|
| | | Preferred | Lutathera | Octreotide |
| Safety | Incidence of AEs | Term | (%) | LAR (%) |
| | | Nausea | 40 (27.2) | 13 (17.8) |
| | | Diarrhoea | 38 (25.9) | 25 (34.2) |
| | | Abdominal pain | 26 (17.7) | 20 (27.4) |
| | | The most frequent SAE: | | |
| | | Preferred | Lutathera | Octreotide |
| Safety | Incidence of SAEs | Term | (%) | LAR (%) |
| | | Small intestinal | 5 (3.4) | 0 |
| | | obstruction | | |

This study was a randomized, open-label, active comparator, multicenter, phase III study in participants with newly diagnosed Grade 2 and Grade 3 advanced GEP-NET receiving either Lutathera plus long-acting repeatable (LAR) Octreotide (hereafter referred to as Lutathera) or High-dose Octreotide LAR (hereafter referred to as High-dose Octreotide). The randomization was stratified by Tumor Grade (Grade 2 or Grade 3) and Tumor Origin (pNET or Other).
This document describes the first interpretable results (FIR) of the PFS primary analysis when 99 PFS events have been documented per central review. The data cut-off date for this analysis was 20 Jul. 2023 and the study is ongoing. The median (min, max) duration of follow-up was 23.2 months (9.2-41.9) (from randomization to data cut-off date). Key efficacy and safety results are presented without any clinical interpretation.
A total of 226 participants (Full Analysis Set) were randomized with a 2:1 ratio to Lutathera (n=151) or High-dose Octreotide (n=75) between 22 Jan. 2020 and 13 Oct. 2022. There were 220 participants in the Safety Set, 147 participants in the Lutathera arm and 73 participants in the High-dose Octreotide arm.
Primary PFS endpoint and Key secondary endpoints were analyzed in a hierarchical fashion to protect the type I error rate. The order of the hypothesis testing is PFS, then ORR.
Treatment discontinuations in randomized period were reported for 48.3% of participants in the Lutathera arm and 80.0% of participants in the High-dose Octreotide arm. The most frequently reported reason for discontinuation was progressive disease (27.8% in the Lutathera arm and 58.7% in the High-dose Octreotide arm). Treatment discontinuation due to adverse event was 5.3% in the Lutathera arm and 1.3% in the High-dose Octreotide arm.
29 (38.7%) participants randomized to the High-dose Octreotide crossed over to receive Lutathera after disease progression. 8 (5.3%) participants randomized to Lutathera arm received a second course of treatment with Lutathera (re-treatment) after disease progression.
In the randomized treatment period, the median duration of exposure to Lutathera was 32 weeks. 87.8% of participants received the full course of 4 Lutathera doses. The median duration of exposure to Octreotide in the Lutathera arm was 71.0 weeks compared to 40.3 weeks in the High-dose Octreotide arm.
The study did meet its primary objective. The primary endpoint, progression-free survival (PFS) based on central radiology review, was statistically significantly different between the treatment arms (stratified Log-rank test p<0.0001, one-sided) with an estimated 72% risk reduction in the Lutathera arm compared to the High-dose Octreotide arm (Hazard ratio: 0.276 with 95% CI: (0.182, 0.418)). The median PFS times (95% CI) are 22.8 months (19.4, NE) and 8.5 months (7.7, 13.8), respectively.
The first key secondary endpoint tested in a hierarchical fashion was ORR. It was statistically significant with the stratified odds ratio of 7.81 and a stratified one-sided p-value <0.0001. The ORR based on central assessment was 43.0% (95% CI: 35.0, 51.3) in the Lutathera arm and 9.3% (95% CI: 3.8, 18.3) in the High-dose Octreotide arm.
The following adverse events (AEs) were reported in the Lutathera and the High-dose Octreotide arms during the randomized treatment period, respectively:

| | |
|---|---|
| Any AEs: | 92.5% vs. 94.5% |
| Grade ≥3 AEs: | 35.4% vs. 27.4% |
| Treatment-related: | 15.6% vs. 4.1% |
| Serious AEs: | 20.4% vs. 20.5% |
| Treatment-related: | 5.4% vs. 1.4% |
| AEs leading to discontinuation of | 4.8% vs. 2.7% |
| Lutathera and/or Octreotide | |
| Lutathera | 2.0% vs. NA |
| Octreotide | 3.4% vs. 2.7% |

The most common adverse events (>20% in either arm) during the randomized treatment period by preferred term, were nausea (27.2% vs. 17.8%), diarrhoea (25.9% vs. 34.2%) and abdominal pain (17.7% vs. 27.4%) on the Lutathera and the High-dose Octreotide arms, respectively.
The most common Grade 3/4 AEs (≥3% in either arm) during randomized treatment period by preferred term were Lymphocyte count decreased (5.4% vs. 0%), GGT increased (4.8% vs. 2.7%), small intestinal obstruction (3.4% vs. 0%) and abdominal pain (2.7% vs. 4.1%) on the Lutathera and the High-dose Octreotide arms, respectively.
A total of 32 (21.8%) and 14 (19.2%) subjects died during the study in the Lutathera and the High-dose Octreotide arms respectively, with 2 (1.4%) and 4 (5.5%) within 30 days after the last dose in the randomized treatment period. The main causes of deaths during the study on the Lutathera and the High-dose Octreotide arms, respectively, were disease under study (20.4% vs. 17.8%) and adverse event (1.4% vs. 1.4%). All deaths during the randomized treatment period were due to disease under study.
The most common Grade 3/4 hematologic lab abnormality (>10% in either arm) during randomized treatment period was lymphocytes decrease (38.1% vs. 2.7%) on the Lutathera and the High-dose Octreotide arms, respectively. Less than 5% of subjects showed Grade 3/4 lab abnormalities for any of the other hematologic parameters.
The most common Grade 3/4 biochemical lab abnormalities (>5% in either arm) during randomized treatment period were GGT increase (25.9% vs. 28.8%), aspartate aminotransferase increase (2.7% vs. 6.8%), and bilirubin increase (2.7% vs. 5.5%) on the Lutathera and the High-dose Octreotide arms, respectively.

### Additional Information

Below are the treatment group labels used in the report:

| Study treatment group | Treatment group labels in the report |
|---|---|
| Lutathera + Octreotide LAR 30 mg | Lutathera |
| High-dose Octreotide LAR 60 mg | Octreotide LAR |
| All subjects | All subjects |

### Subjects Studied

### Subject Disposition

### Subject Disposition (Full Analysis Set)

| | | Octreotide | |
|---|---|---|---|
| | Lutathera | LAR | All subjects |
| | N = 151 | N = 75 | N = 226 |
| | n (%) | n (%) | n (%) |
| Subjects Treated in treatment period | 147 (97.4) | 73 (97.3) | 220 (97.3) |
| Subjects Currently in Initial Treatment period* | 78 (51.7) | 15 (20.0) | 93 (41.2) |
| Subjects Currently in Cross-over Treatment period* | NA | 10 (13.3) | 10 (4.4) |
| Subjects Currently in Re-treatment period* | 5 (3.3) | NA | 5 (2.2) |
| Subjects Currently in Follow-up period* | 32 (21.2) | 29 (38.7) | 61 (27.0) |
| Subjects Discontinued from Study | 36 (23.8) | 21 (28.0) | 57 (25.2) |
| Subjects Discontinued Randomized Treatment Phase | 73 (48.3) | 60 (80.0) | 133 (58.8) |
| Reason for End of Randomized Treatment Phase | | | |
| Progressive Disease | 42 (27.8) | 44 (58.7) | 86 (38.1) |
| Physician Decision | 13 (8.6) | 7 (9.3) | 20 (8.8) |
| Adverse Event | 8 (5.3) | 1 (1.3) | 9 (4.0) |
| Death | 4 (2.6) | 4 (5.3) | 8 (3.5) |
| Withdrawal by subject | 3 (2.0) | 4 (5.3) | 7 (3.1) |
| Other | 3 (2.0) | 0 | 3 (1.3) |

*Currently at the time of the data cut-off date 2023 Jul. 20

### Analysis Sets

### Analysis Sets (Full Analysis Set)

| | | Octreotide | All |
|---|---|---|---|
| | Lutathera | LAR | subjects |
| | N = 151 | N = 75 | N = 226 |
| Analysis set | n (%) | n (%) | n (%) |
| Full Analysis Set | 151 (100) | 75 (100) | 226 (100) |
| Safety Set | 147 (97.4) | 73 (97.3) | 220 (97.3) |
| Cross-over Set | NA | 29 (38.7) | 29 (12.8) |
| Re-treatment Set | 8 (5.3) | NA | 8 (3.5) |

The Full Analysis Set (FAS) comprises all subjects to whom study treatment has been assigned by randomization.
The Safety Set includes all subjects who received any study treatment
The Cross-over Set includes all subjects who received at least one dose of Lutathera after centrally evaluated progression per RECIST after receiving treatment in the Octreotide LAR arm
The Re-treatment Set includes all subjects who received at least one dose of Lutathera after centrally evaluated progression per RECIST after receiving treatment in the Lutathera arm

### Randomization Stratification Factors (Full Analysis Set)

| | | | | Octreotide | All |
|---|---|---|---|---|---|
| | | | Lutathera | LAR | subjects |
| | Tumor | Tumor | N = 151 | N = 75 | N = 226 |
| Strata | Grade | Origin | n (%) | n (%) | n (%) |
| 1 | G2 | pNET | 50 (33.1) | 26 (34.7) | 76 (33.6) |
| 2 | G2 | Other | 47 (31.1) | 22 (29.3) | 69 (30.5) |
| 3 | G3 | pNET | 32 (21.2) | 16 (21.3) | 48 (21.2) |
| 4 | G3 | Other | 22 (14.6) | 11 (14.7) | 33 (14.6) |
| Summary | G2 | — | 97 (64.2) | 48 (64.0) | 145 (64.2) |
| | G3 | — | 54 (35.8) | 27 (36.0) | 81 (35.8) |
| | — | pNET | 82 (54.3) | 42 (56.0) | 124 (54.9) |
| | — | Other | 69 (45.7) | 33 (44.0) | 102 (45.1) |

Strata as entered in the IRT during randomization

### Demographics and Other Baseline Characteristics

Demographics and baseline characteristics (Full analysis set)

| | | Octreotide | |
|---|---|---|---|
| | Lutathera | LAR | All subjects |
| Demographic variable | N = 151 | N = 75 | N = 226 |
| Age (years) | | | |
| N | 151 | 75 | 226 |
| Mean (SD) | 60.2 (13.21) | 59.6 (11.52) | 60.0 (12.65) |
| Median | 61.0 | 60.0 | 61.0 |
| Q1-Q3 | 51.0-72.0 | 51.0-69.0 | 51.0-70.0 |
| Min-Max | 23-88 | 34-82 | 23-88 |
| Age (categorized)-n (%) | | | |
| Age Group 2 | | | |
| 18-64 years | 86 (57.0) | 48 (64.0) | 134 (59.3) |
| >=65-74 years | 45 (29.8) | 19 (25.3) | 64 (28.3) |
| 75-84 years | 19 (12.6) | 8 (10.7) | 27 (11.9) |
| >=85 years | 1 (0.7) | 0 | 1 (0.4) |
| Sex-n (%) | | | |
| Male | 81 (53.6) | 40 (53.3) | 121 (53.5) |
| Female | 70 (46.4) | 35 (46.7) | 105 (46.5) |
| Race-n (%) | | | |
| White | 115 (76.2) | 50 (66.7) | 165 (73.0) |
| Black or African American | 3 (2.0) | 2 (2.7) | 5 (2.2) |
| Asian | 23 (15.2) | 11 (14.7) | 34 (15.0) |
| Indian | 2 (1.3) | 1 (1.3) | 3 (1.3) |
| Korean | 21 (13.9) | 10 (13.3) | 31 (13.7) |
| Native Hawaiian or other Pacific Islander | 1 (0.7) | 0 | 1 (0.4) |
| American Indian or Alaska Native | 1 (0.7) | 0 | 1 (0.4) |
| Missing | 8 (5.3) | 12 (16.0) | 20 (8.8) |
| Karnofsky performance score at baseline-n (%) | | | |
| 60 | 0 | 1 (1.3) | 1 (0.4) |
| 70-80 | 28 (18.5) | 10 (13.3) | 38 (16.8) |
| 90-100 | 123 (81.5) | 64 (85.3) | 187 (82.7) |

### Disease History

### Disease History (Full Analysis Set)

| | | Octreotide | |
|---|---|---|---|
| | Lutathera | LAR | All subjects |
| Disease history | N = 151 | N = 75 | N = 226 |
| Primary tumor site (GEP-NET)-n (%) | | | |
| Pancreas | 82 (54.3) | 41 (54.7) | 123 (54.4) |
| Small Intestine | 45 (29.8) | 21 (28.0) | 66 (29.2) |
| Other | 24 (15.9) | 13 (17.3) | 37 (16.4) |
| Presence of metastases-n (%) | | | |
| Yes | 150 (99.3) | 74 (98.7) | 224 (99.1) |
| No | 1 (0.7) | 1 (1.3) | 2 (0.9) |
| Site of Metastases-n (%) | | | |
| Bone | 37 (24.5) | 18 (24.0) | 55 (24.3) |
| Colon | 3 (2.0) | 0 | 3 (1.3) |
| Ileum | 5 (3.3) | 4 (5.3) | 9 (4.0) |
| Jejunum | 2 (1.3) | 1 (1.3) | 3 (1.3) |
| Kidney | 1 (0.7) | 0 | 1 (0.4) |
| Liver | 134 (88.7) | 69 (92.0) | 203 (89.8) |
| Lung | 15 (9.9) | 2 (2.7) | 17 (7.5) |
| Skin | 1 (0.7) | 0 | 1 (0.4) |
| Lymph node distant | 37 (24.5) | 14 (18.7) | 51 (22.6) |
| Lymph nodes regional | 88 (58.3) | 32 (42.7) | 120 (53.1) |
| Mediastinum | 1 (0.7) | 2 (2.7) | 3 (1.3) |
| Pelvis (non bone) | 4 (2.6) | 3 (4.0) | 7 (3.1) |
| Peritoneum | 26 (17.2) | 9 (12.0) | 35 (15.5) |
| Pleura | 0 | 1 (1.3) | 1 (0.4) |
| Spleen | 4 (2.6) | 0 | 4 (1.8) |
| Other | 25 (16.6) | 6 (8.0) | 31 (13.7) |
| Missing | 1 (0.7) | 1 (1.3) | 2 (0.9) |
| Histopathology grade at diagnosis per eCRF-n (%) | | | |
| G2 | 99 (65.6) | 48 (64.0) | 147 (65.0) |
| G3 | 52 (34.4) | 27 (36.0) | 79 (35.0) |
| Tumor Origin by Grade per eCRF-n (%) | | | |
| pNET G2 | 50 (33.1) | 26 (34.7) | 76 (33.6) |
| Small intestine G2 | 36 (23.8) | 12 (16.0) | 48 (21.2) |
| Other G2 | 13 (8.6) | 10 (13.3) | 23 (10.2) |
| pNET G3 | 32 (21.2) | 15 (20.0) | 47 (20.8) |
| Small intestine G3 | 9 (6.0) | 9 (12.0) | 18 (8.0) |
| Other G3 | 11 (7.3) | 3 (4.0) | 14 (6.2) |
| Time since initial diagnosis of GEP-NET to study entry (months) | | | |
| N | 151 | 74 | 225 |
| Mean (SD) | 5.1 (12.11) | 6.5 (16.91) | 5.6 (13.85) |
| Median | 1.8 | 2.1 | 1.9 |
| Q1-Q3 | 1.2-3.7 | 1.4-3.9 | 1.3-3.7 |
| Min-Max | 0-102 | 0-94 | 0-102 |
| Disease stage-n (%) | | | |
| 1 | 0 | 0 | 0 |
| IIA | 0 | 1 (1.3) | 1 (0.4) |
| IIB | 0 | 0 | 0 |
| IIIA | 1 (0.7) | 0 | 1 (0.4) |
| IIIB | 2 (1.3) | 0 | 2 (0.9) |
| IV | 148 (98.0) | 74 (98.7) | 222 (98.2) |
| Highest SSTR Tumor uptake score*-n (%) | | | |
| Grade 3 | 56 (37.1) | 25 (33.3) | 81 (35.8) |
| Grade 4 | 95 (62.9) | 50 (66.7) | 145 (64.2) |
| Extent of overall tumor burden per central assessment-n (%) | | | |
| Limited | 17 (11.3) | 9 (12.0) | 26 (11.5) |
| Moderate | 69 (45.7) | 36 (48.0) | 105 (46.5) |
| Extensive | 65 (43.0) | 29 (38.7) | 94 (41.6) |
| Missing | 0 | 1 (1.3) | 1 (0.4) |
| Results of Ki67 | | | |
| Mean (SD) | 19.7 (10.08) | 20.0 (10.42) | 19.8 (10.17) |
| Median | 17.0 | 16.0 | 16.0 |
| Q1-Q3 | 12.0-25.0 | 12.0-25.0 | 12.0-25.0 |
| Min-Max | 10-50 | 10-50 | 10-50 |

*Highest SSTR tumor update score is based on the local assessment.

### Subject Exposure

### Lutathera Exposure in the Treatment Arm (Safety Set)

| | Lutathera |
|---|---|
| | (N = 147) |
| Randomized treatment period | |
| Duration of exposure (weeks) | |
| N | 147 |
| Mean | 31.11 |
| SD | 4.971 |
| Median | 32.00 |
| Min-Max | 8.0-40.0 |
| Number of cycles started by subject, n (%) | |
| 1 cycle | 1 (0.7) |
| 2 cycles | 10 (6.8) |
| 3 cycles | 7 (4.8) |
| 4 cycles | 129 (87.8) |
| Cumulative Dose (GBq) | |
| Mean | 27.57 |
| SD | 4.594 |
| Median | 29.21 |
| Min-Max | 7.0-31.2 |
| Dose per administration (GBq/cycle) | |
| Mean | 7.25 |
| SD | 0.387 |
| Median | 7.34 |
| Min-Max | 4.1-7.8 |

Lutathera cycles are once every 8 weeks for a maximum of 4 cycles.

### Octreotide Exposure, in the Treatment Period (Safety Set)

| | | Octreotide |
|---|---|---|
| | Lutathera | LAR |
| | (N = 147) | (N = 73) |
| Randomized treatment period | | |
| Duration of exposure (weeks) | | |
| N | 147 | 73 |
| Mean | 75.89 | 46.90 |
| SD | 37.135 | 30.181 |
| Median | 71.00 | 40.29 |
| Min-Max | 8.0-182.7 | 4.0-124.4 |

Octreotide cycles are every 8 weeks for the 1^{st} 4 cycles, and then are every 4 weeks in the Lutathera arm.
In the Octreotide LAR arm, Octreotide cycles are every 4 weeks.

### Efficacy Results

### Primary Efficacy Results

Kaplan-Meier estimates of Progression-Free Survival (PFS) (months) based on central assessment using RECIST 1.1 criteria (Full Analysis Set)

| | | Octreotide |
|---|---|---|
| | Lutathera | LAR |
| Estimates | N = 151 | N = 75 |
| Progression-free Survival (PFS), n (%) | | |
| Events | 55 (36.4) | 46 (61.3) |
| Progression | 47 (31.1) | 41 (54.7) |
| Deaths | 8 (5.3) | 5 (6.7) |
| Censored | 96 (63.6) | 29 (38.7) |
| Ongoing without event | 70 (46.4) | 14 (18.7) |
| Lost to follow-up | 0 | 0 |
| Adequate assessment no longer available | 2 (1.3) | 1 (1.3) |
| Withdrew consent | 3 (2.0) | 2 (2.7) |
| New Anti-Cancer Therapy | 21 (13.9) | 12 (16.0) |
| Event after Two or More Missed Visits (1) | 0 | 0 |
| Percentiles for PFS (95%) (months) (2) | | |
| 25th percentile | 14.1 (10.9, 17.0) | 3.9 (3.7, 5.7) |
| Median PFS | 22.8 (19.4, NE) | 8.5 (7.7, 13.8) |
| 75th percentile | NE (29.0, NE) | 19.3 (13.8, NE) |
| Kaplan-Meier event-free estimates (95% CI) (3) | | |
| 4 months | 95.8 (91.0, 98.1) | 73.5 (61.2, 82.4) |
| 6 months | 92.3 (86.6, 95.7) | 64.5 (51.8, 74.6) |
| 9 months | 85.6 (78.6, 90.5) | 46.0 (33.4, 57.7) |
| 12 months | 80.7 (72.9, 86.4) | 42.4 (30.0, 54.3) |
| 15 months | 71.0 (61.9, 78.3) | 36.4 (24.3, 48.5) |
| 18 months | 66.5 (56.9, 74.4) | 29.2 (17.7, 41.7) |
| 24 months | 49.8 (38.5, 60.1) | 19.5 (9.1, 32.7) |
| 30 months | 36.1 (22.3, 50.1) | NE (NE, NE) |
| 36 months | NE (NE, NE) | NE (NE, NE) |
| Hazard ratio (Stratified Cox PH model) (4, 5) | | |
| 95% CI | 0.276 (0.182, 0.418) | |
| Hazard ratio (Unstratified Cox PH model) (4) | | |
| 95% CI | 0.309 (0.207, 0.462) | |
| Log-rank test (one-sided) | | |
| Stratified p-value (5) | <0.0001 | |
| Unstratified P-value | <0.0001 | |
| Follow-up time (months) (6) | | |
| Mean (SD) | 15.2 (8.47) | 9.0 (6.94) |
| Median | 14.1 | 8.0 |
| Min-Max | 0.0-36.0 | 0.0-28.0 |

(1) Refer to other paragraphs of this disclosure of the SAP for details of calculation.
(2) Percentiles with 95% CIs are calculated from PROC LIFETEST output using method of Brookmeyer and Crowley (1982).
(3) % Kaplan-Meier Event-free estimate is the estimated probability that a subject will remain event-free up to the specified time point. Greenwood formula is used for CIs of KM event-free estimates.
(4) Hazard Ratio of Lutathera versus Octreotide LAR.
(5) Both Log-rank test and Cox PH model are stratified based on the randomization stratification factors: tumor grade (G2 vs G3) and tumor origin (pNET vs Other origin) per IRT.
(6) Follow-up time = (Date of event or censoring − randomization date + 1)/30.4375 (months)

Kaplan-Meier plot of Progression-Free Survival (PFS) (months) based on central review and using RECIST 1.1 criteria (Full Analysis Set): see FIG. 2.

Forest plot of Progression-Free Survival (PFS) (months) based on central review and using RECIST 1.1 criteria-Key subgroups of interest (Full Analysis Set): see FIGS. 3 and 4.

### Key Secondary Efficacy Results

### Objective Response Rate

Best overall response based on central review using RECIST 1.1 criteria (Full Analysis Set)

| | | Octreotide |
|---|---|---|
| | Lutathera | LAR |
| | N = 151 | N = 75 |
| Best Overall Response (BOR), n (%) | | |
| Complete Response (CR) | 8 (5.3) | 0 |
| Partial Response (PR) | 57 (37.7) | 7 (9.3) |
| Stable Disease (SD) | 72 (47.7) | 42 (56.0) |
| Non-CR/Non-PD | 0 | 1 (1.3) |
| Progressive Disease (PD) | 8 (5.3) | 14 (18.7) |
| Unknown | 6 (4.0) | 11 (14.7) |
| Overall Response Rate (ORR: CR + PR), n (%) [95%] | 65 (43.0) | 7 (9.3) |
| | (35.0, 51.3) | (3.8, 18.3) |
| Stratified Odds Ratio [95% CI] (1) | 7.81 (3.32, 18.40) | |
| Stratified One-sided p-value (1) | <0.0001 | |
| Unstratified Odds Ratio [95% CI] | 7.34 (3.16, 17.04) | |
| Unstratified One-sided p-value | <0.0001 | |
| Disease Control Rate (DCR: | 137 (90.7) | 50 (66.7) |
| CR+PR+SD+Non-CR/Non-PD), n (%) [95% CI] | (84.9, 94.8) | (54.8, 77.1) |

(1) Odds Ratio of Lutathera vs. Octreotide LAR based on stratified CMH method using IRT stratification factors: tumor grade (G2 vs G3) and tumor origin (pNET vs Other origin) per IRT. P-value based on CMH Chi-Square test.

### Safety Results

### Adverse Events

### Overview of Adverse Events in the Randomized Treatment Period (Safety Set)

| | | | Octreotide | |
|---|---|---|---|---|
| | Lutathera | | LAR | |
| | N = 147 | | N = 73 | |
| | All grades | Grade >=3 | All grades | Grade >=3 |
| Category | n (%) | n (%) | n (%) | n (%) |
| Adverse events | 136 (92.5) | 52 (35.4) | 69 (94.5) | 20 (27.4) |
| Related to Any Treatment | 101 (68.7) | 23 (15.6) | 43 (58.9) | 3 (4.1) |
| Related to Lutathera | 96 (65.3) | 22 (15.0) | | |
| Related to Octreotide | 55 (37.4) | 2 (1.4) | 43 (58.9) | 3 (4.1) |
| Related to Amino Acids | 22 (15.0) | 1 (0.7) | | |
| SAEs | 30 (20.4) | 24 (16.3) | 15 (20.5) | 13 (17.8) |
| Related to Any Treatment | 8 (5.4) | 6 (4.1) | 1 (1.4) | 1 (1.4) |
| Related to Lutathera | 8 (5.4) | 6 (4.1) | | |
| Related to Octreotide | 0 | 0 | 1 (1.4) | 1 (1.4) |
| Related to Amino Acids | 0 | 0 | | |
| Fatal SAEs | 3 (2.0) | 3 (2.0) | 2 (2.7) | 2 (2.7) |
| Related to Any Treatment | 0 | 0 | 0 | 0 |
| Related to Lutathera | 0 | 0 | | |
| Related to Octreotide | 0 | 0 | 0 | 0 |
| Related to Amino Acids | 0 | 0 | | |
| AEs leading to discontinuation of | | | | |
| Any Treatment (Lutathera, Octreotide) | 7 (4.8) | 4 (2.7) | 2 (2.7) | 2 (2.7) |
| Lutathera | 3 (2.0) | 1 (0.7) | | |
| Octreotide | 5 (3.4) | 3 (2.0) | 2 (2.7) | 2 (2.7) |
| AEs leading to dose reduction of | | | | |
| Any Treatment (Lutathera, Octreotide) | 3 (2.0) | 1 (0.7) | 1 (1.4) | 1 (1.4) |
| Lutathera | 2 (1.4) | 1 (0.7) | | |
| Octreotide | 1 (0.7) | 0 | 1 (1.4) | 1 (1.4) |
| AEs leading to dose interruption of | | | | |
| Any Treatment (Lutathera, Octreotide) | 23 (15.6) | 7 (4.8) | 11 (15.1) | 5 (6.8) |
| Lutathera | 14 (9.5) | 3 (2.0) | | |
| Octreotide | 19 (12.9) | 7 (4.8) | 11 (15.1) | 5 (6.8) |
| AEs requiring additional therapy* | 109 (74.1) | 28 (19.0) | 56 (76.7) | 17 (23.3) |

Randomized treatment period starts from first dose of study treatment to last dose of study treatment in the randomized treatment phase + 30 days before the 1st injection of Lutathera in extension (cross-over/re-treatment) phase.
*Additional therapy includes concomitant medication, surgery, non-drug therapy or Other as collected in AE eCRF page.

### Adverse Events by Preferred Term in the Randomized Treatment Period (at Least 10% Incidence in Either Arm) (Safety Set)

| | | | Octreotide | |
|---|---|---|---|---|
| | Lutathera | | LAR | |
| | N = 147 | | N = 73 | |
| | All grades | Grade >=3 | All grades | Grade >=3 |
| Preferred term | n (%) | n (%) | n (%) | n (%) |
| Number of subjects | 136 (92.5) | 52 (35.4) | 69 (94.5) | 20 (27.4) |
| with at least one event | | | | |
| Nausea | 40 (27.2) | 1 (0.7) | 13 (17.8) | 0 |
| Diarrhoea | 38 (25.9) | 2 (1.4) | 25 (34.2) | 1 (1.4) |
| Anaemia | 29 (19.7) | 1 (0.7) | 5 (6.8) | 1 (1.4) |
| Fatigue | 29 (19.7) | 0 | 13 (17.8) | 0 |
| Asthenia | 28 (19.0) | 1 (0.7) | 9 (12.3) | 0 |
| COVID-19 | 28 (19.0) | 0 | 10 (13.7) | 0 |
| Abdominal pain | 26 (17.7) | 4 (2.7) | 20 (27.4) | 3 (4.1) |
| Platelet count decreased | 25 (17.0) | 2 (1.4) | 4 (5.5) | 0 |
| Alopecia | 22 (15.0) | 0 | 1 (1.4) | 0 |
| Vomiting | 21 (14.3) | 1 (0.7) | 6 (8.2) | 1 (1.4) |
| Hyperglycaemia | 20 (13.6) | 0 | 11 (15.1) | 2 (2.7) |
| Decreased appetite | 19 (12.9) | 0 | 7 (9.6) | 0 |
| White blood cell count decreased | 19 (12.9) | 3 (2.0) | 3 (4.1) | 0 |
| Weight increased | 18 (12.2) | 2 (1.4) | 5 (6.8) | 0 |
| Oedema peripheral | 17 (11.6) | 0 | 6 (8.2) | 1 (1.4) |
| Alanine aminotransferase increased | 16 (10.9) | 1 (0.7) | 10 (13.7) | 2 (2.7) |
| Aspartate aminotransferase increased | 16 (10.9) | 1 (0.7) | 8 (11.0) | 2 (2.7) |
| Headache | 16 (10.9) | 0 | 5 (6.8) | 0 |
| Lymphocyte count decreased | 16 (10.9) | 8 (5.4) | 0 | 0 |
| Constipation | 15 (10.2) | 1 (0.7) | 6 (8.2) | 0 |
| Gamma-glutamyltransferase increased | 12 (8.2) | 7 (4.8) | 8 (11.0) | 2 (2.7) |

Randomized treatment period starts from first dose of study treatment to last dose of study treatment in the randomized treatment phase + 30 days before the 1st injection of Lutathera in extension (cross-over/re-treatment) phase.
Preferred terms are presented by descending frequency in the all grade column in Lutathera arm.
Numbers (n) represent counts of subjects.
MedDRA version 26.0

### Serious Adverse Events by Preferred Term in the Randomized Treatment Period (>1% Incidence in Either Arm) (Safety Set)

| | | | Octreotide | |
|---|---|---|---|---|
| | Lutathera | | LAR | |
| | N = 147 | | N = 73 | |
| | All grades | Grade >=3 | All grades | Grade >=3 |
| Preferred term | n (%) | n (%) | n (%) | n (%) |
| Number of subjects | 30 (20.4) | 24 (16.3) | 15 (20.5) | 13 (17.8) |
| with at least one event | | | | |
| Small intestinal obstruction | 5 (3.4) | 5 (3.4) | 0 | 0 |
| Abdominal pain | 2 (1.4) | 2 (1.4) | 0 | 0 |
| Anaemia | 2 (1.4) | 1 (0.7) | 1 (1.4) | 1 (1.4) |
| Blood bilirubin increased | 2 (1.4) | 1 (0.7) | 1 (1.4) | 1 (1.4) |
| Gastrointestinal stoma complication | 2 (1.4) | 2 (1.4) | 0 | 0 |
| Pyrexia | 2 (1.4) | 1 (0.7) | 0 | 0 |
| Ascites | 1 (0.7) | 0 | 1 (1.4) | 1 (1.4) |
| Diarrhoea | 1 (0.7) | 0 | 1 (1.4) | 1 (1.4) |
| Intestinal obstruction | 1 (0.7) | 0 | 1 (1.4) | 1 (1.4) |
| Oedema peripheral | 1 (0.7) | 0 | 1 (1.4) | 1 (1.4) |
| Abdominal sepsis | 0 | 0 | 1 (1.4) | 1 (1.4) |
| Acute coronary syndrome | 0 | 0 | 1 (1.4) | 0 |
| Acute kidney injury | 0 | 0 | 1 (1.4) | 1 (1.4) |
| Carcinoid heart disease | 0 | 0 | 1 (1.4) | 1 (1.4) |
| Carcinoid syndrome | 0 | 0 | 1 (1.4) | 1 (1.4) |
| Femur fracture | 0 | 0 | 1 (1.4) | 0 |
| Gastroenteritis | 0 | 0 | 1 (1.4) | 1 (1.4) |
| Generalised oedema | 0 | 0 | 1 (1.4) | 1 (1.4) |
| Hip fracture | 0 | 0 | 1 (1.4) | 1 (1.4) |
| Hyperglycaemia | 0 | 0 | 1 (1.4) | 1 (1.4) |
| Hypertension | 0 | 0 | 1 (1.4) | 1 (1.4) |
| Hypoalbuminaemia | 0 | 0 | 1 (1.4) | 1 (1.4) |
| Jaundice | 0 | 0 | 1 (1.4) | 1 (1.4) |
| Lower gastrointestinal haemorrhage | 0 | 0 | 1 (1.4) | 1 (1.4) |
| Metabolic encephalopathy | 0 | 0 | 1 (1.4) | 1 (1.4) |
| Pleurisy | 0 | 0 | 1 (1.4) | 1 (1.4) |
| Tumour lysis syndrome | 0 | 0 | 1 (1.4) | 1 (1.4) |

Randomized treatment period starts from first dose of study treatment to last dose of study treatment in the randomized treatment phase + 30 days before the 1st injection of Lutathera in extension (cross-over/re-treatment) phase.
Preferred terms are presented by descending frequency in the all grade column in Lutathera arm.
Numbers (n) represent counts of subjects.
MedDRA version 26.0

### Deaths

### All Deaths (Safety Set)

| | | Octreotide |
|---|---|---|
| | Lutathera | LAR |
| Category | N = 147 | N = 73 |
| Primary reason | n (%) | n (%) |
| Number of Subjects who Died (Overall) | 32 (21.8) | 14 (19.2) |
| Disease under Study | 30 (20.4) | 13 (17.8) |
| Adverse Event | 2 (1.4) | 1 (1.4) |
| Number of Subjects who Died on-treatment in randomized treatment | | |
| period (within 30 days of last dose) | | |
| Disease under Study | 2 (1.4) | 4 (5.5) |
| Number of Subjects who Died on-treatment in cross-over period | | |
| (within 30 days of last dose) | | |
| Disease under Study | NA | 1 (1.4) |
| Number of Subjects who Died on-treatment in re-treatment period | 0 | NA |
| (within 30 days of last dose) | | |
| Number of subjects who died in the post-treatment follow up period | 30 (20.4) | 9 (12.3) |
| Disease under Study | 28 (19.0) | 8 (11.0) |
| Adverse Event | 2 (1.4) | 1 (1.4) |

Randomized treatment period starts from first dose of study treatment to last dose of study treatment in the randomized treatment phase + 30 days before the 1st injection of Lutathera in extension (cross-over/re-treatment) phase.
Cross-over period starts from the first administration of Lutathera in Cross-over, to the date of last administration of study treatment + 30 days
Re-treatment period starts from the first administration of Lutathera in Re-treatment, to the date of last administration of study treatment + 30 days.
Post-treatment follow up period starts from last study treatment date in randomized, re-treatment or cross-over phase (whichever occurs later) + 30 days.
Numbers (n) represent counts of subjects.
MedDRA version 26.0

Serious Adverse Events with Fatal Outcome in the Randomized Treatment Period (Safety Set)

| | | Octreotide |
|---|---|---|
| | Lutathera | LAR |
| Category | N = 147 | N = 73 |
| Preferred term | n (%) | n (%) |
| SAEs with fatal outcome | 3 (2.0) | 2 (2.7) |
| Reported in subjects with primary | 3 (2.0) | 2 (2.7) |
| reason for death = Disease under Study | | |
| Blood bilirubin increased | 1 (0.7) | 0 |
| Dyspnoea | 1 (0.7) | 0 |
| Intestinal perforation | 1 (0.7) | 0 |
| Diarrhoea | 0 | 1 (1.4) |
| Tumor Lysis syndrome | 0 | 1 (1.4) |

Randomized treatment period starts from first dose of study treatment to last dose of study treatment in the randomized treatment phase + 30 days before the 1st injection of Lutathera in extension (cross-over/re-treatment) phase.
Numbers (n) represent counts of subjects.
A subject may have more than one SAE with fatal outcome.
MedDRA version 26.0

### Laboratory Data

### Worst Post-Baseline Biochemistry Abnormalities Based on CTC Grades in the Randomized Treatment Period (Safety Set)

| | | | Octreotide | |
|---|---|---|---|---|
| | Lutathera | | LAR | |
| | N = 147 | | N = 73 | |
| | All grades | Grade 3/4 | All grades | Grade 3/4 |
| | n (%) | n (%) | n (%) | n (%) |
| Creatinine (umol/L)-Increase | 30 (20.4) | 0 | 15 (20.5) | 1 (1.4) |
| Albumin (g/L)-Decrease | 25 (17.0) | 0 | 17 (23.3) | 1 (1.4) |
| Bilirubin (umol/L)-Increase | 26 (17.7) | 4 (2.7) | 17 (23.3) | 4 (5.5) |
| Alkaline Phosphatase (U/L)-Increase | 79 (53.7) | 7 (4.8) | 40 (54.8) | 3 (4.1) |
| Aspartate Aminotransferase (U/L)-Increase | 75 (51.0) | 4 (2.7) | 40 (54.8) | 5 (6.8) |
| Alanine Aminotransferase (U/L)-Increase | 63 (42.9) | 6 (4.1) | 38 (52.1) | 3 (4.1) |
| Gamma Glutamyl Transferase (U/L)-Increase | 102 (69.4) | 38 (25.9) | 56 (76.7) | 21 (28.8) |
| Sodium (mmol/L)-Increase | 5 (3.4) | 0 | 6 (8.2) | 0 |
| Sodium (mmol/L)-Decrease | 32 (21.8) | 1 (0.7) | 12 (16.4) | 1 (1.4) |
| Potassium (mmol/L)-Increase | 33 (22.4) | 3 (2.0) | 16 (21.9) | 3 (4.1) |
| Potassium (mmol/L)-Decrease | 13 (8.8) | 4 (2.7) | 10 (13.7) | 2 (2.7) |
| Calcium Corrected (mmol/L)-Increase | 9 (6.1) | 0 | 8 (11.0) | 1 (1.4) |

Randomized treatment period starts from first dose of study treatment to last dose of study treatment in the randomized treatment phase + 30 days before the 1st injection of Lutathera in extension (cross-over/re-treatment) phase.
Numbers (n) represent counts of subjects. 'All grades' represents subjects with any grade 1, 2, 3 or 4 post-baseline.
Grades based on CTCAE version 5.0.

### Worst Post-Baseline Hematology Abnormalities Based on CTC Grades in the Randomized Treatment Period (Safety Set)

| | | | Octreotide | |
|---|---|---|---|---|
| | Lutathera | | LAR | |
| | N = 147 | | N = 73 | |
| | All grades | Grade 3/4 | All grades | Grade 3/4 |
| | n (%) | n (%) | n (%) | n (%) |
| Leukocytes (10E9/L)-Increase | 0 | 0 | 0 | 0 |
| Leukocytes (10E9/L)-Decrease | 92 (62.6) | 6 (4.1) | 15 (20.5) | 0 |
| Lymphocytes (10E9/L)-Increase | 4 (2.7) | 0 | 3 (4.1) | 0 |
| Lymphocytes (10E9/L)-Decrease | 142 (96.6) | 56 (38.1) | 45 (61.6) | 2 (2.7) |
| Neutrophils (10E9/L)-Decrease | 55 (37.4) | 5 (3.4) | 9 (12.3) | 0 |
| Platelets (10E9/L)-Decrease | 81 (55.1) | 3 (2.0) | 15 (20.5) | 0 |
| Hemoglobin (g/L)-Increase | 4 (2.7) | 0 | 2 (2.7) | 0 |
| Hemoglobin (g/L)-Decrease | 116 (78.9) | 2 (1.4) | 42 (57.5) | 2 (2.7) |

Randomized treatment period starts from first dose of study treatment to last dose of study treatment in the randomized treatment phase + 30 days before the 1st injection of Lutathera in extension (cross-over/re-treatment) phase.
Numbers (n) represent counts of subjects.
‘All grades’ represents subjects with any grade 1, 2, 3 or 4 post-baseline.
Grades based on CTCAE version 5.0.

1-128. (canceled)

129. A method of treating well-differentiated G2 or G3 neuroendocrine tumor (NET) in a patient in need thereof, comprising administering to said patient a treatment comprising a therapeutically effective dose of lutetium (177Lu) oxodotreotide, as first line therapy.

130. The method of claim 129, wherein the dose of the lutetium (177Lu) oxodotreotide is from 5 to 10 GBq.

131. The method of claim 129, wherein said dose is administered once every 40 to 100 days.

132. The method of claim 129, wherein the lutetium (177Lu) oxodotreotide is administered at a dose of 7.4±10% GBq every 8±1 weeks for up to 4 times (4 cycles).

133. The method of claim 129, wherein the treatment is characterized by a disease control rate (DCR, i.e. complete response (CR) rate+partial response (PR) rate)+stable disease (SD) rate) of at least 50%.

134. The method of claim 129, wherein the treatment is characterized by an objective response rate (ORR, i.e. complete response (CR) rate+partial response (PR) rate) of at least 25%.

135. The method of claim 129, wherein the treatment reduces the risk of progression or death by at least 50% compared to a comparable treatment without the lutetium (177Lu) oxodotreotide.

136. The method of claim 129, wherein the treatment reduces the risk of progression or death corresponding to a hazard ratio (HR) of less than 0.5, (95% Cl) when the risk is calculated relative to patients a comparable treatment without the lutetium (177Lu) oxodotreotide.

137. The method of claim 129, wherein the NET is a gastroentero-pancreatic NET (GEP-NET).

138. The method of claim 132, wherein the treatment further comprises administering 30 mg long-acting octreotide once every 8 weeks during the treatment with the lutetium (177Lu) oxodotreotide, and optionally once every 4 weeks after the 4 cycles of treatment of the lutetium (177Lu) oxodotreotide is completed.

139. A method of delaying the time-to-first-occurrence progression or death in a patient with well-differentiated G2 or G3 neuroendocrine tumor (NET), comprising administering to said patient a treatment comprising a therapeutically effective dose of lutetium (177Lu) oxodotreotide, as first line therapy.

140. The method of claim 139, wherein the dose of the lutetium (177Lu) oxodotreotide is from 5 to 10 GBq.

141. The method of claim 139, wherein said dose is administered once every 40 to 100 days.

142. The method of claim 139, wherein the lutetium (177Lu) oxodotreotide is administered at a dose of 7.4±10% GBq every 8±1 weeks for up to 4 times (4 cycles).

143. The method of claim 139, wherein the treatment is characterized by a disease control rate (DCR, i.e. complete response (CR) rate+partial response (PR) rate)+stable disease (SD) rate) of at least 50, %.

144. The method of claim 139, wherein the treatment is characterized by an objective response rate (ORR, i.e. complete response (CR) rate+partial response (PR) rate) of at least 25%.

145. The method of claim 139, wherein the treatment reduces the risk of progression or death by at least 50% compared to a comparable treatment without the lutetium (177Lu) oxodotreotide.

146. The method of claim 139, wherein the treatment reduces the risk of progression or death corresponding to a hazard ratio (HR) of less than 0.5 (95% Cl) when the risk is calculated relative to patients a comparable treatment without the lutetium (177Lu) oxodotreotide.

147. The method of claim 139, wherein the NET is a gastroentero-pancreatic NET (GEP-NET).

148. The method of claim 142, wherein the treatment further comprises administering 30 mg long-acting octreotide once every 8 weeks during the treatment with the lutetium (177Lu) oxodotreotide, and optionally once every 4 weeks after the 4 cycles of treatment of the lutetium (177Lu) oxodotreotide is completed.

149. A method of reducing the risk of progression of well-differentiated G2 or G3 neuroendocrine tumor (NET) in a patient in need thereof or reducing the risk of death of said patient, comprising administering to said patient a treatment lutetium (177Lu) oxodotreotide, as first line therapy.

150. The method of claim 149, wherein the dose of the lutetium (177Lu) oxodotreotide is from 5 to 10 GBq.

151. The method of claim 149, wherein said dose is administered once every 40 to 100 days.

152. The method of claim 149, wherein lutetium (177Lu) oxodotreotide is administered at a dose of 7.4±10% GBq every 8±1 weeks for up to 4 times (4 cycles).

153. The method of claim 149, wherein the treatment is characterized by a disease control rate (DCR, i.e. complete response (CR) rate+partial response (PR) rate)+stable disease (SD) rate) of at least 50%.

154. The method of claim 149, wherein the treatment is characterized by an objective response rate (ORR, i.e. complete response (CR) rate+partial response (PR) rate) of at least 25%.

155. The method of claim 149, wherein the treatment reduces the risk of progression or death by at least 50% compared to a comparable treatment without the lutetium (177Lu) oxodotreotide.

156. The method of claim 149, wherein the treatment reduces the risk of progression or death corresponding to a hazard ratio (HR) of less than 0.5 (95% Cl) when the risk is calculated relative to patients a comparable treatment without the lutetium (177Lu) oxodotreotide.

157. The method of claim 149, wherein the NET is a gastroentero-pancreatic NET (GEP-NET).

158. The method of claim 152, wherein the treatment further comprises administering 30 mg long-acting octreotide once every 8 weeks during the treatment with lutetium (177Lu) oxodotreotide, and optionally once every 4 weeks after the 4 cycles of treatment of the lutetium (177Lu) oxodotreotide is completed.
